(19) United States
(12) Patent Application Publication
(10) Pub. No.: US 2024/0197931 A1
(43) Pub. Date: Jun. 20, 2024

(54) RADIOIMMUNOTHERAPY DIRECTED TO CCR8 FOR DEPLETION OF TUMOR INFILTRATING REGULATORY T CELLS

(71) Applicant: ACTINIUM PHARMACEUTICALS, INC., New York, NY (US)

(72) Inventors: Dale L LUDWIG, Rockaway, NJ (US); Eileen GEOGHEGAN, New Rochelle, NY (US); Sandesh SETH, New York, NY (US); Jesse HWANG, Forest Hills, NY (US)

(73) Assignee: ACTINIUM PHARMACEUTICALS, INC., New York, NY (US)

(21) Appl. No.: 18/553,757

(22) PCT Filed: Apr. 7, 2022
(86) PCT No.: PCT/US2022/023885

§ 371 Date: Oct. 3, 2023

Related U.S. Application Data
(60) Provisional application No. 63/171,790, filed on Apr. 7, 2021.

(51) Int. Cl. A61K 51/10; A61K 45/06; C07K 16/28

(52) U.S. Cl. CPC A61K 51/1033; A61K 45/06; C07K 16/2803; C07K 16/2866; A61K 2121/00

(57) ABSTRACT

Provided are methods for treating a solid cancer in a subject by administering an effective amount of a radioconjugated CCR8-targeting agent to deplete tumor-associated CCR8-positive Treg cells, alone or in combination with one or more additional therapeutic agents or modalities, such as a radioconjugated CD33-targeting agent.

### CROSS-REFERENCE TO RELATED APPLICATIONS

This application claims priority to U.S. provisional application Ser. No. 63/171,790 filed Apr. 7, 2021, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Apr. 6, 2022, is named ATNM-019PCT_SL_ST25.txt and is 278,138 bytes in size.

### FIELD OF THE INVENTION

The present disclosure relates to the field of radiotherapeutics.

### BACKGROUND OF THE INVENTION

Chemokine (C-C Motif) Receptor 8 (CCR8) is a G-protein coupled receptor expressed predominantly on the surface of tumor infiltrating CD4+Foxp3+ regulatory T cells (Tregs; Lämmermann, 2019; Ohue, 2019; Magnuson, 2018; Zheng, 2017). Under non-malignant conditions, Tregs are crucial components of the immune system that prevent deleterious autoimmunity and help resolve inflammation (Vignali, 2008). However, Tregs can also inhibit antitumor response by promoting an immunosuppressive environment (Fridman, 2012; Tanaka, 2019; Nishikawa, 2010), and clinically, tumor infiltration of Tregs is associated with worse prognosis in patients with various cancers (Tanaka, 2018). Effective anti-tumor therapies would therefore need to specifically neutralize Treg cells that are present within the tumor microenvironment (TME) without affecting the homeostatic function of Tregs in healthy tissue.

Tregs, which are characterized by high expression of the transcription factor Foxp3, exert pro-tumorigenic effects through multiple mechanisms. For instance, Tregs are capable of killing NK cells and CD8 T cells through granzyme B and perforin, leading to compromised ability to clear tumors (Cao, 2007). In addition, the release of immunosuppressive cytokines (e.g., IL-10, IL-35, and TGF-β) maintains a microenvironment that favors tumor growth (Saleh, 2020). Moreover, Tregs compete with effector T cells for the mitogenic factor IL-2, leading to growth deprivation of effector T cells in favor of Treg survival (Saleh, 2020). Tregs also increase extracellular adenosine levels to cause immunosuppression, and express immune checkpoints (e.g., PD-1 and CTLA-4) to downregulate the activity of antigen presenting cells that are necessary for activation of effector T cells (Saleh, 2020). These immunosuppressive functions of Tregs may also curtail the clinical efficacy of adoptive cell transfer therapies, such as CAR T cell or NK cell therapies (Wang, 2019; Bagley, 2018; Epperly, 2020; Ogbomo, 2011). In brief, Treg-mediated mechanisms to prevent pathological inflammation in normal tissues are hijacked by tumors for uncontrolled growth.

Given the multitude of ways Tregs facilitate tumor proliferation, therapeutic removal of Tregs warranted further exploration. For instance, Treg depletion using CD25- or CTLA-4-targeting methods led to inhibition of tumor growth in preclinical models (Shimizu, 1999; Onizuka, 1999; Marabelle, 2013; Simpson, 2013). However, in addition to Tregs, effector T cells may also be removed in the process because CD25 and CTLA-4 can also be expressed on these cells (Couper, 2009; Peggs, 2009; De Simone, 2016), potentially leading to cancellation of any therapeutic benefit. Furthermore, there may be adverse events (e.g., autoimmune responses) that result from such a non-selective approach. To minimize the risk of adverse events and allow effector T cell populations to remain unperturbed, it is necessary to target Tregs that are present only within the TME by identifying a protein marker specifically expressed on these cells.

Recently, CCR8 has been identified to be highly and predominantly expressed in tumor-infiltrating Treg cells (De Simone, 2016; Plitas, 2016; Rankin, 2020). In a seminal study, De Simone et al. (2016) showed that tumor-infiltrating Tregs (obtained from multiple solid tumor samples) exhibit a unique gene signature compared to Tregs present in normal tissue. Of the differentially expressed genes, CCR8 was one of the genes with the most selective expression in tumor infiltrating Tregs. Interestingly, another group independently published similar findings at the same time. Focusing primarily on breast cancer (but validating in other solid tumor types), Plitas et al. (2016) showed that tumor infiltrating Tregs, but not activated peripheral blood Tregs, highly express CCR8. In addition, CCR8 was the most differentially expressed chemokine receptor in breast tumor infiltrating Tregs, similar to the findings by De Simone et al. (2016).

Furthermore, it was determined that activation of Tregs in the presence of tumor explants upregulates CCR8 to a greater extent compared to activation in the presence of normal tissue, indicating that the tumor microenvironment may directly contribute to CCR8 induction on Tregs (Plitas, 2016). The functional relevance of CCR8 induction in tumor infiltrating Tregs was demonstrated afterwards. In a study by Barsheshet et al. (2017), the investigators revealed that CCR8 forms a feed-forward regulatory axis with CCL1, an endogenous ligand of CCR8, to inhibit immune response in an autoimmune disease model. This study illuminates earlier findings where anti-CCL1 treatment led to tumor rejection in murine model by inhibiting Treg function without influencing effector T cells (Hoelzinger, 2010). Lastly, CCR8 expression is correlated with poor survival in multiple solid tumor types (e.g., NSCLC, colorectal cancer, breast cancer), demonstrating that CCR8 may be an attractive target for therapeutic intervention that would lead to beneficial clinical outcomes (De Simone, 2016; Plitas, 2016).

What is needed and provided by the present invention are new methods and compositions for treating cancers using radiolabeled CCR8 targeting agents.

### SUMMARY OF THE INVENTION

The present disclosure provides methods for treating and/or diagnosing cancers in mammalian subjects that involve the administration of one or more radiolabeled CCR8 targeting agents. The radioconjugated/radiolabeled CCR8 targeting agent may, for example, include a radiolabeled antibody, peptide or small molecule that binds to CCR8 on CCR8-expressing cells to deliver DNA-damage inducing radiation to CCR8-expressing cells, such as to tumor-infiltrating CCR8-positive Treg cells.

The present disclosure further provides methods of treating cancers such as solid tumors by targeting CCR8 with a radioconjugated CCR8 targeting agent. Such a method may target tumor-infiltrating CCR8-positive Treg cells and thus deplete such cells in the subject. The methods may further deplete bystander tumor cells, thus effecting overall tumor reduction. The radioconjugated CCR8-targeting agent may also increase the amount or activity of immune cells that produce antitumor immunity, such as, CD4 and/or CD8 T-cells.

A radioconjugated CCR8 targeting agent useful for diagnostics purposes may, for example, be an anti-CCR8 antibody, peptide, or small molecule including a radioisotope, such as ¹¹¹In, ⁶⁸Ga, ⁶⁴Cu ⁸⁹Zr, ¹²⁵I or ¹²³I.

A radioconjugated CCR8 targeting agent useful for cancer therapy may, for example, be an anti-CCR8 antibody, peptide, or small molecule including a radioisotope/radionuclide, such as: ¹³¹I, ¹²⁵I, ¹²⁴I, ⁹⁰Y, ¹⁷⁷Lu, ¹⁸⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb, or combinations thereof. The CCR8 targeting agent may, for example, be an antibody including ¹³¹I, ⁹⁰Y, ¹⁷⁷Lu, ²²⁵Ac, ²¹³Bi, ²¹¹At, ²²⁷Th, or ²¹²Pb as a radiolabel.

Therapeutic methods of the present disclosure may, for example, include administering to a patient an effective amount of the radioconjugated CCR8 targeting agent, such as but not limited to a radiolabeled anti-CCR8 monoclonal antibody or radiolabeled CCR8-binding fragment of a monoclonal antibody. The effective amount of the radioconjugated CCR8 targeting agent may, for example, be a maximum tolerated dose (MTD) or may be a fractioned dose wherein the total amount of radiation administered in the fractioned doses is the MTD.

The radioconjugated CCR8 targeting agent may, for example, include a radiolabeled fraction and a non-radiolabeled fraction. As such, an effective amount of the radioconjugated CCR8 targeting agent may, for example, include a total protein dose of less than 100 mg, such as from 5 mg to 60 mg, or 5 mg to 45 mg. The total protein dose may, for example, be from 0.001 mg/kg to 3 mg/kg body weight of the subject, such as from 0.005 mg/kg to 2 mg/kg body weight of the subject. The total protein dose may, for example, be at or less than 2 mg/kg, or at or less than 1 mg/kg, or at or less than 0.5 mg/kg, or at or less than 0.1 mg/kg.

An effective amount of a radioconjugated CCR8 targeting agent, such as an mAc-anti-CCR8 antibody, peptide, or small molecule, may, for example, include a radiation dose of 0.1 to 50 μCi/kg body weight of the subject, such as 0.1 to 5 μCi/kg body weight of the subject, or 5 to 20 μCi/kg subject body weight, or a radiation dose of 2 μCi to 2mCi, or 2 μCi to 250 μCi, or 75 Ci to 400 μCi in a non-weight-based radiation dose.

An effective amount of a radioconjugated CCR8 targeting agent, such as an ¹⁷⁷Lu-anti-CCR8 antibody, peptide, or small molecule, may for example, include a radiation dose of 1 to 1000 μCi/kg body weight of the subject, such as 5 to 250 μCi/kg body weight of the subject, or 50 to 450 μCi/kg body weight, or a radiation dose of 10 mCi to 30 mCi, or 100 μCi to 3 mCi, or 3 mCi to 30 mCi in a non-weight-based radiation dose.

An effective amount of a radioconjugated CCR8 targeting agent, such as an 131I-anti-CCR8 antibody, peptide, or small molecule, may, for example, include a dose of at or below 1200 mCi in a non-weight-based radiation dose, such as from at least 1 mCi to at or below 100 mCi, or at least 10 mCi to at or below 200 mCi.

The effective amount of the radioconjugated CCR8 targeting agent, may depend on the configuration of the targeting agent, i.e., full length antibody or antibody fragment (e.g., minibody, nanobody, etc). For example, when the CCR8 targeting agent includes an ²²⁵Ac-anti-CCR8 targeting agent that is a full-length antibody, the dose may, for example, be at or below 5 μCi/kg body weight of the subject, such as 0.1 to 5 μCi/kg body weight of the subject. Alternatively, when the CCR8 targeting agent includes an ²²⁵Ac-anti-CCR8 targeting agent that is a fragment, the dose may, for example, be greater than 5 μCi/kg body weight of the subject, such as 5 to 20 μCi/kg body weight of the subject.

In aspects of the invention in which more than one radiolabeled targeting agent, such as more than one radiolabeled antibody, is used, such as a radiolabeled anti-CCR8 antibody and a radiolabeled antibody against a different antigen, such as a cancer associated antigen, the radiation and/or protein doses of each radiolabeled targeting may, for example, be within the same ranges and amounts described herein for an anti-CCR8 targeting agent, or the total radiation and/or protein doses of all radiolabeled targeting agents employed may, for example, be within the same ranges and amounts described herein for a radiolabeled CCR8 targeting agent, such a radiolabeled anti-CCR8 antibody.

The radioconjugated CCR8 targeting agent may, for example, be administered according to a dosing schedule selected from the group consisting of one dose every 7, 10, 12, 14, 20, 24, 28, 35, and 42 days throughout a treatment period, wherein the treatment period includes at least two doses.

The radioconjugated CCR8 targeting agent may, for example, be administered according to a dose schedule that includes 2 doses, such as on days 1 and 5, 6, 7, 8, 9, or 10 of a treatment period, or days 1 and 8 of a treatment period.

The radioconjugated CCR8 targeting agent may, for example, be administered as a single bolus or infusion.

Each administration of the radioconjugated CCR8 targeting agent may, for example, be of a subject-specific dose, wherein each of a protein dose and a radiation dose are selected based on subject specific characteristics (e.g., weight, age, gender, health status, and/or nature and severity of the cancer or tumor, etc.).

The treatment methods including administration of a radiolabeled CCR8 targeting agent may further include administration of one or more further therapeutic agents, such as a chemotherapeutic agent, an anti-inflammatory agent, an immunosuppressive agent, an immunomodulatory agent, an antimyeloma agent, a cytokine, or any combination thereof. Exemplary chemotherapeutic agents include at least radiosensitizers that may synergize with the radiolabeled CCR8, such as temozolomide, cisplatin, and/or fluorouracil.

The treatment methods including administration of a radiolabeled CCR8 targeting agent may further include administration of one or more immune checkpoint therapies. Exemplary immune checkpoint therapies that may be used include an antibody against CTLA-4, PD-1, TIM3, VISTA, BTLA, LAG-3, TIGIT, CD28, OX40, GITR, CD137, CD40, CD40L, CD27, HVEM, PD-L1, PD-L2, PD-L3, PD-L4, CD80, CD86, CD137-L, GITR-L, CD226, B7-H3, B7-H4, BTLA, TIGIT, GALS, KIR, 2B4, CD160, CGEN-15049, or any combination thereof. The immune checkpoint therapy may include an antibody against an immune checkpoint protein selected from the group consisting of an antibody against PD-1, PD-L1, CTLA-4, TIM3, LAG3, VISTA, and any combination thereof. The immune checkpoint therapy may, for example, be provided in a subject effective amount including a dose of 0.1 mg/kg to 50 mg/kg of the patient's body weight, such as 0.1-5 mg/kg, or 5-30 mg/kg.

The treatment methods including administration of a radiolabeled CCR8 targeting agent may further include administration of one or more DNA damage response inhibitors (DDRi). Exemplary DDRi's that may be used include at least one or more antibodies or small molecules targeting poly(ADP-ribose) polymerase (i.e., a poly(ADP-ribose) polymerase inhibitor or PARPi). The PARPi may be a small molecule therapeutic selected from the group consisting of olaparib, niraparib, rucaparib, talazoparib, and any combination thereof. The PARPi may be provided in a subject effective amount including 0.1 mg/day-1200 mg/day, such as 0.100 mg/day-600 mg/day, or 0.25 mg/day-1 mg/day. Exemplary subject effective amounts include 0.1 mg, 0.25 mg, 0.5 mg, 0.75 mg, 1.0 mg, 100 mg, 200 mg, 300 mg, 400 mg, 500 mg, 600 mg, 700 mg, 750 mg, 800 mg, 900 mg, and 1000 mg, taken orally in one or two doses per day. Another exemplary DDRi that may be used includes an inhibitor of Ataxia telangiectasia mutated (ATM), Ataxia talangiectasia mutated and Rad-3 related (ATR), or Wee1. Exemplary inhibitors of ATM that may be used include KU-55933, KU-59403, wortmannin, CP466722, and KU-60019. Exemplary inhibitors of ATR include at least Schisandrin B, NU6027, NVP-BEA235, VE-821, VE-822, AZ20, and AZD6738. Exemplary inhibitors of Wee1 include AZD-1775 (i.e., adavosertib).

The treatment methods including administration of a radiolabeled CCR8 targeting agent may further include administration of one or more CD47 blockades. The CD47 blockade may, for example, include a monoclonal antibody or fusion protein that prevents CD47 binding to SIRPα. Specific CD47 blockades that may be used include magrolimab, lemzoparlimab, AO-176, AK117, IMC-002, IBI-188, IBI-322, BI 766063, ZL-1201, AXL148, RRx-001, ES004, SRF231, SHR-1603, TJC4, TTI-621, TTI-622, ALX148, and RRx-001. Exemplary effective doses for the CD47 blockade include 0.05 to 5 mg/kg patient weight. The CD47 blockade may also include agents that modulate the expression of CD47 and/or SIRPα, such as a nucleic acid approach, such as phosphorodiamidate morpholino oligomers (PMO) that block translation of CD47, such as MBT-001.

The treatment methods including administration of a radiolabeled CCR8 targeting agent may further include administration of a therapeutic that binds to MHC class I chain-related molecule A (MICA) to allow stabilization of surface expression of MICA on cancer cells for enhanced NK cell-mediated cytolysis. Exemplary anti-MICA antibodies that may be used include at least clone IPH43 or HYB3-24302 from Creative Biolabs, or anti-MICA antibodies against the alpha-3 domain, such as clone 7C6 reported in U.S. Pub. No. 20200165343.

The treatment methods including administration of a radiolabeled CCR8 targeting agent may further include administration of a therapeutic modality such as radiation therapy. Exemplary radiation therapies include external beam radiation and brachytherapy.

The treatment methods including administration of a radiolabeled CCR8 targeting agent may further include administration of any combination of the further therapeutic agents or modalities. Exemplary combinations include at least one or more DDRi, and/or one or more immune checkpoint therapies, and/or one or more CD47 blockades, and/or one or more therapeutics that bind to MICA, and/or one or more chemotherapeutics, and/or one or more radiation therapies (e.g., external beam radiation or brachytherapy).

The radioconjugated CCR8 targeting agent and the one or more further therapeutic agents or modalities may be administered simultaneously or sequentially. When more than one additional therapeutic agent and/or modality is administered, the agents may be administered simultaneously or sequentially.

The radioconjugated CCR8 targeting agent may, for example, be a portion of a multi-specific antibody. Thus, the methods may include administering to the subject an effective amount of a multi-specific antibody, wherein the multi-specific antibody includes: a first target recognition component which specifically binds to an epitope of CCR8, and a second target recognition component which specifically binds to a different epitope of CCR8 than the first target recognition component, or to an epitope of a different antigen, such as a cancer-associated antigen or immune suppressor cell associated antigen. The CCR8 targeting agent may, for example, be a multi-specific antibody against a first epitope of CCR8 and at least a second epitope of CCR8, or against CCR8 and at least a second antigen, such as a cancer-associated antigen or immune suppressor cell associated antigen.

Additional features, advantages, and embodiments of the invention may be set forth or apparent from consideration of the following detailed description, drawings if any, and claims. Moreover, it is to be understood that both the foregoing summary of the invention and the following detailed description are exemplary and intended to provide further explanation without limiting the scope of the invention as claimed.

### DETAILED DESCRIPTION OF THE INVENTION

The present disclosure provides methods and compositions for treating cancer by depleting tumor-infiltrating/-resident Treg cells. Specifically, the present disclosure relates to radioconjugated (radiolabeled) CCR8 targeting agents that may be exploited for Treg depletion or ablation from the tumor microenvironment and subsequent re-invigoration of antitumor immunity. While multiple monoclonal antibodies have been described in the literature that target CCR8 to cause immunodepletion of Tregs in preclinical models, no clinical-stage therapeutic exists yet. Moreover, to date, each of the CCR8 targeting agents have been payload-free antibodies that rely on NK cell recruitment for ADCC-mediated Treg depletion.

As an alternative to ADCC, targeting agents may be conjugated with other moieties that confer additional effects. For instance, antibody-drug conjugates (ADCs) allow targeted delivery of chemotherapeutic agents to cells that express the antibody target (Drago, 2021). Thus, CCR8 targeting agents could be conjugated with drugs to allow more efficient eradication of tumor infiltrating Tregs. Despite the seemingly simple mechanism of action of ADCs, potential limitations to this approach include the requirement for high target density, ADC internalization, trafficking of the ADC to the correct intracellular compartment, and release of sufficient quantities of the drug payload (Drago, 2021). It is unclear if the surface antigen level of CCR8 on tumor-associated Tregs is sufficient to enable a cytotoxic effect from an ADC approach.

Antibody radio-conjugates (ARCs) that utilize potent radionuclides for targeted cell ablation are ideal alternatives to ADCs and ADCC-dependent mechanisms. Thus far, radioconjugated targeting agents have typically been used to kill malignant cells that express certain tumor antigens (e.g., SSTR2, CD20, PSMA; Nelson, 2020). The present disclosure utilized radioimmunotherapy to target and deplete tumor infiltrating Tregs by targeting CCR8 with radioconjugated CCR8 targeting agents.

The potency of ionizing radiation in the present compositions and methods causes lethal DNA damage. Thus, cell surface density of the CCR8 target is less of a factor compared to ADCs, as a single alpha particle can cause cell death through clusters of dsDNA breaks (Nelson, 2020; Neti, 2006). Moreover, radiation is deliverable from the surface of the cell after the ARC binds to the target CCR8, obviating the need for internalization.

ARCs are also less sensitive to tumor “coldness”. For example, while NK cells are able to kill CCR8-expressing Tregs through ADCC, NK cells are negatively affected by an immunosuppressive environment (Melaiu, 2020), which may diminish the magnitude of Treg cell depletion. In contrast, because ARCs do not rely on a cellular intermediary to carry out the cytotoxic function, ARCs can kill Tregs irrespective of whether the tumor microenvironment is “hot” or “cold”. Tumor infiltrating Tregs are detrimental to patients with multiple cancer types, and the radioconjugated CCR8 targeting agents disclosed herein may deplete Tregs more effectively than biologics currently in development.

The tumor-selective presence of CCR8+ Treg cells (De Simone, 2016; Plitas, 2016) can be leveraged for another beneficial feature of radioimmunotherapy, the “crossfire effect.” The crossfire effect refers to the delivery of ionizing radiation to cells adjacent to a target cell to which a radiolabeled targeting agent has bound (Haberkorn, 2017). In this way, radioimmunotherapy may have a distinct advantage over antibody conjugation approaches that may exhibit reduced efficacy when tumor antigen expression is heterogeneous within the diseased tissue. In the case of ADCs, this is because the drug payload cannot internalize to an adequate extent into cells that have little-to-no target expression. In contrast, radiation from a radioisotope can hit multiple cells within the vicinity of the initially targeted cell. Advantageously, in the case of targeted radioimmunotherapy against tumor-infiltrating Tregs, a radioconjugated CCR8-targeting can simultaneously ablate the immunosuppressive Tregs and cancerous cells surrounding the targeted Tregs.

The crossfire effect is a unique feature of radioimmunotherapy that cannot be replicated by other targeted therapeutic approaches. Accordingly, greater clinical efficacy compared to currently investigated treatment modalities that target CCR8 may be realized. Since CCR8 is highly expressed only on tumor-infiltrating Tregs (and not peripheral Tregs; De Simone, 2016; Plitas, 2016), the crossfire killing would be tumor-specific, and the risk of toxicity to healthy tissue would be minimal. To this end, while both alpha and beta emitting radioisotopes would be effective in targeting both CCR8+ Tregs and adjacent tumor tissue through crossfire, the use of an alpha emitting radioisotope such as Actinium-225 may have a further advantage in limiting damage to surrounding normal tissue outside the TME due to the short path length (3-4 cell lengths) its high energy alpha particle will travel.

The radioconjugated CCR8 targeting approach possesses another advantage over a non-radioconjugated targeted approach: immunostimulation. Radiation is known to stimulate the immune response in the tumor microenvironment (Dar, 2019; Sharabi, 2015). For instance, external radiation leads to the release of danger-associated molecular patterns (DAMPs) that cause innate immune cell recruitment and subsequent release of pro-inflammatory cytokines (e.g., TNF a, type I interferon, IL-1β). Afterwards, these innate immune cells present antigen to adaptive immune cells in lymphoid tissue, activating effector T cell response against the tumor (Dar, 2019). Likewise, internal delivery of targeted ionizing radiation may lead to similar effects as treatment with external radiation. By causing the release of DAMPs in the tumor microenvironment—using CCR8-expressing Tregs as homing beacons for radiation deposition—radioimmunotherapy may contribute to antitumor immune response that is greater in magnitude compared to what could be accomplished by standard Treg depletion. Due to this ancillary immunostimulatory effect of radiation, the presently disclosed radio-conjugate approach may synergize more efficiently with immunomodulatory therapy (e.g., anti-PD-(L)1, anti-CTLA-4) compared to an unconjugated approach.

Radiation also causes immunogenic cell death and, of particular importance, cell surface exposure of calreticulin leads to macrophage-mediated phagocytosis of the cancer cell (Wang, 2019, Clin Cancer Res). Although radiation induces calreticulin exposure, CD47 co-expression on cancer cells inhibits phagocytosis (Takimoto, 2019). Thus, CD47 blockade is another immunomodulatory avenue that may synergize with radio-immunotherapy. In stark contrast to radioconjugates, CCR8-targeting approaches that rely solely on ADCC (or drug conjugates) would not elicit immunostimulation in the tumor microenvironment to the same extent because only the targeted cell—in this case, Treg cell—would be silently ablated.

For the preceding reasons—potency of the radionuclide payload, lack of requirement for drug internalization, crossfire effect against cancer cells, and immunostimulation within the tumor microenvironment—the presently disclosed radioconjugate approach provides a superior therapy to drug conjugates or naked antibodies (even if enhanced for ADCC). Consequently, lower amounts of total antibody and/or targeting agent may, for example, be used to achieve clinical efficacy.

Accordingly, the present disclosure provides novel methods and compositions for treating cancer by depleting tumor infiltrating Treg cells. Specifically, the present disclosure relates to radiolabeled CCR8 targeting agents that can achieve specific depletion of tumor infiltrating Treg cells with little influence on Treg cells in healthy tissues and/or effector T cells. The methods generally include administering to the patient an effective amount of a radioconjugated CCR8 targeting agent, such as a radiolabeled antibody, peptide, or small molecule targeted to CCR8, alone or in combination with one or more additional therapeutic agents or modalities.

The additional therapeutic agents include at least one or more immune checkpoint therapies and/or one or more MICA blockades and/or one or more inhibitors of a component of the DNA damage response pathway (i.e., a DNA damage response inhibitor, DDRi, such as one or more agents against poly(ADP-ribose) polymerase, i.e., PARPi) and/or one or more CD47/SIRPα axis blockades and/or one or more chemotherapeutic agents such as radiosensitizers. Additional therapeutic agents and modalities may include an anti-inflammatory agent, an immunosuppressive agent, an immunomodulatory agent, an antimyeloma agent, a cytokine, external beam radiation, or any combination thereof.

The present disclosure further provides methods for diagnosing patients having CCR8-positive tumor infiltrating Treg cells, followed by treating those patients according to any of the methods disclosed herein.

Prior to setting forth the invention in greater detail, it may be helpful to an understanding thereof to set forth definitions of certain terms to be used hereinafter.

### Definitions and Abbreviations

The singular forms “a,” “an,” “the” and the like include plural referents unless the context clearly dictates otherwise. Thus, for example, reference to “an” antibody includes both a single antibody and a plurality of different antibodies.

The term “about” when used before a numerical designation, e.g., temperature, time, amount, and concentration, including a range, indicates approximations which may vary by ±10%, ±5%, or ±1%.

As used herein, “administer”, with respect to a targeting agent such as an antibody, antibody fragment, Fab fragment, or aptamer, means to deliver the agent to a subject's body via any known method suitable for antibody delivery. Specific modes of administration include, without limitation, intravenous, transdermal, subcutaneous, intraperitoneal, intrathecal and intra-tumoral administration. Exemplary administration methods for antibodies may be as substantially described in U.S. Pat. No. 10,736,975 or International Pub. No. WO2016187514, each incorporated by reference herein.

In addition, in this disclosure, antibodies can be formulated using one or more routinely used pharmaceutically acceptable carriers. Such carriers are well known to those skilled in the art. For example, injectable drug delivery systems include solutions, suspensions, gels, microspheres and polymeric injectables, and can include excipients such as solubility-altering agents (e.g., ethanol, propylene glycol and sucrose) and polymers (e.g., polycaprylactones and PLGA's).

As used herein, the term “antibody” includes, without limitation, (a) an immunoglobulin molecule including two heavy chains and two light chains and which recognizes an antigen; (b) polyclonal and monoclonal immunoglobulin molecules; (c) monovalent and divalent fragments thereof, such as Fab, di-Fab, scFvs, diabodies, minibodies, nanobodies, and single domain antibodies (sdAb); (d) naturally occurring and non-naturally occurring, such as wholly synthetic antibodies, IgG-Fc-silent, and chimeric; and (e) bi-specific forms thereof. Immunoglobulin molecules may derive from any of the commonly known classes, including but not limited to IgA, secretory IgA, IgG and IgM. IgG subclasses are also well known to those in the art and include, but are not limited to, human IgG1, IgG2, IgG3 and IgG4. The N-terminus of each chain defines a “variable region” of about 100 to 110 or more amino acids primarily responsible for antigen recognition. The terms variable light chain (VL) and variable heavy chain (VH) refer to these regions of light and heavy chains respectively. Antibodies may be human, humanized or nonhuman. When a specific aspect of the present disclosure refers to or recites an “antibody,” it is envisioned as referring to any of the full-length antibodies or fragments thereof disclosed herein, unless explicitly denoted otherwise.

A “humanized” antibody refers to an antibody in which some, most or all amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

A “complementarity-determining region”, or “CDR”, refers to amino acid sequences that, together, define the binding affinity and specificity of the variable region of a native immunoglobulin binding site. There are three CDRs in each of the light and heavy chains of an antibody. CDRs may, for example, be delineated according to the Kabat or IMGT numbering conventions.

A “framework region”, or “FR”, refers to amino acid sequences interposed between CDRs, typically conserved, that act as the scaffold between the CDRs.

A “constant region” refers to the portion of an antibody molecule that is consistent for a class of antibodies and is defined by the type of light and heavy chains. For example, a light chain constant region can be of the kappa or lambda chain type and a heavy chain constant region can be of one of the five chain isotypes: alpha, delta, epsilon, gamma or mu. This constant region, in general, can confer effector functions exhibited by the antibodies. Heavy chain constant regions of various subclasses (such as the IgG subclass of heavy chains) are mainly responsible for different effector functions.

As used herein, a “CCR8 targeting agent” may, for example, be an antibody as defined herein, e.g., full length antibody, CCR8-binding antibody fragment such as Fab, Fab₂ or scFv molecule, minibody, single domain antibody, nanobody, etc., that binds to any available epitope of CCR8, such as of the extracellular domain, with a high immunoreactivity. Exemplary antibodies that may be radiolabeled and used in the methods of the present disclosure may include a rat anti-human CCR8 recombinant antibody such as clone CBL712 from Creative Biolabs®, Shirley, New York, USA (CAT #: NEUT-274CQ), or a mouse anti-human CCR8 recombinant antibody also from Creative Biolabs® (CAT #: MOB-3049z), or the anti-mouse CD198 (CCR8) antibody clone SA214G2 from BioLegend® (San Diego, California, USA), or antibody clone 10A11 or any of the other anti-CCR8 antibodies disclosed in Taiwanese Patent Application TW202039575A, Australian Patent Application AU2019415395 or Int'l Pub. No. WO2020138489, or antibodies including the heavy chain variable region and/or the light chain variable region of any of said antibodies, or antibodies including the heavy chain CDRs and/or the light chain CDRs of any of said antibodies.

The CCR8 targeting agent may, for example, be an antibody or a CCR8 binding fragment thereof, such as an IgG or antigen-binding fragment thereof, including a light chain CDR1 having the amino acid sequence RSSKSLLHSNGNTYLY (SEQ ID NO:173), a light chain CDR2 having the amino acid sequence RMSNLAS (SEQ ID NO:174), and a light chain CDR3 having the amino acid sequence MQHLEYPLT (SEQ ID NO:175) and/or the CCR8 targeting agent may be an antibody including a heavy chain CDR1 having the amino acid sequence TYALY (SEQ ID NO:176), a heavy chain CDR2 having the amino acid sequence RIRSKSNNYATYYADSVKD (SEQ ID NO:177), and a heavy chain CDR3 having the amino acid sequence ARFYYSDYGYAMDY (SEQ ID NO:178). Any combination of light chain CDR sequences and/or heavy chain CDR sequences listed herein while maintaining antibody CCR8 binding may be used and is within the scope of the present disclosure.

Moreover, certain isomeric amino acid replacements with exact mass, such as Leu for Ile or vice versa, could be allowed in any of the sequences indicated herein. Additionally, certain portions of these sequences may be substituted, such as by related portions from human immunoglobulins to form chimeric immunoglobulins (i.e., chimeric or humanized CCR8). Exemplary substitutions include all or portions of the human leader sequence, and/or the conserved regions from human IgG1, IgG2, or IgG4 heavy chains and/or human Kappa light chain.

The radiolabeled CCR8 targeting agent may, for example, be a multi-specific antibody against a first epitope of CCR8 and at least a second epitope of CCR8, or against CCR8 and at least a second antigen.

The radiolabeled CCR8 targeting agent may, for example, be a component of a mixture of a radiolabeled antibody against an epitope of CCR8 and one or more antibodies against a different epitope of CCR8 which may also be radiolabeled.

The radiolabeled CCR8 targeting agent may, for example, be a peptide or small molecule that binds to CCR8.

As used herein, “Immunoreactivity” refers to a measure of the ability of an immunoglobulin to recognize and bind to a specific antigen. “Specific binding” or “specifically binds” or “binds” refers to an antibody binding to an antigen or an epitope within the antigen with greater affinity than for other antigens within the relevant milieu, such as within the tissues of the treatment subject. Typically, an antibody may bind to the antigen or the epitope within the antigen with an equilibrium dissociation constant (K_{D}) of about 1×10⁻⁸M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a nonspecific antigen (e.g., BSA, casein). The dissociation constant may be measured using standard procedures. Antibodies that specifically bind to the antigen or the epitope within the antigen may, however, have cross-reactivity to other related antigens, for example to the same antigen from other species (homologs), such as human or monkey, for example Macaca fascicularis (cynomolgus, cyno), Pan troglodytes (chimpanzee, chimp) or Callithrix jacchus (common marmoset, marmoset).

An “epitope” refers to the target molecule site (e.g., at least a portion of an antigen) that is capable of being recognized by, and bound by, a targeting agent such as an antibody, antibody fragment, Fab fragment, or aptamer. For a protein antigen, for example, this may refer to the region of the protein (i.e., amino acids, and particularly their side chains) that is bound by the antibody. Overlapping epitopes include at least one to five common amino acid residues. Methods of identifying epitopes of antibodies are known to those skilled in the art and include, for example, those described in Antibodies, A Laboratory Manual, Cold Spring Harbor Laboratory, Ed Harlow and David Lane (1988).

As used herein, the terms “proliferative disorder” and “cancer” may be used interchangeably and may include, without limitation, solid cancers (e.g., solid tumors) and hematological cancers. “Solid cancers” include, without limitation, bone cancer, pancreatic cancer, skin cancer, cancer of the head or neck, cutaneous or intraocular malignant melanoma, uterine cancer, ovarian cancer, prostate cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, pediatric tumors, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally-induced cancers including those induced by asbestos. Such cancers may be treated by the compositions and methods disclosed herein.

The solid cancer may be breast cancer, gastric cancer, bladder cancer, cervical cancer, endometrial cancer, skin cancer, stomach cancer, testicular cancer, esophageal cancer, bronchioloalveolar cancer, prostate cancer, colorectal cancer, ovarian cancer, cervical epidermoid cancer, pancreatic cancer, lung cancer such as non-small cell lung carcinoma, renal cancer, head and neck cancer such as head and neck squamous cell cancer, or any combination thereof. Such cancers may be treated by the compositions and methods disclosed herein.

The CCR8 targeting agent may be labeled with a radioisotope. As used herein, a “radioisotope” can be an alpha-emitting isotope, a beta-emitting isotope, and/or a gamma-emitting isotope. As used herein, the term “radioisotope” is synonymous with “radionuclide”. Examples of radioisotopes that may be used to label a CCR8 targeting agent or other targeting agent include the following: ¹³¹I, ¹²⁵I, ¹²³I, ⁹⁰Y, ¹⁷⁷Lu, ¹⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb and ¹⁰³Pd. Methods for affixing a radiolabel to a protein such as an antibody or antibody fragment (i.e., “labeling” an antibody with a radioisotope) are well known. Suitable methods for radiolabeling are described, for example, in U.S. Pat. Nos. 10,420,851, 9,603,954, Int'l Pub. No. WO 2017155937 and U.S. Provisional Patent Application No. 63/119,093, filed Nov. 30, 2020 and titled “Compositions and methods for preparation of site-specific radioconjugates,” all of which are incorporated by reference herein.

The terms “radiolabeled” and “radioconjugated” are used interchangeably herein in describing a targeting agent that includes a radioisotope/radionuclide affixed thereto, for example, either covalently or non-covalently, such as via chelation by a chelator.

The CCR8 targeting agent may, for example, be an antibody, CCR8-binding antibody fragment, peptide, or small molecule radiolabeled with ²²⁵Ac (“²²⁵Ac-labeled” or radioconjugated CCR8-targeting agent), and the effective amount may be, for example, at or below 50.0 μCi/kg (i.e., where the amount of ²²⁵Ac administered to the subject delivers a radiation dose of at or below 50.0 μCi per kilogram of subject's body weight). When the CCR8 targeting agent is ²²⁵Ac-labeled, the effective amount is at or below 50 μCi/kg, 40 μCi/kg, 30 μCi/kg, 20 μCi/kg, 10 μCi/kg, 5 μCi/kg, 4 μCi/kg, 3 μCi/kg, 2 μCi/kg, 1 μCi/kg, or even 0.5 μCi/kg. When the CCR8 targeting agent is ²²⁵Ac-labeled, the effective amount is at least 0.05 μCi/kg, or 0.1 μCi/kg, 0.2 μCi/kg, 0.3 μCi/kg, 0.4 μCi/kg, 0.5 μCi/kg, 1 μCi/kg, 2 μCi/kg, 3 μCi/kg, 4 μCi/kg, 5 μCi/kg, 6 μCi/kg, 7 μCi/kg, 8 μCi/kg, 9 μCi/kg, 10 μCi/kg, 12 μCi/kg, 14 μCi/kg, 15 μCi/kg, 16 μCi/kg, 18 μCi/kg, 20 μCi/kg, 30 μCi/kg, or 40 μCi/kg. The ²²⁵Ac-labeled targeting agent may, for example, be administered at a dose that includes any combination of upper and lower limits as described herein, such as from at least 0.1 μCi/kg to at or below 5 μCi/kg, or from at least 5 μCi/kg to at or below 20 μCi/kg.

The CCR8 targeting agent may, for example, be an antibody, CCR8-binding antibody fragment, peptide, or small molecule that is ²²⁵Ac-labeled, and the effective amount may be at or below 2 mCi (i.e., wherein the ²²⁵Ac is administered to the subject in a non-weight-based dosage). The effective dose of the ²²⁵Ac-labeled CCR8 targeting agent may be at or below 1 mCi, such as 0.9 mCi, 0.8 mCi, 0.7 mCi, 0.6 mCi, 0.5 mCi, 0.4 mCi, 0.3 mCi, 0.2 mCi, 0.1 mCi, 90 μCi, 80 μCi, 70 μCi, 60 μCi, 50 μCi, 40 μCi, 30 μCi, 20 μCi, 10 μCi, or 5 μCi. The effective amount of ²²⁵Ac-labeled CCR8 targeting agent may be at least 2 μCi, such as at least 5 μCi, 10 μCi, 20 μCi, 30 μCi, 40 μCi, 50 μCi, 60 μCi, 70 μCi, 80 μCi, 90 μCi, 100 μCi, 200 μCi, 300 μCi, 400 μCi, 500 μCi, 600 μCi, 700 μCi, 800 μCi, 900 μCi, 1 mCi, 1.1 mCi, 1.2 mCi, 1.3 mCi, 1.4 mCi, or 1.5 mCi. The ²²⁵Ac-labeled CCR8 targeting agent may, for example, be administered at a dose that includes any combination of upper and lower limits as described herein, such as from at least 2 Ci to at or below 1mCi, or from at least 2 μCi to at or below 250 μCi, or from 75 μCi to at or below 400 μCi.

The ²²⁵Ac-labeled CCR8 targeting agent may, for example, include and/or be formulated as a single dose that delivers less than 12Gy, or less than 8 Gy, or less than 6 Gy, or less than 4 Gy, or less than 2 Gy, such as doses of 2 Gy to 8 Gy, to the subject, such as predominantly to the targeted solid tumor.

The CCR8 targeting agent may, for example, be an antibody, CCR8-binding antibody fragment, peptide, or small molecule radiolabeled with ¹⁷⁷Lu (“¹⁷⁷Lu-labeled”), and the effective amount may be, for example, be at or below 1 mCi/kg (i.e., where the amount of ¹⁷⁷Lu-labeled antibody administered to the subject delivers a radiation dose of at or below 1000 mCi per kilogram of subject's body weight). When the targeting agent is ¹⁷⁷Lu-labeled, the effective amount is at or below 900 μCi/kg, 800 μCi/kg, 700 μCi/kg, 600 μCi/kg, 500 μCi/kg, 400 μCi/kg, 300 μCi/kg, 200 μCi/kg, 150 μCi/kg, 100 μCi/kg, 80 μCi/kg, 60 μCi/kg, 50 μCi/kg, 40 μCi/kg, 30 μCi/kg, 20 μCi/kg, 10 μCi/kg, 5 μCi/kg, or 1 μCi/kg. The effective amount of the ¹⁷⁷Lu-labeled targeting agent may be at least 1 μCi/kg, 2.5 μCi/kg, 5 μCi/kg, 10 μCi/kg, 20 μCi/kg, 30 μCi/kg, 40 μCi/kg, 50 μCi/kg, 60 μCi/kg, 70 μCi/kg, 80 μCi/kg, 90 μCi/kg, 100 μCi/kg, 150 μCi/kg, 200 μCi/kg, 250 μCi/kg, 300 μCi/kg, 350 μCi/kg, 400 μCi/kg or 450 μCi/kg. The ¹⁷⁷Lu-labeled targeting agent may, for example, be administered at a dose that includes any combination of upper and lower limits as described herein, such as from at least 5 mCi/kg to at or below 50 μCi/kg, or from at least 50 mCi/kg to at or below 500 μCi/kg.

The CCR8 targeting agent may, for example, be antibody, CCR8-binding antibody fragment peptide, or small molecule that is ¹⁷⁷Lu-labeled, and the effective amount may be at or below 45 mCi, such as at or below 40 mCi, 30 mCi, 20 mCi, 10 mCi, 5 mCi, 3.0 mCi, 2.0 mCi, 1.0 mCi, 800 μCi, 600 μCi, 400 μCi, 200 μCi, 100 μCi, or 50 μCi. The effective amount of ¹⁷⁷Lu-labeled CCR8 targeting agent may be at least 10 μCi, such as at least 25 μCi, 50 μCi, 100 μCi, 200 μCi, 300 μCi, 400 μCi, 500 μCi, 600 μCi, 700 μCi, 800 μCi, 900 μCi, 1 mCi, 2 mCi, 3 mCi, 4 mCi, 5 mCi, 10 mCi, 15 mCi, 20 mCi, 25 mCi, 30 mCi. The ¹⁷⁷Lu-labeled targeting agent may, for example, be administered at a dose that includes any combination of upper and lower limits as described herein, such as from at least 10 mCi to at or below 30 mCi, or from at least 100 μCi to at or below 3 mCi, or from 3 mCi to at or below 30 mCi.

The CCR8 targeting agent may, for example, be an antibody, CCR8-binding antibody fragment peptide, or small molecule radiolabeled with ¹³¹I (“¹³¹I-labeled”), and the effective amount may be at or below, for example, 1200 mCi (i.e., where the amount of ¹³¹I administered to the subject delivers a total body radiation dose of at or below 1200 mCi in a non-weight-based dose). The effective amount of the ¹³¹I-labeled targeting agent may, for example, be at or below 1100 mCi, at or below 1000 mCi, at or below 900 mCi, at or below 800 mCi, at or below 700 mCi, at or below 600 mCi, at or below 500 mCi, at or below 400 mCi, at or below 300 mCi, at or below 200 mCi, at or below 150 mCi, or at or below 100 mCi. The effective amount of the ¹³¹I-labeled targeting agent may, for example, be at or below 200 mCi, such as at or below 190 mCi, 180 mCi, 170 mCi, 160 mCi, 150 mCi, 140 mCi, 130 mCi, 120 mCi, 110 mCi, 100 mCi, 90 mCi, 80 mCi, 70 mCi, 60 mCi, or 50 mCi. The effective amount of the ¹³¹I-labeled targeting agent may, for example, be at least 1 mCi, such as at least 2 mCi, 3 mCi, 4 mCi, 5 mCi, 6 mCi, 7 mCi, 8 mCi, 9 mCi, 10 mCi, 20 mCi, 30 mCi, 40 mCi, 50 mCi, 60 mCi, 70 mCi, 80 mCi, 90 mCi, 100 mCi, 110 mCi, 120 mCi, 130 mCi, 140 mCi, 150 mCi, 160 mCi, 170 mCi, 180 mCi, 190 mCi, 200 mCi, 250 mCi, 300 mCi, 350 mCi, 400 mCi, 450 mCi, 500 mCi. An ¹³¹I-labeled targeting agent may, for example, be administered at a dose that includes any combination of upper and lower limits as described herein, such as from at least 1 mCi to at or below 100 mCi, or at least 10 mCi to at or below 200 mCi.

While select radionuclides have been disclosed in detail herein, any suitable radionuclides such as any of those disclosed herein may be used for labeling CCR8 targeting agents and other targeting agents in the various aspects of the invention.

As used herein, a composition including a CCR8 targeting agent may, for example, include both a radionuclide labeled portion and a non-radionuclide labeled portion. Such a composition may, for example, be a patient-specific composition tailored/formulated for a specific patient and/or formulated in a single-dose to be administered in its entirety to the patient in one administration. The majority of the targeting agent (antibody, antibody fragment, etc.) administered to a patient may, for example, consist of non-radiolabeled targeting agent, with the minority being the radiolabeled targeting agent. The ratio of radiolabeled to non-radiolabeled targeting agent can be adjusted using known methods. Such a composition may, for example, include the CCR8 targeting agent in a ratio of radiolabeled: unlabeled CCR8 targeting agent of from about 0.01:10 to 1:1, such as 0.1:10 to 1:1 radiolabeled: unlabeled.

The CCR8 targeting agent may, for example, be provided in a total protein amount of up to 100 mg, such as up to 60 mg, such as 5 mg to 45 mg, or a total protein amount of from 0.001 mg/kg patient weight to 3.0 mg/kg patient weight, such as from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight.

This inventive combination of a radiolabeled fraction and a non-radiolabeled fraction of the antibody or other biologic delivery vehicle allows the composition to be tailored to a specific patient, wherein each of the radiation dose and the protein dose of the antibody or other biologic delivery vehicle are personalized to that patient based on at least one patient specific parameter. As such, each vial of the composition may be made for a specific patient, where the entire content of the vial is delivered to that patient in a single dose. When a treatment regime calls for multiple doses, each dose may be formulated as a patient specific dose in a vial to be administered to the patient as a “single dose” (i.e., full contents of the vial administered at one time). The subsequent dose may be formulated in a similar manner, such that each dose in the regime provides a patient specific dose in a single dose container. One of the advantages of the disclosed composition is that there will be no left-over radiation that would need to be discarded or handled by the medical personnel, e.g., no dilution, or other manipulation to obtain a dose for the patient. When provided in a single dose container, the container may simply be placed in-line in an infusion tubing set for infusion to the patient. Moreover, the volume can be standardized so that there is a greatly reduced possibility of medical error (i.e., delivery of an incorrect dose, as the entire volume of the composition is to be administered in one infusion).

Thus, the radiolabeled CCR8 targeting agent may be provided as a single dose composition tailored to a specific patient, wherein the amount of labeled and unlabeled CCR8 targeting agent in the composition may depend on one or more of a patient weight, age, gender, disease state and/or health status. The CCR8 targeting agent may, for example, be provided as a multi-dose therapeutic, wherein each dose in the treatment regime is provided as a patient specific composition. The patient-specific composition may, for example, include radiolabeled and unlabeled (non-radiolabeled) CCR8 targeting agent molecules, wherein the amounts of each depend on one or more of patient weight, age, gender, disease state, and/or health status.

As used herein, the terms “subject” and “patient” are interchangeable and include, without limitation, a mammal such as a human, a non-human primate, a dog, a cat, a horse, a sheep, a goat, a cow, a rabbit, a pig, a rat and a mouse. Where the subject is human, the subject can be of any age. For example, the subject can be 60 years or older, 65 or older, 70 or older, 75 or older, 80 or older, 85 or older, or 90 or older. Alternatively, the subject can be 50 years or younger, 45 or younger, 40 or younger, 35 or younger, 30 or younger, 25 or younger, or 20 or younger. For a human subject afflicted with cancer, the subject can be newly diagnosed, or relapsed and/or refractory, or in remission.

As used herein, “treating” a subject afflicted with a cancer shall include, without limitation, (i) slowing, stopping or reversing the cancer's progression, (ii) slowing, stopping or reversing the progression of the cancer's symptoms, (iii) reducing the likelihood of the cancer's recurrence, and/or (iv) reducing the likelihood that the cancer's symptoms will recur. According to certain preferred aspects, treating a subject afflicted with a cancer means (i) reversing the cancer's progression, ideally to the point of eliminating the cancer, and/or (ii) reversing the progression of the cancer's symptoms, ideally to the point of eliminating the symptoms, and/or (iii) reducing or eliminating the likelihood of relapse (i.e., consolidation, which ideally results in the destruction of any remaining cancer cells).

“Chemotherapeutic”, in the context of this disclosure, shall mean a chemical compound which inhibits or kills growing cells and which can be used or is approved for use in the treatment of cancer. Exemplary chemotherapeutic agents include cytostatic agents which prevent, disturb, disrupt or delay cell division at the level of nuclear division or cell plasma division. Such agents may stabilize microtubules, such as taxanes, in particular docetaxel or paclitaxel, and epothilones, in particular epothilone A, B, C, D, E, and F, or may destabilize microtubules such as vinca alkaloids, in particular vinblastine, vincristine, vindesine, vinflunine, and vinorelbine. Exemplary chemotherapeutics also include radiosensitizers that may synergize with a radiolabeled CCR8 targeting agent, such as temozolomide, cisplatin, and/or fluorouracil.

“Therapeutically effective amount” or “effective amount” refers to an amount effective, at dosages and for periods of time necessary, to achieve a desired therapeutic result, either alone or in combination with one or more other agents or treatment modalities. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body. According to certain aspects, “therapeutically effective amount” or “effective amount” refers to an amount of the radioconjugated CCR8 targeting agent that may deplete or cause a reduction in the overall number of cells expressing CCR8, or that may inhibit or slow the growth of tumors having CCR8 expressing cells therein, or reduce the overall tumor burden of tumors having CCR8-expressing cells therein (i.e., tumor infiltrating CCR8 expressing Treg cells).

As used herein, “depleting”, with respect to cells expressing CCR8, shall mean to lower the (living) population of at least one type of cells that express or overexpress CCR8 (e.g., CCR8-positive cells in a solid tumor such as the CCR8-expressing Treg cells located in the tumor micro-environment or TME). According to certain aspects of this disclosure, a decrease may be determined by comparison of the numbers of CCR8-positive cells (Tregs in the TME) in a tissue biopsy, such as from the solid tumor, before and after initiation of treatment with the CCR8 targeting agent. According to certain aspects of this disclosure, a decrease may also be determined by overall tumor size. As such, and by way of example, a subject's tumor size may, for example, be considered to be depleted if the population of tumor cells is lowered, such as by at least 10%, 20%, 30%, 40%, 50%, 60%, 70%, 80%, 90% or 99%.

“Inhibits growth” refers to a measurable decrease or delay in the growth of a malignant cell or tissue (e.g., tumor) in vitro or in vivo when contacted with a therapeutic or a combination of therapeutics or drugs, when compared to the decrease or delay in the growth of the same cells or tissue in the absence of the therapeutic or the combination of therapeutic drugs. Inhibition of growth of a malignant cell or tissue in vitro or in vivo may, for example, be at least about 10%, 20%, 30%, 40%, 50%, 60%, 70%, 80%, 90%, 95%, 96%, 97%, 98%, 99%, or 100%.

The term “antitumor immunity” refers to the ability of the presently disclosed compositions and methods to promote an antitumor effect by activating or promoting the activation of anti-tumor T cells and/or B cells. Such an antitumor effect may be confirmed through comparison of treated mice (i.e., treated with at least the radioconjugated CCR8 targeting agent disclosed herein) having normal immune functions and those with impaired immune functions, such as impaired T cells and B cells (nude mice). Alternatively, or additionally, antitumor immunity may be confirmed by determining the fraction of CD45-, CD3-, and CD8-positive cells (CD8-positive T cells) among living cells using flow cytometry, wherein increased numbers of CD45-, CD3-, and CD8-positive cells are expected for treated tumor-bearing mice as compared to untreated mice. Alternatively, the effect may be confirmed by analyzing images of an excised tumor stained with an anti-CD8 antibody and counting the number of CD8-positive cells per unit area in the tumor to examine the increased number of CD45-, CD3-, and CD8-positive cells for treated as compared to a untreated mice.

The term “immune checkpoint therapy” refers to a molecule capable of modulating the function of an immune checkpoint protein in a positive or negative way (for example, the interaction between an antigen presenting cell (APC) such as a cancer cell and an immune T effector cell). The term “immune checkpoint” refers to a protein directly or indirectly involved in an immune pathway that under normal physiological conditions is crucial for preventing uncontrolled immune reactions and thus for the maintenance of self-tolerance and/or tissue protection. The one or more immune checkpoint therapies described herein may independently act at any step of the T cell-mediated immunity including clonal selection of antigen-specific cells, T cell activation, proliferation, trafficking to sites of antigen and inflammation, execution of direct effector function and signaling through cytokines and membrane ligands. Each of these steps is regulated by counterbalancing stimulatory and inhibitory signals that fine tune the response.

In the context of the present disclosure, an immune checkpoint therapy encompasses therapies such as antibodies capable of down-regulating at least partially the function of an inhibitory immune checkpoint (antagonist) and/or up-regulating at least partially the function of a stimulatory immune checkpoint (agonist). For example, an immune checkpoint therapy may include an immune checkpoint inhibitor (ICI), such as a blocking antibody against an inhibitory immune checkpoint protein that is upregulated in certain cancers.

The term “DDRi” refers to an inhibitor of a DNA damage response pathway protein, of which a PARPi is an example. The term “PARPi” refers to an inhibitor of poly(ADP-ribose) polymerase. In the context of the present disclosure, the term PARPi encompasses molecules that may bind to and inhibit the function of poly(ADP-ribose) polymerase, such as antibodies, peptides, or small molecules.

The term “CD47 blockade” refers to an agent that prevents CD47 binding to SIRPα, such as agents that bind to either of CD47 or SIRPα, those that modulate expression of CD47 or SIRPα, or those that otherwise diminish the “don't eat me” activity of the CD47SIRPα axis. In the context of the present disclosure, CD47 blockades include at least antibodies that bind to CD47 such as magrolimab, lemzoparlimab, and AO-176, antibodies that bind to SIRPα, CD47-binding SIRPα Fc fusion proteins, agents that modulate the expression of CD47 and/or SIRPα, such as phosphorodiamidate morpholino oligomers (PMO) that block translation of CD47 such as MBT-001, and small molecule inhibitors of the CD47/SIRPα axis such as RRx-001.

The term “MICA blockade” refers to a molecule that targets, i.e., binds to and/or inhibits, the MHC class I chain-related molecule A (MICA), and provides stabilization of surface expression of MICA on cancer cells for enhanced NK cell-mediated cytolysis. Exemplary anti-MICA antibodies include at least clone IPH43 or HYB3-24302 from Creative Biolabs, or anti-MICA antibodies against the alpha-3 domain, such as clone 7C6 reported in U.S. Pub. No. 2020/0165343.

As used herein, administering to a subject one or more additional therapies, such as one or more of an immune checkpoint therapy and/or MICA blockade and/or DDRi and/or CD47 blockade and/or radiosensitizer and/or anon-CCR8 targeting agent “in combination with” or “in conjunction with” a radioconjugated CCR8 targeting agent means administering the additional therapy before, during and/or after administration of the radioconjugated CCR8 targeting agent. This administration includes, without limitation, the following scenarios: (i) the additional therapy is administered first, and the radioconjugated CCR8 targeting agent is administered second; (ii) the additional therapy is administered concurrently with the radioconjugated CCR8 targeting agent (e.g., the DDRi is administered orally once per day for n days, and the radioconjugated CCR8 targeting agent is administered intravenously in a single dose on one of days 2 through n−1 of the DDRi regimen); (iii) the additional therapy is administered concurrently with the radioconjugated CCR8 targeting agent (e.g., the DDRi is administered orally for a duration of greater than one month, such as orally once per day for 35 days, 42 days, 49 days, or a longer period during which the cancer being treated does not progress and during which the DDRi does not cause unacceptable toxicity, and the radioconjugated CCR8 targeting agent is administered intravenously in a single dose on a day within the first month of the DDRi regimen); and (iv) the radioconjugated CCR8 targeting agent is administered first (e.g., intravenously in a single dose or a plurality of doses over a period of weeks), and the additional therapy is administered second (e.g., the DDRi is administered orally once per day for 21 days, 28 days, 35 days, 42 days, 49 days, or a longer period during which the cancer being treated does not progress and during which the DDRi does not cause unacceptable toxicity). The radioconjugated CCR8 targeting agent and one or more additional therapeutic agents or therapies may, for example, be administered within 28 days of each other, within 21 days of each other, within 14 days of each other, within 7 days of each other, or within 6, 5, 4, 3, 2, or 1 days of each other (wherein a day is 24 hours). Additional permutations that would be obvious to one of skill in the art are also possible and within the scope of the presently claimed invention.

The radioconjugated CCR8 targeting agent(s) may, for example, be administered in combination with or in conjunction with an adoptive cell therapy (ACT), wherein the ACT may include administration of cells expressing a chimeric antigen receptor (CAR), or a T cell receptor (TCR), or may include tumor-infiltrating lymphocytes (TIL). As used herein, “ACT”, “adoptive cell therapy” and “adoptive cell transfer” may be used interchangeably. According to certain aspects, adoptive cell therapy (ACT) can refer to the transfer of cells to a patient with the goal of transferring the functionality and characteristics into the new host by engraftment of the cells.

As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing graft-versus-host disease issues. The adoptive transfer of autologous tumor infiltrating lymphocytes (TIL) or genetically redirected peripheral blood mononuclear cells has been used to successfully treat patients with advanced solid tumors, including melanoma and colorectal carcinoma, as well as patients with CD19-expressing hematologic malignancies. As described further herein, allogeneic cells can be edited to reduce alloreactivity and prevent graft-versus-host disease. Thus, use of allogeneic cells allows for cells to be obtained from healthy donors and prepared for use in patients as opposed to preparing autologous cells from a patient after diagnosis.

The population of cells expressing the CAR/TCR may, for example, include a population of activated T cells or natural killer cells (NK cells) or dendritic cells expressing the CAR/TCR which recognize an antigen. Dendritic cells are capable of antigen presentation, as well as direct killing of tumors. The population of cells expressing the CAR/TCR may, for example, include a population of gene-edited cells. Thus, the cells may be genetically modified to express the CAR or TCR.

As used herein, the term “gene-edited” CAR T cell or NK cell is synonymous with the terms “genetically engineered” CAR T cell or NK cell and “engineered” CAR T cell or NK cell. A gene-edited CAR T cell or NK cell that “fails to properly express” an antigen (e.g., checkpoint receptor such as PD1, Lag3 or TIM3) does not express the full-length, functional antigen. For example, a gene-edited CAR T cell that fails to properly express PD1 may fail to do so because, without limitation, (i) the cell's PD1 gene has been ablated, or (ii) the cell's PD1 gene has been otherwise altered so as not to yield a fully or even partially functional PD1 product. In other words, according to certain aspects, a gene-edited CAR T cell that fails to properly express PD1 may fail to do so because the cell's PD1 gene has been altered to diminish PD1 expression. Similarly, a gene-edited CAR T cell that “fails to properly express” a T cell receptor does not express the full-length, functional T cell receptor. Thus, the functional endogenous T cell receptor is replaced through engineering to “knock-out” a gene, such as by an alteration in the sequence of the gene that results in a decrease of function of the target gene, preferably such that target gene expression is undetectable or insignificant.

Alternatively, the engineering may be via “knock-in” of a target gene, such as by an alteration in a host cell genome that results in altered expression (e.g., increased, including ectopic) of the target gene, e.g., by introduction of an additional copy of the target gene or by operatively inserting a regulatory sequence that provides for enhanced expression of an endogenous copy of the target gene. For example, the functional endogenous T-cell receptor may be replaced through editing by a “knock-in” to the native TCR locus of an exogenously transduced CAR or recombinant TCR.

The gene-edited CAR T cells may include, without limitation, the following: (i) allogenic gene-edited CAR T cells that fail to properly express an antigen, e.g., PD1, but do properly express all other checkpoint receptors and T cell receptors; (ii) allogenic gene-edited CAR T cells that fail to properly express a particular T cell receptor but do properly express all checkpoint receptors and all other T cell receptors; and (iii) allogenic gene-edited CAR T cells that fail to properly express the antigen, e/g/, PD-1, and fail to properly express a particular T cell receptor, but do properly express all other checkpoint receptors and all other T cell receptors.

Examples of T cell gene editing to generate allogeneic, universal CAR T cells include the work of Eyquem and colleagues (Eyquem, 2017). In that study, the endogenous T cell receptor alpha constant locus (TRAC) was effectively replaced by a recombinant CAR gene construct. By this method, the recombinant CAR was placed effectively under the control of the cell's native TCR regulatory signals. By this same strategy, CARs or recombinant TCRs may be effectively inserted by knock-in into the T cell receptor beta constant gene locus (TRBC) or into the beta-2 microglobulin (B2) MHIC-I-related gene locus, known to be expressed in all T cells. Another example includes the work of Ren and colleagues (Ren, 2017). Recognizing that checkpoint receptors are immune-suppressive and may blunt the stimulation of exogenous autologous or allogeneic CAR T cells, this group exploited CRISPR/cas9 technology to ablate the endogenous TCR α and β loci (TRAC and TRBC) and the B2M gene, while also silencing the endogenous PD1 gene. With this approach, the engineered cells did not elicit graft-versus-host disease but did resist immune checkpoint receptor suppression.

Combination treatments of the present disclosure may combine a radioconjugated CCR8 targeting agent and one or more additional therapeutic agents or treatment modalities. Exemplary additional therapeutic agents include any disclosed herein, such as one or more immune checkpoint therapies (e.g., an antibody against an immune checkpoint inhibitor), one or more MICA blockades, one or more DDRi such as PARPi (e.g., antibody or small molecule that targets or inhibits PARP), one or more CD47 blockades, one or more radiosensitizers, an adoptive cell therapy, or any combination thereof. Moreover, combination treatment may also include a combination of more than one CCR8 targeting agent (e.g., CCR8 targeting agents that recognize or target different epitopes of CCR8), and optionally one or more additional therapeutic agents or modalities. Such combinations may result in synergistic effects in treating cancers.

An “article of manufacture” indicates a package containing materials useful for the treatment, prevention and/or diagnosis of the disorders described herein. The article of manufacture may include a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a radioconjugated CCR8 targeting agent according to aspects of the present disclosure.

A “label” or “package insert” is used to refer to instructions customarily included in commercial packages of therapeutic products that contain information about the indications, usage, dosage, administration, combination therapy, contraindications and/or warnings concerning the use of such therapeutic products. As used herein, a label may indicate that the composition is used for treating a CCR8-positive cancer and may optionally indicate administration routes and/or methods. Moreover, the article of manufacture may include (a) a first container with a composition contained therein, wherein the composition includes a radioconjugated CCR8 targeting agent; and (b) a second container with a composition contained therein, wherein the composition includes a further cytotoxic or otherwise therapeutic agent according to aspects of the present disclosure. Alternatively, or additionally, the article of manufacture may further include a second (or third) container including a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Unless otherwise defined, all technical and scientific terms used herein have the same meaning as commonly understood by one of ordinary skill in the art to which the present disclosure belongs. Although methods and materials similar or equivalent to those described herein can be used in the practice or testing described herein, suitable methods and materials are described below.

### ASPECTS OF THE INVENTION

The present disclosure provides methods and compositions for treating cancer by depleting tumor-infiltrating and/or tumor-resident Treg cells using radioconjugated CCR8 targeting agents. Suitable targeting agent that may be used include, without limitation, an antibody (fully human, humanized or chimeric IgG, or fragment thereof), an antibody mimetic, a peptide, or any other binding moiety that recognizes CCR8, which is then labeled with a radionuclide such as ²²Ac (a high energy alpha particle emitting radionuclide with a 10-day half-life and short path length [<100 μm]), ¹⁷⁷Lu, or ¹³¹I, or another radionuclide that delivers a therapeutic dose of radiation.

Accordingly, the disclosed radioconjugated CCR8 targeting agents can achieve therapeutic efficacy against all cancer types in which immunosuppressive Tregs hamper disease eradication. The mechanism of action for elimination of primary and metastatic tumors involves delivery of lethal radiation (from as low as a single radionuclide) to tumor-infiltrating CCR8-expressing Tregs and to adjacent malignant cells. Using this approach, both the pro-tumorigenic Tregs and the tumor itself are targeted. This radioimmunotherapy method is especially novel because ADCC, the mechanism of action relied on by current CCR8-targeting therapeutic candidates, is unable to cause direct collateral damage to tumor cells, whereas radioconjugated agents do so via crossfire effect.

Radioconjugated CCR8 targeting agents may also be clinically beneficial when combined with immunotherapy or adoptive cell transfer therapy. CCR8-targeted depletion of Tregs may lead to clinical efficacy in patients who have historically not responded to immunotherapies, such as inhibition of PD-(L)1 or CTLA-4 (Saleh, 2020; Hellman, 2018). In addition, due to calreticulin exposure on cancer cells that receive ionizing radiation through the crossfire effect, CD47 blockade would enhance macrophage-mediated phagocytosis of malignant cells, leading to improved antigen presentation to effector T cells and greater overall tumor response.

Accordingly, the radioconjugated CCR8 targeting agent(s) may be combined with or used in conjunction with one or more additional therapies to provide a potentially synergistic effect. Without limitation, additional therapies may include an immune checkpoint therapy or “blockade” (e.g., anti-PD(L)-1, anti-CTLA-4), a CD47 blockade, an anti-MICA antibody or MICA blockade, a DDRi, a radiosensitizer, externally delivered radiation, brachytherapy, and/or adoptive cell therapy (ACT).

When used in conjunction with adoptive cell transfer therapies, depletion of tumor infiltrating Treg cells by the radiolabeled CCR8 targeting agent may stimulate the tumoricidal activities of the adoptively transferred cells to generate more durable response in cancer patients. Treg depletion using a radioconjugated CCR8 targeting agent may also be beneficial in the context of adoptive cell transfer therapies because an immunosuppressive environment limits long-term response (Mardiana, 2019). Currently, clinical trials are ongoing that test the ability of PD-1 or CTLA-4 blockade to enhance the antitumor activity of adoptively transferred cells (e.g., CAR T cells) in various types of malignancies (Mardiana, 2019). However, it is known that not all patients derive long-term benefit from immune checkpoint inhibition (Maleki Vareki, 2017). Thus, additional means of stimulating antitumor immunity, such as through administration of the presently disclosed radioconjugated CCR8 targeting agents fills this unmet need among patient subpopulations by depletion of Tregs leading to enhanced effector T cell and/or NK cell function to enable tumor elimination.

In another combination approach, the presently disclosed radioconjugated CCR8 targeting agent may synergize with MICA-targeting biologics to enhance the activity of NK cells against malignant cells. MICA is a protein selectively expressed on the surface of many different cancer cell types and serves as a signal for cellular abnormality (e.g., oncogenic transformation, infection; Liu, 2019). NK cells recognize MICA through a cognate receptor and generate tumoricidal response (Liu, 2019). To therapeutically exploit NK cell-mediated cancer cell lysis, antibodies have been generated that recognize, stabilize, and increase cell surface expression of MICA for heightened tumor response (Xing, 2020). However, tumor-infiltrating Tregs are known to kill NK cells (Cao, 2007) and inhibit NK cell antitumor activity (Ogbomo, 2011; Smyth, 2006). By depleting Tregs using a radioconjugated CCR8 targeting agent—thereby increasing the number and activity of NK cells—the efficacy of MICA-targeting biologics may be intensified.

### CCR8 Targeting Agents

The present disclosure provides compositions and methods of use thereof of CCR8 targeting agents, such as anti-CCR8 antibodies, CCR8-binding antibody fragments, peptides, and small molecules. Several exemplary anti-CCR8 antibodies, as set forth below, are available and can be radiolabeled to provide a radioconjugated CCR8 targeting agent for use in or embodiment in the various aspects of the invention.

FPA175 developed by Five Prime Therapeutics (South San Francisco, CA, USA; Amgen) is an anti-CCR8 monoclonal antibody that has been engineered for enhanced ADCC activity (Rankin, 2020). When cell lines that express CCR8 were cocultured with NK cells in the presence of the antibody, higher level of cell death was observed compared to culturing in the presence of isotype control or non-ADCC-enhanced antibody (Rankin, 2020). In vivo efficacy was shown in murine tumor models using a different antibody (anti-mouse CCR8 monoclonal antibody; Rankin, 2020).

HBM1022 (Harbour BioMed, Hong Kong, China and Natick, MA, USA) is another monoclonal antibody that targets CCR8. In preclinical syngeneic and humanized models, HBM1022 synergized with anti-PD1 immunotherapy to elicit antitumor response (Lu, 2020). HBM1022 and/or any of the anti-CCR8 antibodies disclosed in Int'l Pub. No. WO2022042690 may be radiolabeled for use or embodiment in any of the various aspects of the present invention.

SRF 114 (Surface Oncology, Cambridge, MA, USA) is an antibody that recognizes CCR8 and was discovered using Vaccinia Virus ActivMab™ technology (Lake, 2020). Displaying human-specific reactivity, SRF 114 caused NK cell-mediated target cell destruction in CCR8 expressing cells and resulted in depletion of Tregs isolated from primary tumor resections (Lake, 2020). SRF114 and/or any of the anti-CCR8 antibodies disclosed in U.S. Pub. No. 20210238292 may be radiolabeled for use in or embodiment in any of the various aspects of the present invention.

Exemplary anti-CCR8 antibodies or CCR8-binding antibody fragments that specifically bind the N-terminal extracellular domain of human CCR8, and which may be radiolabeled for use in or embodiment in the various aspects of the invention may include:
(a) a CDR-H1 including the amino acid sequence set forth in SEQ ID NO:45, a CDR-H2 including the amino acid sequence set forth in SEQ ID NO:46, a CDR-H3 including the amino acid sequence set forth in SEQ ID NO:47, a CDR-L1 including the amino acid sequence set forth in SEQ ID NO:48, a CDR-L2 including the amino acid sequence set forth in SEQ ID NO:49, and a CDR-L3 including the amino acid sequence set forth in SEQ ID NO:50;
(b) a CDR-H1 including the amino acid sequence set forth in SEQ ID NO: 105, a CDR-H2 including the amino acid sequence set forth in SEQ ID NO:106, a CDR-H3 including the amino acid sequence set forth in SEQ ID NO:107, a CDR-L1 including the amino acid sequence set forth in SEQ ID NO:108, a CDR-L2 including the amino acid sequence set forth in SEQ ID NO:109, and a CDR-L3 including the amino acid sequence set forth in SEQ ID NO:110;
(c) a CDR-H1 including the amino acid sequence set forth in SEQ ID NO: 115, a CDR-H2 including the amino acid sequence set forth in SEQ ID NO:116, a CDR-H3 including the amino acid sequence set forth in SEQ ID NO:117, a CDR-L1 including the amino acid sequence set forth in SEQ ID NO:118, a CDR-L2 including the amino acid sequence set forth in SEQ ID NO:119, and a CDR-L3 including the amino acid sequence set forth in SEQ ID NO:120;
(d) a CDR-H1 including the amino acid sequence set forth in SEQ ID NO: 135, a CDR-H2 including the amino acid sequence set forth in SEQ ID NO:136, a CDR-H3 including the amino acid sequence set forth in SEQ ID NO:137, a CDR-L1 including the amino acid sequence set forth in SEQ ID NO:138, a CDR-L2 including the amino acid sequence set forth in SEQ ID NO:139, and a CDR-L3 including the amino acid sequence set forth in SEQ ID NO:140;
(e) a CDR-H1 including the amino acid sequence set forth in SEQ ID NO:145, a CDR-H2 including the amino acid sequence set forth in SEQ ID NO:146, a CDR-H3 including the amino acid sequence set forth in SEQ ID NO:147, a CDR-L1 including the amino acid sequence set forth in SEQ ID NO:148, a CDR-L2 including the amino acid sequence set forth in SEQ ID NO:149, and a CDR-L3 including the amino acid sequence set forth in SEQ ID NO: 150;
(f) a CDR-H1 including the amino acid sequence set forth in SEQ ID NO:5, a CDR-H2 including the amino acid sequence set forth in SEQ ID NO:6, a CDR-H3 including the amino acid sequence set forth in SEQ ID NO:7, a CDR-L1 including the amino acid sequence set forth in SEQ ID NO:8, a CDR-L2 including the amino acid sequence set forth in SEQ ID NO:9, and a CDR-L3 including the amino acid sequence set forth in SEQ ID NO:10;
(g) a CDR-H1 including the amino acid sequence set forth in SEQ ID NO: 15, a CDR-H2 including the amino acid sequence set forth in SEQ ID NO:16, a CDR-H3 including the amino acid sequence set forth in SEQ ID NO:17, a CDR-L1 including the amino acid sequence set forth in SEQ ID NO:18, a CDR-L2 including the amino acid sequence set forth in SEQ ID NO:19, and a CDR-L3 including the amino acid sequence set forth in SEQ ID NO:20;
(h) a CDR-H1 including the amino acid sequence set forth in SEQ ID NO:25, a CDR-H2 including the amino acid sequence set forth in SEQ ID NO:26, a CDR-H3 including the amino acid sequence set forth in SEQ ID NO:27, a CDR-L1 including the amino acid sequence set forth in SEQ ID NO:28, a CDR-L2 including the amino acid sequence set forth in SEQ ID NO:29, and a CDR-L3 including the amino acid sequence set forth in SEQ ID NO:30;
(i) a CDR-H1 including the amino acid sequence set forth in SEQ ID NO:35, a CDR-H2 including the amino acid sequence set forth in SEQ ID NO:36, a CDR-H3 including the amino acid sequence set forth in SEQ ID NO:37, a CDR-L1 including the amino acid sequence set forth in SEQ ID NO:38, a CDR-L2 including the amino acid sequence set forth in SEQ ID NO:39, and a CDR-L3 including the amino acid sequence set forth in SEQ ID NO:40;
(i) a CDR-H1 including the amino acid sequence set forth in SEQ ID NO:55, a CDR-H2 including the amino acid sequence set forth in SEQ ID NO:56, a CDR-H3 including the amino acid sequence set forth in SEQ ID NO:57, a CDR-L1 including the amino acid sequence set forth in SEQ ID NO:58, a CDR-L2 including the amino acid sequence set forth in SEQ ID NO:59, and a CDR-L3 including the amino acid sequence set forth in SEQ ID NO:60;
(k) a CDR-H1 including the amino acid sequence set forth in SEQ ID NO:65, a CDR-H2 including the amino acid sequence set forth in SEQ ID NO:66, a CDR-H3 including the amino acid sequence set forth in SEQ ID NO:67, a CDR-L1 including the amino acid sequence set forth in SEQ ID NO:68, a CDR-L2 including the amino acid sequence set forth in SEQ ID NO:69, and a CDR-L3 including the amino acid sequence set forth in SEQ ID NO:70;
(l) a CDR-H1 including the amino acid sequence set forth in SEQ ID NO:75, a CDR-H2 including the amino acid sequence set forth in SEQ ID NO:76, a CDR-H3 including the amino acid sequence set forth in SEQ ID NO:77, a CDR-L1 including the amino acid sequence set forth in SEQ ID NO:78, a CDR-L2 including the amino acid sequence set forth in SEQ ID NO:79, and a CDR-L3 including the amino acid sequence set forth in SEQ ID NO:80;
(m) a CDR-H1 including the amino acid sequence set forth in SEQ ID NO:85, a CDR-H2 including the amino acid sequence set forth in SEQ ID NO:86, a CDR-H3 including the amino acid sequence set forth in SEQ ID NO:87, a CDR-L1 including the amino acid sequence set forth in SEQ ID NO:88, a CDR-L2 including the amino acid sequence set forth in SEQ ID NO:89, and a CDR-L3 including the amino acid sequence set forth in SEQ ID NO:90;
(n) a CDR-H1 including the amino acid sequence set forth in SEQ ID NO:95, a CDR-H2 including the amino acid sequence set forth in SEQ ID NO:96, a CDR-H3 including the amino acid sequence set forth in SEQ ID NO:97, a CDR-L1 including the amino acid sequence set forth in SEQ ID NO:98, a CDR-L2 including the amino acid sequence set forth in SEQ ID NO:99, and a CDR-L3 including the amino acid sequence set forth in SEQ ID NO:100;
(o) a CDR-H1 including the amino acid sequence set forth in SEQ ID NO: 125, a CDR-H2 including the amino acid sequence set forth in SEQ ID NO:126, a CDR-H3 including the amino acid sequence set forth in SEQ ID NO:127, a CDR-L1 including the amino acid sequence set forth in SEQ ID NO:128, a CDR-L2 including the amino acid sequence set forth in SEQ ID NO:129, and a CDR-L3 including the amino acid sequence set forth in SEQ ID NO:130;
(p) a CDR-H1 including the amino acid sequence set forth in SEQ ID NO: 155, a CDR-H2 including the amino acid sequence set forth in SEQ ID NO:156, a CDR-H3 including the amino acid sequence set forth in SEQ ID NO:157, a CDR-L1 including the amino acid sequence set forth in SEQ ID NO:158, a CDR-L2 including the amino acid sequence set forth in SEQ ID NO:159, and a CDR-L3 including the amino acid sequence set forth in SEQ ID NO:160; or
(q) a CDR-H1 including the amino acid sequence set forth in SEQ ID NO: 165, a CDR-H2 including the amino acid sequence set forth in SEQ ID NO:166, a CDR-H3 including the amino acid sequence set forth in SEQ ID NO:167, a CDR-L1 including the amino acid sequence set forth in SEQ ID NO:168, a CDR-L2 including the amino acid sequence set forth in SEQ ID NO:169, and a CDR-L3 including the amino acid sequence set forth in SEQ ID NO:170.

BMS-986340 (Bristol Myers Squibb, New York, NY, USA) was also demonstrated to deplete Tregs by binding to CCR8, and in murine tumor models, this antibody as a single agent led to pro-inflammatory responses concomitant with tumor reduction. When combined with anti-PD-1 therapy, BMS-986340 resulted in even greater response (Lan, 2020). BMS-986340 and/or any of the anti-CCR8 antibodies disclosed in Int'l Pub. No. WO2021194942 may be radiolabeled for use or embodiment in any of the various aspects of the present invention.

JTX-1811 (Jounce Therapeutics, Cambridge, MA, USA), an anti-CCR8 antibody being co-developed with Gilead, demonstrated robust ADCC against cells that expressed CCR8 at a level comparable to that of tumor infiltrating Tregs but not against cells with lower expression (Depis, 2020). Furthermore, the investigators demonstrated preclinical efficacy with anti-PD-1-resistant tumor models and showed synergy with anti-PD-1 therapy when combined (Depis, 2020). JTX-1811 and/or any of the anti-CCR8 antibodies or fusion proteins disclosed in U.S. Pub. No. 20210277129 or Int'l Pub. No. WO2021163064 may be radiolabeled for use or embodiment in any of the various aspects of the present invention.

Another monoclonal antibody (unnamed at the present time) against CCR8 is being developed by Shionogi to reduce tumor burden (Kawashima, 2020).

In contrast to full-length IgG molecules, nanobodies against CCR8 have also been developed by Oncurious. Experiments in mice showed that tetravalent nanobody-Fc fusion construct caused ADCC-mediated Treg depletion and tumor suppression. Importantly, the nanobody-Fc fusion construct specifically depleted tumor infiltrating Tregs while having no discernible effect on peripheral Tregs. This study also demonstrated synergistic effect with anti-PD-1 therapy and the establishment of immunological memory that led to tumor rejection when re-challenged (Van Damme, 2021). Any of the anti-CCR8 nanobodies, single domain antibodies and other CCR8 binders disclosed in Int'l Pub. No. WO2022003156 may be radiolabeled for use or embodiment in any of the various aspects of the present invention.

Antibody clone SA214G2 (rat IgG2b) recognizes mouse CCR8. This antibody reduced tumor growth and prolonged survival in a murine model of colorectal cancer (Villareal, 2018).

The complete amino acid sequence for human CCR8 (UniProt P51685) is provided in SEQ ID NO: 171. The amino acid sequence of the N-terminal extracellular domain of human CCR8 is specifically provided in SEQ ID NO: 172. Still further antibodies against the extracellular domain of human CCR8, which may be used in or embodied in the various aspects of the invention, may be generated using well established methods known in the art.

Wherever in this disclosure antibodies, such as anti-human-CCR8 antibodies, are described for use in or embodiment in the various aspects of the invention, it should be understood that also provided are aspects using or embodying antibodies that include one, two or three of the heavy chain CDRs (CDR-H₁₋₃) or the heavy chain variable region (with or without any signal sequence) of said antibodies, and/or one, two or three of the light chain CDRs (CDR-L₁₋₃) or the light chain variable region (with or without any signal sequence) of said antibodies. Further, wherever in this disclosure antibody light chains or the variable region thereof or antibody heavy chains or the variable region thereof are described with N-terminal signal sequences, it should be understood that corresponding versions without the signal sequences are also disclosed and may be used in or embodied in the various aspects of the invention. CDRs may, for example, be delineated according to the Kabat or IMGT numbering conventions.

Radio-conjugation is an approach to augment the therapeutic profile of a targeting agent. By arming a biologic with a radionuclide that causes lethal DNA damage to the targeted cell, antibody radio-conjugates (ARCs) can lead to potent eradication of any cell type of interest, provided there are specific surface markers that enable a therapeutic window of opportunity for targeted deposition of the toxic payload. Because CCR8 is highly expressed only in tumor infiltrating Tregs (De Simone, 2016; Plitas, 2016), the ARC approach would be wholly amenable to targeted depletion of these cells. Radionuclides are unique from the other conjugation approaches because internalization of the biologic-conjugate complex is not necessary; lethal radiation can be delivered from the cell surface. Of radionuclides that are in preclinical and clinical trials, ¹⁷⁷Lu [Lutetium], ²¹³Bi [Bismuth], ¹³¹I [Iodine]), and ²²⁵Ac [Actinium] exhibit a favorable profile for conjugation to biologics that target tumors (Nelson, 2020).

²²⁵Ac is a radionuclide that emits alpha particles with high linear energy transfer (80 keV/μm) over a short distance (50-100 μm). The clusters of double strand DNA breaks that result after exposure to alpha particles are much more difficult to repair than damage from radionuclides that emit beta particles with low linear energy transfer (0.2 keV/μm). The inability to repair DNA damage eventually leads to cell death. This potency of alpha particles can be exploited for targeted radioimmunotherapy, whereby ²²⁵Ac is conjugated to an antibody via a chelator. In this way, lethal radiation can be delivered specifically to cells bearing the antibody target (e.g., CCR8), allowing precise ablation of tumor infiltrating Tregs while minimizing damage to healthy tissues.

Furthermore, the long half-life of ²²⁵Ac (10 days) makes this radionuclide particularly attractive for therapeutic evaluation. ²²⁵Ac can be conjugated to any biologic (e.g., full-length antibody, scFv, Fab, Fab₂, peptide) via a linker-chelator moiety, and in preclinical and clinical studies, dodecane tetraacetic acid (DOTA) is commonly used to stably chelate ²²⁵Ac, although other chelating agents may be used. Examples of ²²⁵Ac-conjugated anti-cancer agents include: CD33-targeting ²²⁵Ac-lintuzumab to treat acute myeloid leukemia, glypican-3-targeting ²²⁵Ac-GC33 to treat hepatocellular carcinoma, CD45-targeting ²²⁵Ac-BC8 to treat multiple myeloma, and PSMA-targeting ²²⁵Ac-PSMA617 to treat prostate cancer (Nelson, 2020; Garg, 2021; Bell, 2020).

CD33 targeting agents may also be used to deplete myeloid-derived suppressor cells (MDSCs) found in solid tumors. Thus, according to one aspect of the invention, one or more radiolabeled CCR8 targeting agents may be used in combination or conjunction with a CD33 targeting agent such as a radiolabeled CD33 targeting agent (e.g., radiolabeled with any of the radionuclides described herein), such as a radiolabeled anti-CD33 monoclonal antibody or a radiolabeled CD33-binding antibody fragment, such as an ²²⁵Ac labeled anti-CD33 antibody, such as ²²⁵Ac lintuzumab, or a cytotoxin conjugated CD33 targeting agent, such as a CD33-targeted ADC, such as gemtuzumab ozogamicin, to deplete both suppressive tumor Treg cells and myeloid derived suppressor cells (MDSCs), for example, for the treatment of a solid tumor, such as any of those disclosed herein, in a mammalian subject such as a human patient. Suitable CD33 targeting agents, which may be radiolabeled or drug conjugated, include, for example, lintuzumab, gemtuzumab, vadastuximab, antibodies having the heavy chain and light chain CDRs of the preceding monoclonal antibodies, and CD33-binding fragments of any of the preceding monoclonal antibodies. The CCR8 and CD33 targeting agents may, for example be administered simultaneously (as one composition or in separate compositions), in a temporally overlapping manner, or sequentially with one before the other.

Accordingly, therapeutic methods for treating tumors including CCR8-positive Tregs are provided. The methods may, for example, also include diagnostic steps to determine if and/or to what extent the patient has a CCR8-positive tumor (e.g., tumor having CCR8 positive Tregs infiltrated therein), such as by identifying CCR8 positive cells within solid tumors.

The therapeutic methods may, for example, include administration of a radiolabeled CCR8 targeting agent, such as a radiolabeled antibody, peptide, or small molecule that targets CCR8, either alone or in combination with an additional therapeutic agent or modality. The additional agent or modality may, for example, be any one or more of administration of an immune checkpoint therapy, a DDRi, a CD47 blockade, a MICA blockade, a chemotherapeutic agent, or radiation therapy (i.e., external beam radiation or brachytherapy), or an adoptive cell therapy, or a radiolabeled, drug-conjugated, or unlabeled targeting agent, such as an antibody, directed against a target antigen other than CCR8.

The CCR8 targeting agent may, for example, be administered to the patient in a patient-specific composition in one or more doses.

The patient may, for example, be monitored at intervals during the therapy for the presence of CCR8 positive cells to evaluate the reduction in CCR8-positive cells. Detecting a decreased number of the CCR8-positive cells after treatment with the CCR8 targeting agent, as compared to the number of CCR8-positive cells prior to treatment may indicate effectiveness of the CCR8 targeting agent in treating a CCR8-positive cancer in the mammalian subject.

The method of treating cancer may, for example, include identifying a patient that has a CCR8-positive cancer by identifying CCR8-positive cells and administering to the patient an effective amount of a radiolabeled CCR8 targeting agent, either alone or in combination or conjunction with an additional therapeutic agent treatment or treatment modality. The additional agent or treatment modality may, for example, be any one or more of administration of an immune checkpoint therapy, a DDRi, a CD47 blockade, a MICA blockade, a chemotherapeutic agent, or radiation therapy (e.g., external beam radiation or brachytherapy), an adoptive cell therapy, or a radiolabeled, drug-conjugated, or unlabeled targeting agent, such as an antibody, directed against a target antigen other than CCR8. The chemotherapeutic agent may, for example, be a radiosensitizer.

The radioconjugated CCR8 targeting agent may, for example, be administered to a patient that has also already undergone a previous treatment, such as surgery for treatment of the cancer, such as to remove all or a portion of a solid tumor.

### CCR8 Targeting Agent Multi-Specificity and Multi-Antigen Targeting Aspects

Objects of the present disclosure include providing a radioconjugated CCR8 targeting agent that is useful in diagnostic assays or imaging and/or effective in therapeutic interventions for proliferative disorders, such as for the treatment of solid tumors. Mechanisms by which the presently disclosed radioconjugated CCR8 targeting agents can effect a therapeutic response include targeted DNA damage caused by the ionizing radiation emitted by the radiolabel.

Thus, in one aspect, the present disclosure provides for treatment of proliferative diseases or disorders, such as solid tumors, with a radioconjugated CCR8 targeting agent that functions to target ionizing radiation to cells expressing CCR8, i.e., Tregs in the tumor microenvironment, thereby inducing DNA damage in those cells. Exemplary CCR8 targeting agents that may be used include anti-CCR8 antibodies and CCR8-binding antibody fragments, such as Fab, Fab₂ and scFv molecules, that bind to an epitope of CCR8.

The present disclosure further provides methods of treating a proliferative disease or disorder, such as any of those disclosed herein, in a mammal such as a human patient, which include administration of a radiolabeled multi-specific targeting agent, such as a multi-specific antibody, against two or more epitopes of CCR8, or against an epitope of CCR8 and against one or more additional different antigens.

The present disclosure still further provides methods of treating a proliferative disease or disorder, such as any of the cancers disclosed herein, in a mammal such as a human patient, which include administration of a radiolabeled CCR8 targeting agent (such as an antibody), which may be monospecific or multi-specific, and one or more separate targeting agents (such as antibodies) against one or more antigens different than CCR8, which one or more separate targeting agents are therapeutically active and may themselves be radiolabeled with the same or different radiolabels as the CCR8 targeting agent, and/or may be drug conjugated, or may be unconjugated/unlabeled but therapeutically active. Thus, in one aspect the present disclosure also provides methods of treating a proliferative disease or disorder which includes administration of a first radiolabeled antibody against at least one antigen of CCR8, and administration of a second antibody which may also be radiolabeled, wherein the second antibody is against a different epitope of CCR8 than the first antibody and/or or is against an epitope of a different antigen, such as a different cancer-associated antigen or different suppressor immune cell associated antigen. Specific different (non-CCR8) antigens that may be targeted and exemplary targeting agents for these different antigens which may be used in or embodied in the various aspects of the invention, for example, in radiolabeled, drug conjugated, or unconjugated forms are further described hereinbelow.

The additional different antigens may be antigens differentially expressed on cells involved in various proliferative diseases or disorders, such as on cancer cells themselves and/or on other cells involved in solid tumors such as suppressive immune cells. For example, the additional different antigens may be selected from the group including mammalian, for example human, forms of CD33, DR5, 5T4, HER2 (ERBB2; Her2/neu), HER3, TROP2, mesothelin, TSHR, CD19, CD123, CD22, CD30, CD45, CD171, CD138, CS-1, CLL-1, GD2, GD3, B-cell maturation antigen (BCMA), T antigen (T Ag), Tn Antigen (Tn Ag), prostate specific membrane antigen (PSMA), ROR1, FLT3, fibroblast activation protein (FAP), a Somatostatin receptor, Somatostatin Receptor 2 (SSTR2), Somatostatin Receptor 5 (SSTR5), gastrin-releasing peptide receptor (GRPR), NKG2D ligands (such as MICA, MICB, RAET1E/ULBP4, RAET1G/ULBP5, RAET1H/ULBP2, RAET1/ULBP1, RAET1L/ULBP6, and RAET1N/ULBP3), tenascin, tenascin-C, CEACAM5, Cadherin-3, CCK2R, Neurotensin receptor type 1 (NTSR1), human Kallikrein 2 (hK2), norepinephrine transporter, Integrin alpha-V-beta-6, CD37, CD66, CXCR4, Fibronectin extradomain B (EBD), LAT-1, Carbonic anhydrase IX (CAIX), B7-H3 (a/k/a CD276), Disialoganglioside GD2 Antigen (GD2), calreticulin, phosphatidylserine, GRP78 (BiP), TAG72, CD38, CD44v6, CEA, EPCAM, B7H3, KIT, IL-13Ra2, interleukin-11 receptor a (IL-1 1Ra), PSCA, PRSS21, VEGFR2, LewisY, CD24, platelet-derived growth factor receptor-beta (PDGFR-beta), SSEA-4, CD20, Folate receptor alpha (FRa), LYPD3 (C4.4A), MUC1, epidermal growth factor receptor (EGFR), EGFRvIII, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD 179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-la, MAGE-A1, legumain, HPV E6,E7, MAGE A1, MAGEA3, MAGEA3/A6, ETV6-AML, sperm protein 17, XAGE1, Tie 2, MAD-CT-1, MAD-CT-2, Fos-related antigen 1, prostein, survivin and telomerase, PCTA-1/Galectin 8, KRAS, MelanA/MARTI, Ras mutant, hTERT, sarcoma translocation breakpoints, ML-IAP, ERG (TMPRSS2 ETS fusion gene), NA17, PAX3, Androgen receptor, Cyclin B 1, MYCN, RhoC, TRP-2, CYP1B 1, BORIS, SART3, PAX5, OY-TES 1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LTLRA2, CD300LF, CLEC12A, BST2, EMR2, LY75, GPC3, FCRL5, GPA7, and IGLL1.

Exemplary DR5 (death receptor 5) targeting agents that may be radiolabeled, unlabeled or drug-conjugated for use in the invention include the monoclonal anti-DR5 antibodies mapatumumab, conatumumab, lexatumumab, tigatuzumab, drozitumab, and LBY-135. Such DR5 targeting agents may, for example, be used in combination with a radiolabeled CCR8 targeting agent for the treatment of ovarian, breast, cervical prostate, gastric, bladder, lung, melanoma, colorectal and squamous cell carcinoma cancers and any of the cancers disclosed herein.

Exemplary 5T4 (Trophoblast glycoprotein (TBPG)) targeting agents that may be radiolabeled, drug-conjugated, or unlabeled for use in the invention include the anti-5T4 monoclonal antibodies MED10641, ALG.APV-527, Tb535, H6-DM5, and ZV0508, as well as 5T4Fab-SEA/E-120 (SEQ ID NO:312), Naptumomab estafenatox (reported as SEQ ID NO:313 (chimeric heavy chain component) non-covalently bound to SEQ ID NO:314 (light chain component)) or the Fab portion thereof only, and an anti-5TF Fab molecule including a heavy chain component corresponding to residues 1 to 222 of SEQ ID NO:312 and a light chain component residues 459 to 672 of SEQ ID NO:312. Such 5T4 targeting agents may, for example, be used in combination with a radiolabeled CCR8 targeting agent for the treatment of ovarian, head and neck, breast, prostate, gastric, bladder, lung, melanoma, colorectal and squamous cell carcinoma cancers and any of the cancers disclosed herein.

Exemplary HER2 (ERBB2) targeting agents that may be radiolabeled, drug-conjugated, or unlabeled for use in the invention include the monoclonal antibodies trastuzumab and pertuzumab. Applicants have successfully conjugated Trastuzumab with p-SCN-DOTA and radiolabeled the composition with ²²⁵Ac or ¹⁷⁷Lu. Exemplary ADCs targeting HER2 that may be used include fam-trastuzumab deruxtecan-nxki (Enhertu®; AstraZeneca/Daiichi Sankyo) and Trastuzumab emtansine (Roche/Genentech). The anti-HER2 antibody may, for example, also be a multi-specific antibody, such as bispecific antibody, against any available epitope of HER3/HER2 such as MM-111 and MM-141/Istiratumab from Merrimack Pharmaceuticals, MCLA-128 from Merus NV, and MEHD7945A/Duligotumab from Genentech. HER2 targeting agents may, for example, be used in combination with a radiolabeled CCR8 targeting agent in the treatment of HER2-expressing cancers such as ovarian, breast, metastatic breast, esophageal, lung, cervical, and endometrial cancers including but not limited to those that are both HER2- and HER3-positive.

The amino acid sequences of the heavy chain and the light chain of Trastuzumab reported by DrugBank Online are: heavy chain (SEQ ID NO:280) and light chain (SEQ ID NO:281) and a HER2 binding antibody including one or both of said chains may be embodied in or used in the various embodiments of the invention.

The amino acid sequences of the heavy chain and the light chain of Pertuzumab reported by DrugBank Online are: heavy chain (SEQ ID NO:282) and light chain (SEQ ID NO:283) and a HER2 binding antibody including one or both of said chains may be embodied in or used in the various embodiments of the invention.

An exemplary HER3 antibody that may be radiolabeled and embodied in and/or used in the presently disclosed invention may, for example, include a murine monoclonal antibody against HER3 including a heavy chain having the amino acid sequence as set forth in SEQ ID NO:187 or 189 and/or a light chain having the amino acid sequence as set forth in SEQ ID NO:188 or 190, or an antibody such as a humanized antibody derived from one or more of said sequences. An exemplary HER3 antibody that may be radiolabeled and embodied in and/or used in the presently disclosed invention may include or a heavy chain with an N-terminal region having the sequence set forth in SEQ ID NO:191 and/or a light chain with an N-terminal region having the sequence as set forth in SEQ ID NO:192. A HER3 antibody that may be similarly embodied or used in various aspect of the invention may, for example, include the heavy chain variable region having the amino acid sequence as set forth in SEQ ID NO:185, and/or a light chain variable region having an amino acid sequence as set forth in SEQ ID NO:186; and/or a heavy chain including one or more of CDR1, CDR2 and CDR3 having the amino acid sequences respectively set forth in SEQ ID NOS:179-181, and/or a light chain with one or more of the CDR1, CD2 and CDR3 having the amino acid sequences respectively set forth in SEQ ID NOS:182-184. A HER3 antibody embodied in and/or used in any of the aspects of the invention may, for example, include any combination of the aforementioned light chain sequences and/or heavy chain sequences.

An exemplary HER3 antibody includes an immunoglobulin heavy chain variable region including a CDR-H1 including SEQ ID NO:193, a CDR-H2 including SEQ ID NO:194, and a CDR-H3 including SEQ ID NO:195, and/or an immunoglobulin light chain variable region including a CDR-L1 including SEQ ID NO:196, a CDR-L2 including SEQ ID NO:197, and a CDR-L3 including SEQ ID NO: 198. An exemplary An exemplary HER3 antibody includes an immunoglobulin heavy chain variable region including SEQ ID NO:199 and/or an immunoglobulin light chain variable region including SEQ ID NO:200. An exemplary HER3 antibody includes an immunoglobulin heavy chain amino acid sequence of SEQ ID NO:201 and/or an immunoglobulin light chain amino acid sequence of SEQ ID NO:202.

An exemplary HER3 antibody includes an immunoglobulin heavy chain variable region including a CDR-H1 including SEQ ID NO:203, a CDR-H2 including SEQ ID NO:204, and a CDR-H3 including SEQ ID NO:205; and/or an immunoglobulin light chain variable region including a CDR-L1 including SEQ ID NO:206, a CDR-L2 including SEQ ID NO:207, and a CDR-L3 including SEQ ID NO:208. An exemplary HER3 antibody includes an immunoglobulin heavy chain variable region including SEQ ID NO:209 and/or an immunoglobulin light chain variable region including SEQ ID NO:210. An exemplary HER3 antibody includes an immunoglobulin heavy chain amino acid sequence of SEQ ID NO:211 and/or an immunoglobulin light chain amino acid sequence of SEQ ID NO:212.

An exemplary HER3 antibody includes an immunoglobulin heavy chain variable region including a CDR-H1 including SEQ ID NO:213, a CDR-H2 including SEQ ID NO:214, and a CDR-H3 including SEQ ID NO:215; and/or an immunoglobulin light chain variable region including a CDR-L1 including SEQ ID NO:216, a CDR-L2 including SEQ ID NO:217, and a CDR-L3 including SEQ ID NO:218. An exemplary HER3 antibody includes an immunoglobulin heavy chain variable region including SEQ ID NO:219, and/or an immunoglobulin light chain variable region SEQ ID NO:220. An exemplary HER3 antibody includes an immunoglobulin heavy chain amino acid sequence of SEQ ID NO:221 and an immunoglobulin light chain amino acid sequence of SEQ ID NO:222.

An exemplary HER3 antibody includes an immunoglobulin heavy chain variable region including a CDR-H1 including SEQ ID NO:223, a CDR-H2 including SEQ ID NO:224, and a CDR-H3 including SEQ ID NO:225; and/or an immunoglobulin light chain variable region including a CDR-L1 including SEQ ID NO:226, a CDR-L2 including SEQ ID NO:207, and a CDR-L3 including SEQ ID NO:227. An exemplary HER3 antibody includes an immunoglobulin heavy chain variable region including SEQ ID NO:228 and/or an immunoglobulin light chain variable region including SEQ ID NO:229. An exemplary HER3 antibody includes an immunoglobulin heavy chain amino acid sequence of SEQ ID NO:230 and/or an immunoglobulin light chain amino acid sequence of SEQ ID NO:231.

An exemplary HER3 antibody includes an immunoglobulin heavy chain variable region including a CDR-H1 including SEQ ID NO:232, a CDR-H2 including SEQ ID NO:233, and a CDR-H3 including SEQ ID NO:234; and/or an immunoglobulin light chain variable region including a CDR-L1 including SEQ ID NO:206, a CDR-L2 including SEQ ID NO:207, and a CDR-L3 including SEQ ID NO:208. An exemplary HER3 antibody includes an immunoglobulin heavy chain variable region including SEQ ID NO:235 and/or an immunoglobulin light chain variable region including SEQ ID NO:236. An exemplary HER3 antibody includes an immunoglobulin heavy chain amino acid sequence of SEQ ID NO:237 and/or an immunoglobulin light chain amino acid sequence of SEQ ID NO:238.

An exemplary HER3 antibody includes an immunoglobulin heavy chain variable region including a CDR-H1 including SEQ ID NO:239, a CDR-H2 including SEQ ID NO:240, and a CDR-H3 including SEQ ID NO:241; and/or an immunoglobulin light chain variable region including a CDR-L1 including SEQ ID NO:242, a CDR-L2 including SEQ ID NO:243, and a CDR-L3 including SEQ ID NO:244. An exemplary HER3 antibody includes an immunoglobulin heavy chain variable region including SEQ ID NO:245, and/or an immunoglobulin light chain variable region including SEQ ID NO:246. An exemplary HER3 antibody includes an immunoglobulin heavy chain amino acid sequence of SEQ ID NO:247 and an immunoglobulin light chain amino acid sequence of SEQ ID NO:248.

An exemplary HER3 antibody includes an immunoglobulin heavy chain variable region including a CDR-H1 including SEQ ID NO:249, a CDR-H2 including SEQ ID NO:250, and a CDR-H3 including SEQ ID NO:244; and/or an immunoglobulin light chain variable region including a CDR-L1 including SEQ ID NO:206, a CDR-L2 including SEQ ID NO:207, and a CDR-L3 including SEQ ID NO:208. An exemplary HER3 antibody includes an immunoglobulin heavy chain variable region including SEQ ID NO:251, and/or an immunoglobulin light chain variable region including SEQ ID NO:252. An exemplary HER3 antibody includes an immunoglobulin heavy chain amino acid sequence of SEQ ID NO:253 and/or an immunoglobulin light chain amino acid sequence of SEQ ID NO:254.

An exemplary HER3 antibody includes an immunoglobulin heavy chain amino acid sequence of SEQ ID NO:255 and/or an immunoglobulin light chain amino acid sequence of SEQ ID NO:256.

An exemplary HER3 antibody includes an immunoglobulin light chain variable region including SEQ ID NOS:264, 265, 266, 267, 268 or 269 and/or a heavy chain variable region including SEQ ID NOS:257, 258, 259, 260, 261, 262 or 263.

An exemplary HER3 antibody includes an immunoglobulin heavy chain sequence including SEQ ID NO:270, 272, 273, 276 or 277 and/or an immunoglobulin light chain sequence including SEQ ID NO:271, 274, 275, 278 or 279.

Exemplary HER3 antibodies also include Barecetamab (ISU104) from Isu Abxis Co and any of the HER3 antibodies disclosed in U.S. Pat. No. 10,413,607.

Exemplary HER3 antibodies also include HMBD-001 (10D1F) from Hummingbird Bioscience Pte. and any of the HER3 antibodies disclosed in International Pub. Nos. WO 2019185164 and WO2019185878, U.S. Pat. No. 10,662,241; and U.S. Pub. Nos. 20190300624, 20210024651, and 20200308275.

Exemplary HER3 antibodies also include the HER2/HER3 bispecific antibody MCLA-128 (i.e., Zenocutuzumab) from Merus N.V.; and any of the HER3 antibodies, whether monospecific or multi-specific, disclosed in U.S. Pub. Nos. 20210206875, 20210155698, 20200102393, 20170058035, and 20170037145.

Exemplary HER3 antibodies also include the HER3 antibody Patritumab (U3-1287), an antibody including heavy chain sequence SEQ ID NO:106 and/or light chain sequence SEQ ID NO:285 which are reported chains of Patritumab, and any of the HER3 antibodies disclosed in U.S. Pat. Nos. 9,249,230 and 7,705,130 and International Pub. No. WO2007077028.

Exemplary HER3 antibodies also include the HER3 antibody MM-121 and any of the HER3 antibodies disclosed in U.S. Pat. No. 7,846,440 and Int'l Pub. No. WO2008100624.

Exemplary HER3 antibodies also include the EGFR/HER3 bispecific antibody DL1 and any of the HER3 antibodies, whether monospecific or multi-specific, disclosed in U.S. Pat. Nos. 9,327,035 and 8,597,652, U.S. Pub. No. 20140193414, and International Pub. No. WO2010108127.

Exemplary HER3 antibodies also include the HER2/IER3 bispecific antibody MM-111 and any of the HER3 antibodies, whether monospecific or multi-specific, disclosed in U.S. Pub. Nos. 20130183311 and 20090246206 and International Pub. Nos. WO2006091209 and WO2005117973.

According to certain aspects, the HER3 targeting agent includes an anti-HER3 antibody that binds to an epitope of HER3 recognized by Patritumab from Daiichi Sankyo, Seribantumab (MM-121) from Merrimack Pharmaceuticals, Lumretuzumab from Roche, Elgemtumab from Novartis, GSK2849330 from GlaxoSmithKline, CDX-3379 of Celldex Therapeutics, EV20 and MP-RM-1 from MediPharma, Barecetamab (ISU104) from Isu Abxis Co., HMBD-001 (10D1F) from Hummingbird Bioscience Pte., REGN1400 from Regeneron Pharmaceuticals, and/or AV-203 from AVEO Oncology. According to certain aspects, the anti-HER3 antibody is selected from one or more of Patritumab, Seribantumab or an antibody including heavy chain sequence SEQ ID NO:286 and/or light chain sequence SEQ ID NO:287 which are reported for Seribantumab, Lumretuzumab or an antibody including heavy chain sequence SEQ ID NO:288 and/or light chain sequence SEQ ID NO:289 which are reported for Lumretuzumab, Elgemtumab or an antibody including heavy chain sequence SEQ ID NO:290 and/or light chain sequence SEQ ID NO:291 which are reported for Elgemtumab, AV-203, CDX-3379, GSK2849330, EV20, MP-RM-1, ISU104, HM1BD-001 (10D1F), and REGN1400.

An amino acid sequence of the human HER3 precursor protein (receptor tyrosine-protein kinase erbB-3 isoform 1 precursor NCBI Reference Sequence: NP_001973.2) is provided herein as SEQ ID NO:293.

Exemplary CD33 targeting agents that may be radiolabeled, drug-conjugated, or unlabeled for use in the invention include the monoclonal antibodies lintuzumab, gemtuzumab, and vadastuximab. In combination with a radiolabeled CCR8 targeting agent as disclosed herein, a CD33 targeting therapeutic agent may, for example, be used to treat solid cancers, such as ovarian, breast, cervical prostate, gastric, bladder, lung, melanoma, colorectal and squamous cell carcinoma cancers and any of the cancers disclosed herein, for example, by depleting myeloid-derived suppressor cells (MDSCs). In one aspect, the CD33 targeting agent used in combination with a radiolabeled CCR8 targeting agent is ²²⁵Ac-lintuzumab. In another aspect, the CD33 targeting agent used in combination with a radiolabeled HER3 targeting agent is the ADC gemtuzumab ozogamicin (Mylotarg®; Pfizer).

Exemplary CD38 targeting agents that may be radiolabeled, drug-conjugated, or unlabeled for use in the invention include anti-CD38 monoclonal antibodies such as daratumumab (Darzalex®; Johnson and Johnson) and isatuximab (Sarclisa®; Sanofi) or antigen-binding fragments thereof. Such CD38 targeting agents may, for example, be used in combination with the radiolabeled CCR8 targeting agent(s) in the treatment of solid tumors that may, for example, be infiltrated with CD38-positive suppressive immune cells, such as but not limited to ovarian, breast, cervical prostate, gastric, bladder, lung, melanoma, colorectal and squamous cell carcinoma cancers and any of the cancers disclosed herein.

Exemplary MUC1 targeting agents that may be radiolabeled, drug-conjugated, or unlabeled for use in the invention include the monoclonal antibodies: KL-6 (epitope: a sialylated sugar of Krebs von den Lugen-6 (KL-6) PDTRPAP sequence (SEQ ID NO:315); MY1.E12 (epitope: sialyla2-3galactosylh1-3Nacetylgalactosaminide linked to a distinct threonine residue in the MUC1 tandem repeat); 5E5, 2D9 (epitope: Tn or STn in the tandem repeat domain); hMUCl-1H7 (epitope: extracellular domain of MUC1 C-terminal subunit (MUC1-C)); and TAB004 (epitope: STAPPVHNV within the TR sequence (SEQ ID NO:316)); huC242 (epitope: Sialyl-Lewis a epitope CanAg glycoprotein which is similar to MUC1); huPAM4 (epitope: domain located between the amino terminus and start of the repeat domain of a MUC1 antigen (non-VNTR) and also react with MUC5AC); hPAM4 a/k/a Clivatuzumab (epitope: Domain located between the amino terminus and start of the repeat domain of a MUC1 antigen (non-VNTR) and also react with MUC5AC); SAR56665, 8huDS6-DM4 (epitope: O-linked glycans with α2,3-sialylated and β1,4-galactosylated termini in VNTR); Gatipotuzumab (epitope: PDT*RP . . . , (SEQ ID NO:317) where T* is O-glycosylated with GalNAca1—or a similar short, non-sialylated glycan such as Galb1-3GalNAca1-(core-1)); AR20.5 (epitope: DTRPAP (SEQ ID NO: 318) and DTnRPAP (SEQ ID NO:319)), ⁹⁰Y IMMU-107 (hPAM4-Cide; Immunomedics, Inc.; Gilead Sciences, Inc.), or ¹⁷⁷Lu or ²²⁵Ac alternatively labeled versions thereof, hTAB004 (OncoTAb, Inc.) and any of the anti-MUC1 antibodies disclosed in any of U.S. Pub. No. 20200061216 and U.S. Pat. Nos. 8,518,405; 9,090,698; 9,217,038; 9,546,217; 10,017,580; 10,507,251 10,517,966; 10,919,973; 11,136,410; or 11,161,911, antigen-binding fragments of any of said monoclonal antibodies, and antibodies or antigen-binding antibody fragments recognizing the same epitopes as the any of the aforementioned anti-MUC1 antibodies. Such MUC1 targeting agents may, for example, be used in combination with the radiolabeled CCR8 targeting agents in the treatment of solid tumors expressing or overexpressing MUC1, such as pancreatic cancer, breast cancer, such as metastatic breast cancer, tamoxifen-resistant breast cancer, HER2-negative breast cancer, and triple negative breast cancer (TNBC), ovarian cancer, gastric cancer, gastrointestinal cancer, liver cancer such as hepatocellular carcinoma (HCC) and cholangiocarcinoma, and colororectal cancer.

Exemplary LeY targeting agents that may be radiolabeled, drug-conjugated, or unlabeled for use in the invention include but are not limited to monoclonal antibodies such as 3S1931 and/or a humanized version thereof such as Hu3S1933, or any of the monoclonal antibodies B34, BR55-2, BR55/BR96, and IGN 133, or antigen binding fragments of any of the preceding antibodies.

According to certain aspects, the LeY targeting agent may be a monoclonal antibody including a heavy chain variable region having an amino acid sequence as set forth in any one of SEQ ID NOS:297-301. According to certain aspects, the LeY targeting agent may be a monoclonal antibody including a light chain variable region having an amino acid sequence as set forth in SEQ ID NO:302 or 303. According to certain aspects, the LeY targeting agent may be a monoclonal antibody including a heavy chain variable region having the amino acid sequence as set forth in any one of SEQ ID NOS:297-301 and a light chain variable region having the amino acid sequence as set forth in SEQ ID NO:302 or 303.

According to certain aspects, the LeY targeting agent may be a monoclonal antibody including one or more of the heavy chain N-terminal region and complementarity determining regions (CDRs) having amino acid sequences as set forth in SEQ ID NOS:304 and/or 305-307, respectively. According to certain aspects, the LeY targeting agent may be a monoclonal antibody including one or more of the light chain N-terminal region and CDRs having amino acid sequences as set forth in SEQ ID NOS:308 and/or 309-311, respectively. According to certain aspects, the LeY targeting agent may be a monoclonal antibody including one or more of the heavy chain N-terminal region set forth in SEQ ID NO:304 and the heavy chain CDRs set forth in SEQ ID NOS:305-307, and one or more of the light chain N-terminal region set forth in SEQ ID NO:308 and the light chain CDRs having amino acid sequences as set forth in SEQ ID NOS:309-311.

Exemplary LYPD3 (C4.4A) targeting agents that may be radiolabeled, drug conjugated or unlabeled for use in combination or conjunction with a radiolabeled CCR8 targeting agent according to the invention include, for example, BAY 1129980 (a/k/a Lupartumab amadotin; Bayer AG, Germany) an Auristatin-based anti-C4.4A (LYPD3) ADC or its antibody component Lupartumab, IgG₁mAb GT-002 (Glycotope GmbH, Germany) and any of those disclosed in U.S. Pub. No. 20210309711, 20210238292, 20210164985, 20180031566, 20170158775, or 20150030618, 20120321619, Canadian Patent Application No. CA3124332A1, Taiwan Application No. TW202202521A, or Int'l Pub. No. WO2021260208, WO2007044756, WO2022042690, or WO2020138489. Such use may, for example, be for the treatment of a LYPD3-expressing hematological or solid tumor cancer in a mammal, such as carcinomas, primary and metastatic transitional cell carcinomas (TCCs), adenocarcinomas, lung cancer, lung adenocarcinoma, non-small cell lung cancer (NSCLC), hepatocellular carcinoma (HCC), breast cancer, endocrine therapy-resistant breast cancer (such as tamoxifen-resistant breast cancer), HER2-positive breast cancer, triple negative breast cancer (TNBC), esophageal cancer, renal cell carcinomas, colorectal cancer, cervical cancer, head and neck cancer, urothelial cancer, skin cancer, melanoma, and acute myelogenous leukemia (AML).

When the methods include administration of a multi-specific antibody, the first target recognition component may include one of: a first full length heavy chain and a first full length light chain, a first Fab fragment, or a first single-chain variable fragment (scFvs). The second target recognition component may include one of: a second full length heavy chain and a second full length light chain, a second Fab fragment, or a second single-chain variable fragment (scFvs). Moreover, the second target recognition component may be derived from a different epitope of the CCR8 antigen or may be derived from any of the antigens listed above.

In one aspect, the CCR8 targeting agent includes a radioisotope, and any additional antibodies against other antigens may optionally include a radioisotope. When the immunotherapy includes a bispecific antibody, either one or both of the first target recognition component and the second target recognition component may include a radioisotope.

A related aspect of the invention provides a radiopharmaceutical composition that includes a radiolabeled CCR8 targeting agent, such as an antibody, and one or more different therapeutically active targeting agents, such as one or more antibodies, directed against different (non-CCR8) target antigens, such as any of those disclosed herein, each of which different targeting agents may be radiolabeled, drug-conjugated (cytotoxin conjugated), or unconjugated (i.e., neither radio- or drug-conjugated), for the treatment of a cancer such as a solid tumor or any of the cancers disclosed herein, in a mammal such as human patient, The radiolabeled CCR8 targeting agent, such as an antibody, and the one or more different targeting agents, such as one or more antibodies, directed against different (non-CCR8) target antigens, may together be present in therapeutically effective amounts in the composition. The composition may, for example, further include one or more pharmaceutically acceptable excipients or carriers. Such a composition may be in the form of a liquid suitable for injection, such as for intravenous administration.

The radioconjugated CCR8 targeting agent may, for example, exhibit essentially the same reactivity (e.g., immunoreactivity) to the antigen as a control targeting agent, wherein the control targeting agent includes an unlabeled targeting agent against the same epitope of the antigen (i.e., CCR8) as the radiolabeled targeting agent.

The CCR8 targeting agent such as monoclonal antibody may, for example, be labeled with ²²⁵Ac and may be at least 5-fold more effective at causing cell death of CCR8-positive cells than a control targeting agent/monoclonal antibody, wherein the control monoclonal antibody includes an unlabeled antibody against the same epitope of the antigen as the ²²⁵Ac labeled antibody. For example, a ²²⁵Ac labeled monoclonal antibody may be at least 10-fold more effective, at least 20-fold more effective, at least 50-fold more effective, or at least 100-fold more effective at causing cell death of CCR8-positive cells than the control monoclonal antibody.

The methods may include administration of radiolabeled and unlabeled (non-radiolabeled) fractions/portions of the CCR8 targeting agent, such as an antibody, antibody fragment, etc. For example, the unlabeled fraction may include the same antibody against the same epitope as the labeled fraction. In this way, the total radioactivity of the antibody may be varied or may be held constant while the overall antibody protein concentration may be held constant or may be varied, respectively. For example, the total protein concentration of unlabeled antibody fraction administered may vary depending on the exact nature of the disease to be treated, age and weight of the patient, identity of the monoclonal antibody, and the label (e.g., radionuclide) selected for labeling of the monoclonal antibody. Similarly, where multiple separate radiolabeled targeting agents are administered to a subject, whether in the same composition or separate compositions, each of the targeting agents may be present in the/its composition in a radiolabeled fraction/portion and a non-radiolabeled fraction/portion.

The effective amount of the radioconjugated CCR8 targeting agent may, for example, be a maximum tolerated dose (MTD) of the radioconjugated CCR8 targeting agent, such as an antibody against CCR8.

When more than one CCR8 targeting agent, other targeting agent, or other immunotherapy is administered, the agents/antibodies may, for example, be administered at the same time. As such, the agents/antibodies may be provided in a single composition or as separate compositions. Alternatively, the two agents/antibodies may be administered sequentially. As such, the radioconjugated CCR8 targeting agent may be administered before the second agent/antibody, after the second agent/antibody, or both before and after the second agent/antibody. Moreover, the second agent/antibody may, for example, be administered before the radioconjugated CCR8 targeting agent, after the radioconjugated CCR8 targeting agent, or both before and after the radioconjugated CCR8 targeting agent.

The radioconjugated CCR8 targeting agent may, for example, be administered according to a dosing schedule selected from the group consisting of one every 7, 10, 12, 14, 20, 24, 28, 35, and 42 days throughout a treatment period, wherein the treatment period includes at least two doses.

The radioconjugated CCR8 targeting agent may, for example, be administered according to a dose schedule that includes 2 doses, such as on days 1 and 5, 6, 7, 8, 9, or 10 of a treatment period, or days 1 and 8 of a treatment period.

Administration of the radioconjugated CCR8 targeting agent(s) of the present disclosure, in addition to other therapeutic agents, may be provided in a number of ways depending upon whether local or systemic treatment is desired and upon the area to be treated. Administration may, for example, be intratracheal, intranasal, epidermal and transdermal, oral or parenteral. Parenteral administration includes intravenous, intra-arterial, subcutaneous, intraperitoneal or intramuscular injection or infusion; or intracranial, e.g., intrathecal or intraventricular, administration. In some embodiments a slow-release preparation including the targeting agents(s) and/or other therapeutic agents may be administered. The various agents may, for example, be administered as a single treatment or in a series of treatments that continue as needed and for a duration of time that causes one or more symptoms of the cancer to be reduced or ameliorated, or that achieves another desired effect.

The dose(s) may vary, for example, depending upon the identity, size, and condition of the subject, further depending upon the route by which the composition is to be administered and the desired effect. Appropriate doses of a therapeutic agent depend upon the potency with respect to the expression or activity to be modulated. The therapeutic agents may, for example, be administered to a mammal (e.g., a human) at a relatively low dose at first, with the dose subsequently increased until an appropriate response is obtained.

The radioconjugated CCR8 targeting agent may, for example, be administered simultaneously or sequentially with one or more additional therapeutic agents or modalities, such as any of those described herein. Moreover, when more than one additional therapeutic agent is included, the additional therapeutic agents may be administered simultaneously or sequentially with each other and/or with the radioconjugated CCR8 targeting agent. It should further be understood that administration of a radiolabeled CCR8 targeting agent in conjunction with another therapeutic agent or modality may be temporally overlapping such that at least part but not necessarily all of the administration of the two therapies is performed simultaneously. Those skilled in the art will appreciate that when different therapeutic agents are administered sequentially to a subject, their biological/therapeutic activity and/or effects (or those of an active metabolite, if applicable) may nevertheless be present within the subject in a temporally overlapping manner.

### Radiolabeling the CCR8 Targeting Agent

The human CCR8 targeting agents of the present disclosure are labeled with a radioisotope. The CCR8 targeting agent may, for example, be an antibody against CCR8 that is deglycosylated in the constant region, such as at asparagine-297 (Asn-297, N297; kabat number) in the heavy chain CH2 domain, for the purpose of uncovering a unique conjugation site, glutamine (i.e., Gln-295, Q295) so that it is available for conjugation with bifunctional chelator molecules which may then chelate the radionuclide.

The CCR8 targeting agent may, for example, be an antibody against CCR8 that may have reduced disulfide bonds such as by using reducing agents, which may then be converted to dehydroalanine for the purpose of conjugating with a bifunctional chelator molecule.

The CCR8 targeting agent may, for example, be an antibody against CCR8 that may have reduced disulfide bonds, such as by use of reducing agents, followed by conjugation via aryl bridges with a bifunctional chelator molecule. For example, a linker molecule such as 3,5-bis(bromomethyl)benzene may bridge the free sulfhydryl groups on the CCR8 targeting agent.

The CCR8 targeting agent may, for example, be an antibody against CCR8 that may have certain specific existing amino acids replaced with cysteine(s) that then can be used for site-specific labeling (e.g., conjugation with a bifunctional chelator molecule which may then chelate the radionuclide).

The CCR8 targeting agents may, for example, be radiolabeled through site-specific conjugation of suitable bifunctional chelators. Exemplary chelator molecules that may be used include p-SCN-Bn-DOTA, NH₂-DOTA, NH₂—(CH₂)₁₋₂₀-DOTA, NH₂-(PEG)₁₋₂₀-DOTA, HS-DOTA, HS—(CH₂)₁₋₂₀-DOTA, HS-(PEG)₁₋₂₀-DOTA, dibromo-S—(CH₂)₁₋₂₀-DOTA, dibromo-S-(PEG)₁₋₂₀-DOTA, p-SCN-Bn-DOTP, NH₂-DOTP, NH₂—(CH₂)₁₋₂₀-DOTP, NH₂-(PEG)₁₋₂₀-DOTP, HS-DOTP, HS—(CH₂)₁₋₂₀-DOTP, HS-(PEG)₁₋₂₀-DOTP, dibromo-S—(CH₂)₁₋₂₀-DOTP, and dibromo-S-(PEG)₁₋₂₀-DOTP.

The chelator molecules may, for example, be attached to the CCR8 targeting agent through a linker molecule.

Methods for conjugation and chelation of an exemplary radionuclide are discussed in more detail in Example 1.

### Diagnostics

The presently disclosed methods may, for example, include diagnosing the subject to ascertain if CCR8-positive cells (i.e., CCR8 expressing Tregs) are present in the tumor microenvironment, such as CCR8-positive cells present in a tumor biopsy from the subject, or present in the patient or tumors in general. The diagnosing step may, for example, include obtaining a sample of tissue from the subject and mounting the sample on a substrate. The presence, absence, and/or extent of the CCR8 antigen may, for example, be detected using a diagnostic antibody, peptide, or small molecule, wherein the diagnostic antibody peptide, or small molecule is labeled with any of the standard detectable or imaging labels known in the art. Exemplary labeling agents include radiolabels such as ³H, ¹⁴C, ³²P ³⁵S, and ¹²⁵I; fluorescent or chemiluminescent compounds, such as fluorescein isothiocyanate, rhodamine, or luciferin; and enzymes, such as alkaline phosphatase, β-galactosidase, or horseradish peroxidase. An exemplary CCR8 targeting agent used in such a diagnostic assay includes a human or humanized antibody against CCR8 or a CCR8-binding antibody fragment.

Alternatively or in addition, a determination as to whether, to what extent, and/or where (localization) CCR8-positive cells are present in a patient may be made using a CCR8 targeting agent labeled with any of ¹⁸F, ¹¹C, ⁶⁸Ga, ⁶⁴Cu, ⁸⁹Zr, ¹²⁴, ⁴⁴Sc, or ⁸⁶Y, for PET imaging, or ⁶⁷Ga, ^{99m}Tc, ¹¹¹In or ¹⁷⁷Lu, for SPECT imaging. Accordingly, the method may include administering to the subject a CCR8 targeting agent labeled with one or more of ¹⁸F, ¹¹C, ⁶⁸Ga, ⁶⁴Cu, ⁸⁹Zr, ¹²⁴, ⁴⁴Sc, ⁸⁶Y, ^{99m}Tc, ¹⁷⁷Lu, or ¹¹¹In, and performing a non-invasive imaging technique on the subject, such as performing a PET or SPECT scan on the subject. The method may include, performing the imaging after a sufficient amount of time, such as at least 15 minutes, at least 30 minutes, at least 60 minutes, at least, 90 minutes, or at at least 120 minutes, has elapsed from the administration of the labeled CCR8 targeting agent for said agent to distribute to tissues of the subject and bind CCR8 that may be present therein. The CCR8 targeting agent may include any of ¹⁸F, ¹¹C, ⁶⁸Ga, ⁶⁴Cu, ⁸⁹Zr, 124I, ⁴⁴Sc, ⁸⁶Y, ^{99m}Tc, ¹⁷⁷Lu, or ¹¹¹In, such as any of ⁶⁸Ga, ⁸⁹Zr, or 111In, and may be labeled using any of the methods disclosed herein (e.g., such as disclosed in Example 1).

If the subject has CCR8-positive cells, for example, beyond a predetermined threshold level, the therapeutic methods of the present disclosure may be carried out, i.e., administration of a therapeutically effective amount of a radioconjugated CCR8 targeting agent (e.g., ²²⁵Ac conjugated CCR8 targeting agent), either alone or in combination with one or more additional therapeutic agents or modalities.

### Additional Therapeutic Agents and Modalities

The methods of the present disclosure, which include administration of a radioconjugated CCR8 targeting agent, may further include administration of an additional therapeutic agent and/or modality. The additional agent and/or modality may be relevant to, e.g., active against, the disease or condition being treated. Such administration may be simultaneous, separate or sequential with the administration of the effective amount of the radioconjugated CCR8 targeting agent. For simultaneous administration, the agents may be administered as one composition, or as separate compositions, as appropriate.

Exemplary additional therapeutic agents and modalities include at least chemotherapeutic agents, anti-inflammatory agents, immunosuppressive agents, immune-modulatory agents, external beam radiation, or any combination thereof. Further exemplary additional agents also include immune checkpoint therapies or blockades, MICA blockades, DDR inhibitors, CD47 blockades, adoptive cell therapy, and any combination thereof.

### A. Chemotherapeutic Agents

Exemplary chemotherapeutic agents that may be used include, but are not limited to, anti-neoplastic agents including alkylating agents including: nitrogen mustards, such as mechlorethamine, cyclophosphamide, ifosfamide, melphalan and chlorambucil; nitrosoureas, such as carmustine (BCNU), lomustine (CCNU), and semustine (methyl-CCNU); Temodal™ (temozolomide), ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil (5FU), fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguamne, azathioprine, T-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; pipodophylotoxins such as etoposide and teniposide; antibiotics such as actinomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as oxaliplatin, cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o, p-DDD) and aminoglutethimide; hormones and antagonists including adrenocorticosteroid antagonists such as prednisone and equivalents, dexamethasone and aminoglutethimide; Gemzar™ (gemcitabine), progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide. Therapies targeting epigenetic mechanism including, but not limited to, histone deacetylase inhibitors, demethylating agents (e.g., Vidaza®) and release of transcriptional repression (ATRA) therapies can also be combined with antibodies of the invention.

The chemotherapeutic agents may, for example, include at least a radiosensitizer, such as temozolomide, cisplatin, and/or fluorouracil.

The chemotherapeutic agents may, for example, be administered according to any standard dose regime known in the field. For example, chemotherapeutic agents may be administered at concentrations in the range of 1 to 500 mg/m², the amounts being calculated as a function of patient surface area (m²). For example, exemplary doses of the chemotherapeutic paclitaxel may include 15 mg/m² to 275 mg/m², exemplary doses of docetaxel may include 60 mg/m² to 100 mg/m², exemplary doses of epithilone may include 10 mg/m² to 20 mg/m², and an exemplary dose of calicheamicin may include 1 mg/m² to 10 mg/m². While exemplary doses are listed herein, such are only provided for reference and are not intended to limit the dose ranges of the drug agents of the present disclosure.

B. External Beam Radiation and/or Brachytherapy

The additional therapeutic modality administered with the radioconjugated CCR8 targeting agent and optionally any other of the other additional therapeutics disclosed herein may, for example, include ionizing radiation, such as administered via external beam radiation or brachytherapy. Such radiation generally refers to the use of X-rays, gamma rays, or charged particles (e.g., protons or electrons) to generate ionizing radiation, such as delivered by a machine placed outside the patient's body (external-beam radiation therapy) or by a source placed inside a patient's body (internal radiation therapy or brachytherapy).

Such external beam radiation or brachytherapy may enhance the targeted radiation damage delivered by the radioconjugated CCR8 targeting agent and may thus be delivered sequentially with the radioconjugated CCR8 targeting agent, such as before and/or after the radioconjugated CCR8 targeting agent, or simultaneous with the radioconjugated CCR8 targeting agents.

The external beam radiation or brachytherapy may be planned and administered in conjunction with imaging-based techniques such as computed tomography (CT) and/or magnetic resonance imaging (MRI) to accurately determine the dose and location of radiation to be administered. For example, a patient treated with any of the radioconjugated CCR8 targeting agents disclosed herein may be imaged using either of CT or MRI to determine the dose and location of radiation to be administered by the external beam radiation or brachytherapy.

The radiation therapy may be selected from the group consisting of total all-body radiation therapy, conventional external beam radiation therapy, stereotactic radiosurgery, stereotactic body radiation therapy, 3-D conformal radiation therapy, intensity-modulated radiation therapy, image-guided radiation therapy, tomotherapy, and brachytherapy. The radiation therapy may be provided as a single dose or as fractionated doses, e.g., as 2 or more fractions. For example, the dose may be administered such that each fraction includes 2-20 Gy (e.g., a radiation dose of 50 Gy may be split up into 10 fractions, each including 5 Gy). The 2 or more fractions may be administered on consecutive or sequential days, such as once in 2 days, once in 3 days, once in 4 days, once in 5 days, once in 6 days, once in 7 days, or in any combination thereof.

### C. Immune Checkpoint Therapies

The additional agent(s) administered with the radioconjugated CCR8 targeting agent may, for example, include an immune checkpoint therapy. Cancer cells have developed means to evade the standard checkpoints of the immune system. For example, cancer cells have been found to evade immunosurveillance through reduced expression of tumor antigens, downregulation of MHC class I and II molecules leading to reduced tumor antigen presentation, secretion of immunosuppressive cytokines such as TGFb, recruitment or induction of immunosuppressive cells such as regulatory T cells (Treg) or myeloid-derived suppressor cells (MDSC), and overexpression of certain ligands [e.g., programmed death ligand-1 (PD-L1)] that inhibit the host's existing antitumor immunity.

Another major mechanism of immune suppression by cancer cells is a process known as “T cell exhaustion”, which results from chronic exposure to tumor antigens, and is characterized by the upregulation of inhibitory receptors. These inhibitory receptors serve as immune checkpoints in order to prevent uncontrolled immune reactions.

Various immune checkpoints acting at different levels of T cell immunity that have been described in the literature may be used, including PD-1 (i.e., programmed cell death protein 1) and its ligands PD-L1 and PD-L2, CTLA-4 (i.e., cytotoxic T-lymphocyte associated protein-4) and its ligands CD80 and CD86, LAG3 (i.e., Lymphocyte-activation gene 3), B and T lymphocyte attenuator, TIGIT (T cell immunoreceptor with Ig and ITIM domains), TIM-3 (i.e., T cell immunoglobulin and mucin-domain containing protein 3), and VISTA (V-domain immunoglobulin suppressor of T cell activation).

Enhancing the efficacy of the immune system by therapeutic intervention is a particularly exciting development in cancer treatment. As indicated, checkpoint inhibitors such as CTLA-4 and PD-1 prevent autoimmunity and generally protect tissues from immune collateral damage. In addition, stimulatory checkpoints, such as OX40 (i.e., tumor necrosis factor receptor superfamily, member 4; TNFR-SF4), CD137 (i.e., TNFR-SF9), GITR (i.e., Glucocorticoid-Induced TNFR), CD27 (i.e., TNFR-SF7), CD40 (i.e., cluster of differentiation 40), and CD28, activate and/or promote the expansion of T cells. Regulation of the immune system by inhibition or overexpression of these proteins in combination with the radioconjugated CCR8 targeting agents may provide synergistic therapeutic responses and enhanced therapeutic methods.

Thus, a promising therapeutic strategy of this disclosure is the use of immune checkpoint therapies to remove certain blockades on the immune system utilized by cancer cells, in combination with the radioconjugated CCR8 targeting agents disclosed herein. For example, antibodies against certain immune checkpoint inhibitors may block interaction between checkpoint inhibitor proteins and their ligands, therefore preventing the signaling events that would otherwise have led to inhibition of an immune response against the tumor cell.

Moreover, there is a growing body of preclinical evidence supporting the ability of radiation to synergize with immune checkpoint inhibitor antibodies, and this is also being explored in the clinic with increasing numbers of clinical trials evaluating the combination of external beam radiation with immune checkpoint therapies across various tumor types and immune checkpoint inhibitor antibodies (Lamichhane, 2018). Clinical evidence supporting this combination has been generated in melanoma, with two studies demonstrating a clinical benefit using radiation in combination with the anti-cytotoxic T-lymphocyte-associated protein 4 (CTLA-4) immune checkpoint inhibitor antibody, Ipilimumab (Twyman-Saint Vistor, 2015; Yervoy® Bristol Myers Squibb).

Accordingly, one object of the present disclosure is to provide therapies for the treatment of cancer using a radioconjugated CCR8 targeting agent in combination with one or more immune checkpoint therapies, such as an inhibitor of an immune checkpoint protein.

Immune checkpoint therapies that may be employed include molecules that totally or partially reduce, inhibit, interfere with or modulate one or more checkpoint proteins, such as checkpoint proteins that regulate T cell activation or function. Immune checkpoint therapies may unblock an existing immune response inhibition by binding to or otherwise disabling checkpoint inhibition. The immune checkpoint therapy may include monoclonal antibodies, humanized antibodies, fully human antibodies, antibody fragments, peptides, small molecule therapeutics, or any combination thereof.

Exemplary immune checkpoint therapies that may be used include antibodies, peptides, and small molecules that may bind to and inhibit a checkpoint protein, such as the inhibitory receptors CTLA-4, PD-1, TIM-3, VISTA, BTLA, LAG-3 and TIGIT. Additionally, the immune checkpoint therapies include antibodies, peptides, and small molecules that may bind to a ligand of any of the aforementioned checkpoint proteins, such as PD-L1, PD-L2, PD-L3, and PD-L4 (ligands for PD-1) and CD80 and CD86 (ligands for CTLA-4). Other exemplary immune checkpoint therapies may bind to checkpoint proteins such as the activating receptors CD28, OX40, CD40, GITR, CD137, CD27, and HVEM, or ligands thereof (e.g., CD137-L and GITR-L), CD226, B7-H3, B7-H4, BTLA, TIGIT, GALS, KIR, 2B4 (belongs to the CD2 family of molecules and is expressed on all NK, T6, and memory CD8+ (αβ) T cells), CD160 (also referred to as BY55), and CGEN-15049.

The CTLA-4 and PD-1 pathways are thought to operate at different stages of an immune response. CTLA-4 is considered the “leader” of the immune checkpoint inhibitors, as it stops potentially autoreactive T cells at the initial stage of naive T cell activation, typically in lymph nodes. The PD-1 pathway regulates previously activated T cells at the later stages of an immune response, primarily in peripheral tissues. Moreover, progressing cancer patients have been shown to lack upregulation of PD-L1 by either tumor cells or tumor-infiltrating immune cells. Immune checkpoint therapies targeting the PD-1 pathway might thus be especially effective in tumors where this immune suppressive axis is operational and reversing the balance towards an immune protective environment would rekindle and strengthen a pre-existing anti-tumor immune response. PD-1 blockade can be accomplished by a variety of mechanisms including antibodies that bind PD-1 or its ligand, PD-L1.

Accordingly, the immune checkpoint therapy may, for example, include an inhibitor of the PD-1 checkpoint, which may decrease, block, inhibit, abrogate, or interfere with signal transduction resulting from the interaction of PD-1 with one or more of its binding partners, such as PD-L1 and PD-L2. The inhibitor of the PD-1 checkpoint may be an anti-PD-1 antibody, antigen binding fragment, fusion proteins, oligopeptides, and other molecules that decrease, block, inhibit, abrogate or interfere with signal transduction resulting from the interaction of PD-1 with PD-L1 and/or PD-L2. The PD-1 checkpoint inhibitor may reduce the negative co-stimulatory signal mediated by or through cell surface proteins expressed on T lymphocytes so as render a dysfunctional T cell less dysfunctional (e.g., enhancing effector responses to antigen recognition). The PD-1 checkpoint therapy may be an anti-PD-1 antibody.

The immune checkpoint therapy may, for example, include an antibody against PD-1 such as nivolumab, or any of the inhibitors of PD-1 biological activity (or its ligands) disclosed in U.S. Pat. No. 7,029,674. Additional exemplary antibodies against PD-1 include: Anti-mouse PD-1 antibody Clone J43 (Cat #BE0033-2) from BioXcell; Anti-mouse PD-1 antibody Clone RMP1-14 (Cat #BE0146) from BioXcell; mouse anti-PD-1 antibody Clone EH12; Merck's MK-3475 anti-mouse PD-1 antibody (Keytruda®, pembrolizumab, lambrolizumab); and AnaptysBio's anti-PD-1 antibody, known as ANBO11; antibody MDX-1 106 (ONO-4538); Bristol-Myers Squibb's human IgG4 monoclonal antibody nivolumab (Opdivo®, BMS-936558, MDX1106); AstraZeneca's AMP-514, and AMP-224; and Pidilizumab (CT-011), CureTech Ltd.

The immune checkpoint therapy may, for example, include an inhibitor of PD-L1 such as an antibody (e.g., an anti-PD-L1 antibody, i.e., ICI antibody), RNAi molecule (e.g., anti-PD-L1 RNAi), antisense molecule (e.g., an anti-PD-L1 antisense RNA), dominant negative protein (e.g., a dominant negative PD-L1 protein), and/or small molecule inhibitor. An exemplary anti-PD-L1 antibody includes clone EH12, or any of Genentech's MPDL3280A (RG7446); anti-mouse PD-L1 antibody Clone 10F.9G2 (Cat #BE0101) from BioXcell; anti-PD-L1 monoclonal antibody MDX-1105 (BMS-936559) and BMS-935559 from Bristol-Meyer's Squibb; MSB0010718C; mouse anti-PD-L1 Clone 29E.2A3; and AstraZeneca's MEDI4736 (Durvalumab).

The immune checkpoint therapy may, for example, include an inhibitor of PD-L2 or may reduce the interaction between PD-1 and PD-L2. Exemplary inhibitors of PD-L2 include antibodies (e.g., an anti-PD-L2 antibody, i.e., ICI antibody), RNAi molecules (e.g., an anti-PD-L2 RNAi), antisense molecules (e.g., an anti-PD-L2 antisense RNA), dominant negative proteins (e.g., a dominant negative PD-L2 protein), and small molecule inhibitors. Antibodies include monoclonal antibodies, humanized antibodies, deimmunized antibodies, and Ig fusion proteins.

The immune checkpoint therapy may, for example, include an inhibitor of CTLA-4, such as an antibody against CTLA-4. An exemplary antibody against CTLA-4 includes ipilimumab. The anti-CTLA-4 antibody may block the binding of CTLA-4 to CD80 (B7-1) and/or CD86 (B7-2) expressed on antigen presenting cells. Exemplary antibodies against CTLA-4 further include: Bristol Meyers Squibb's anti-CTLA-4 antibody ipilimumab (also known as Yervoy®, MDX-010, BMS-734016 and MDX-101); anti-CTLA4 Antibody, clone 9H10 from Millipore; Pfizer's tremelimumab (CP-675,206, ticilimumab); and anti-CTLA-4 antibody clone BNI3 from Abcam. The immune checkpoint inhibitor may be a nucleic acid inhibitor of CTLA-4 expression.

The immune checkpoint therapy may, for example, include an inhibitor of LAG3. Lymphocyte activation gene-3 (LAG3) functions as an immune checkpoint in mediating peripheral T cell tolerance. LAG3 (also called CD223) is a transmembrane protein receptor expressed on activated CD4 and CD8 T cells, 76 T cells, natural killer T cells, B-cells, natural killer cells, plasmacytoid dendritic cells and regulatory T cells. The primary function of LAG3 is to attenuate the immune response. LAG3 binding to MHC class II molecules results in delivery of a negative signal to LAG3-expressing cells and down-regulates antigen-dependent CD4 and CD8 T cell responses. Thus, LAG3 negatively regulates the ability of T cells to proliferate, produce cytokines, and lyse target cells, termed as ‘exhaustion’ of T cells, and inhibition of LAG3 function may enhance T cell proliferation.

Monoclonal antibodies to LAG3 that may be used are known in the art and described, for example, in U.S. Pat. Nos. 5,976,877, 6,143,273, 6,197,524, 8,551,481, 10,898,571, and U.S. Appl. Pub. Nos. 20110070238, 20110150892, 20130095114, 20140093511, 20140127226, 20140286935, and in WO95/30750, WO97/03695, WO98/58059, WO2004/078928, WO2008/132601, WO2010/019570, WO2014/008218, EP0510079B1, EP0758383B1, EP0843557B1, EP0977856B1, EP1897548B2, EP2142210A1, and EP2320940B1. The LAG-3 inhibitor used may, for example, include Relatlimab (Bristol Myers Squibb). Additionally, peptide inhibitors of LAG3 which may be used are also known and described in U.S. Pub. No. 20200369766.

The immune checkpoint therapy may, for example, include an inhibitor of the TIM3 protein. T-cell immunoglobulin and mucin-domain containing-3 (TIM3), also known as hepatitis A virus cellular receptor 2 (HAVCR2), is a type-I transmembrane protein that functions as a key regulator of immune responses. TIM3 has been shown to induce T cell death or exhaustion after binding to galectin-9, and to play an important in regulating the activities of many innate immune cells (e.g., macrophages, monocytes, dendritic cells, mast cells, and natural killer cells; Han, 2013). Like many inhibitory receptors (e.g., PD-1 and CTLA-4), TIM3 expression has been associated with many types of chronic diseases, including cancer. TIM3+ T cells have been detected in patients with advanced melanoma, non-small cell lung cancer, or follicular B-cell non-Hodgkin lymphoma. And the presence of TIM3+ regulatory T cells have been described as an effective indicator of lung cancer progression. Thus, inhibition of TIM3 may enhance the functions of innate immune cells. Exemplary TIM3 inhibitors include antibodies, peptides, and small molecules that bind to and inhibit TIM3.

The immune checkpoint therapy may, for example, include an inhibitor of the VISTA protein. The V-domain Ig suppressor of T cell activation (VISTA or PD-L3) is primarily expressed on hematopoietic cells, and its expression is highly regulated on myeloid antigen-presenting cells (APCs) and T cells. Expression of VISTA on antigen presenting cells (APCs) suppresses T cell responses by engaging its counter-receptor on T cells during cognate interactions between T cells and APCs. Inhibition of VISTA would enhance T cell-mediated immunity and anti-tumor immunity, suppressing tumor growth. In this regard, therapeutic intervention of the VISTA inhibitory pathway represents a novel approach to modulate T cell-mediated immunity, such as in combination with the presently disclosed radioconjugated CCR8 targeting agents.

The immune checkpoint therapy may, for example, include more than one modulator of the same or different immune checkpoint proteins. As such, the immune checkpoint therapy may include a first antibody or inhibitor against a first immune checkpoint protein and a second antibody or inhibitor against a second immune checkpoint protein.

### D. MICA Blockade

The additional agents administered with the radioconjugated CCR8 targeting agent may include a therapeutic that binds to MHC class I chain-related molecule A (MICA) to allow stabilization of surface expression of MICA on cancer cells for enhanced NK cell-mediated cytolysis.

Cancer cells are known to secrete MHC class I chain-related molecule A (MICA) and MICB, which activate cytotoxicity by lymphocytes and NK cells through their Natural Killer Group 2D (NKG2D) receptor as a mechanism of immunological defense. These induced-self proteins are absent or present at very low levels on the surface of normal cells, but can be expressed in increased amounts in infected, transformed, senescent, and stressed cells. For example, MICA and MICB are expressed by various tumors, including those of epithelial origin (Paschen, 2009) and leukemias (Kato, 2007).

The recognition of the MICA and MICB ligands on tumor cells by the NKG2D receptor, found on NK cells, induces the cytotoxic activity of NK cells (Santoni, 2007) and the subsequent lysis of their tumor targets (Papazahariadou, 2007). The secretion of MICA and MICB by cancer cells has been suggested as a mechanism for tumor cell immune escape through the saturation of NKG2D receptors on cytotoxic cells, thus abrogating their ability to recognize tumor cells. Antibodies targeting MICA have been shown to block the MICA/NKG2D interaction and mediate complement-dependent cytotoxicity (CDC) and antibody-dependent cell cytotoxicity (ADCC) toward MICA expressing cells.

Exemplary anti-MICA antibodies that may be used include at least clone IPH43 or HYB3-24302 from Creative Biolabs, or anti-MICA antibodies against the alpha-3 domain, such as clone 7C6 reported in U.S. Pub. No. 20200165343.

### E. DNA Damage Response Inhibitors

The additional agents administered with the radioconjugated CCR8 targeting agent may include a DNA damage response inhibitor (DDRi). DNA damage can be due to endogenous factors, such as spontaneous or enzymatic reactions, chemical reactions, or errors in replication, or may be due to exogenous factors, such as UV or ionizing radiation or genotoxic chemicals. The repair pathways that overcome this damage are collectively referred to as the DNA damage response or DDR. This signaling network acts to detect and orchestrate a cell's response to certain forms of DNA damage, most notably double strand breaks and replication stress. Following treatment with many types of DNA damaging drugs and ionizing radiation, cells are reliant on the DDR for survival. It has been shown that disruption of the DDR can increase cancer cell sensitivity to these DNA damaging agents and thus may improve patient responses to such therapies.

Within the DDR, there are several DNA repair mechanisms, including base excision repair, nucleotide excision repair, mismatch repair, homologous recombinant repair, and non-homologous end joining. Approximately 450 human DDR genes code for proteins with roles in physiological processes. Dysregulation of DDR leads to a variety of disorders, including genetic, neurodegenerative, immune, cardiovascular, and metabolic diseases or disorders and cancers. For example, the genes OGG1 and XRCC1 are part of the base excision repair mechanism of DDR, and mutations in these genes are found in renal, breast, and lung cancers, while the genes BRCA1 and BRCA2 are involved in homologous recombination repair mechanisms and mutations in these genes leads to an increased risk of breast, ovarian, prostate, pancreatic, as well as gastrointestinal and hematological cancers, and melanoma. Exemplary DDR genes are provided in Table 1.

The methods disclosed herein may include administration of the radioconjugated CCR8 targeting agents to deliver ionizing radiation in combination with a DDRi. Thus, the additional agent(s) administered with the radioconjugated CCR8 targeting agent may target proteins in the DDR, i.e., DDR inhibitors or DDRi, thus maximizing DNA damage or inhibiting repair of the damage, such as in G1 and S-phase and/or preventing repair in G2, ensuring the maximum amount of DNA damage is taken into mitosis, leading to cell death.

TABLE 1

| DNA repair | Gene | |
|---|---|---|
| mechanism | examples | Cancer |
| Base Excision | OGG1 | Renal, breast and lung cancer |
| Repair | XRCC1 | Non-small cell lung cancer |
| Nucleotide | ERCC1 | Lung and skin cancer, and glioma |
| Excision | XP | Xeroderma pigmentosum predisposing |
| Repair | | to skin cancer. Also increased risk |
| | | of bladder and lung cancer |
| Mismatch | MSH2, | Lynch syndrome predisposing to |
| Repair | MLH1 | colorectal cancer as well as |
| | | endometrial, ovarian, stomach, |
| | | small intestine, hepatobiliary |
| | | tract, upper urinary tract, brain |
| | | and skin cancer |
| Homologous | BRCA1, | Increased risk of breast, ovarian, |
| Recombinant | BRCA2 | prostate, pancreatic, as well as |
| Repair | | gastrointestinal and hematological |
| | | cancer, and melanoma |
| Non-homologous | KU70 | Breast, colorectal and lung cancer |
| End Joining | KU80 | Lung cancer |
| Cell cycle | ATM | Ataxia-telangiectasia predisposing to |
| checkpoints | | leukemia, breast and pancreatic cancer |
| | ATR | Leukemia, lymphoma, gastric and |
| | | endometrial cancer |

Moreover, one or more DDR pathways may be targeted to ensure cell death, i.e., lethality to the targeted cancer cells. For example, mutations in the BRCA1 and 2 genes alone may not be sufficient to ensure cell death, as other pathways, such as the PARP1 base excision pathway, may act to repair the DNA damage. Thus, combinations of multiple DDRi inhibitors or combining DDRi with antiangiogenic agents or immune checkpoint inhibitors, such as listed hereinabove, may also be used according to the invention.

### Exemplary DDRi—ATM and ATR Inhibitors

Ataxia telangiectasia mutated (ATM) and Ataxia talangiectasia mutated and Rad-3 related (ATR) are members of the phosphatidylinositol 3-kinase-related kinase (PIKK) family of serine/threonine protein kinases.

ATM is a serine/threonine protein kinase that is recruited and activated by DNA double-strand breaks. The ATM phosphorylates several key proteins that initiate activation of a DNA damage checkpoint, leading to cell cycle arrest, DNA repair, or cellular apoptosis. Several of these targets, including p53, CHK2, and H2AX, are tumor suppressors. The protein is named for the disorder ataxia telangiectasia caused by mutations of the ATM. The ATM belongs to the superfamily of phosphatidylinositol 3-kinase-related kinases (PIKKs), which includes six serine/threonine protein kinases that show a sequence similarity to a phosphatidylinositol 3-kinase (PI3K).

Like ATM, ATR is one of the central kinases involved in the DDR. ATR is activated by single stranded DNA structures, which may for example arise at resected DNA DSBs or stalled replication forks. When DNA polymerases stall during DNA replication, the replicative helicases continue to unwind the DNA ahead of the replication fork, leading to the generation of long stretches of single stranded DNA (ssDNA).

ATM has been found to assist cancer cells by providing resistance against chemotherapeutic agents and thus favors tumor growth and survival. Inhibition of ATM and/or ATR may markedly increase cancer cell sensitivity to DNA damaging agents, such as the ionizing radiation provided by the radiolabeled CCR8 targeting agent. Accordingly, one object of the present disclosure includes administration of an inhibitor of ATM (ATMi) and/or ATR (ATRi), in combination or conjunction with administration of the CCR8 targeting agent(s), to inhibit or kill cancer cells, such as those expressing tor overexpressing CCR8.

The inhibitor of ATM (ATMi) or ATR (ATRi) used may, for example, be an antibody, peptide, or small molecule that targets ATM or ATR, respectively. Alternatively, an ATMi or ATRi may reduce or eliminate activation of ATM or ATR by one or more signaling molecules, proteins, or other compounds, or can result in the reduction or elimination of ATM or ATR activation by all signaling molecules, proteins, or other compounds. ATMi and/or ATRi also include compounds that inhibit their expression (e.g., compounds that inhibit ATM or ATR transcription or translation). An exemplary ATMi KU-55933 suppresses cell proliferation and induces apoptosis. Other exemplary ATMi include at least KU-59403, wortmannin, CP466722, and KU-60019. Exemplary ATRi include at least Schisandrin B, NU6027, NVP-BEA235, VE-821, VE-822, AZ20, and AZD6738.

### Exemplary DDRi—Wee1 Inhibitors

The checkpoint kinase Wee1 catalyzes an inhibitory phosphorylation of both CDK1 (CDC2) and CDK2 on tyrosine 15, thus arresting the cell cycle in response to extrinsically induced DNA damage. Deregulated Wee1 expression or activity is believed to be a hallmark of pathology in several types of cancer. For example, Wee1 is often overexpressed in glioblastomas, malignant melanoma, hepatocellular carcinoma, breast cancer, colon carcinoma, lung carcinoma, and head and neck squamous cell carcinoma. Advanced tumors with an increased level of genomic instability may require functional checkpoints to allow for repair of such lethal DNA damage. As such, the present inventors believe that Wee1 represents an attractive target in advanced tumors where its inhibition is believed to result in irreparable DNA damage. Accordingly, one object of the present disclosure includes administration of an inhibitor of Wee1, in combination with or in conjunction with the CCR8 targeting agent(s), to inhibit or kill cancer cells, such as those expressing tor overexpressing CCR8.

A Wee1 inhibitor may be an antibody, peptide, or small molecule that targets Wee1. Alternatively, a Wee1 inhibitor may reduce or eliminate Wee1 activation by one or more signaling molecules, proteins, or other compounds, or can result in the reduction or elimination of Wee1 activation by all signaling molecules, proteins, or other compounds. The term also includes compounds that decrease or eliminate the activation or deactivation of one or more proteins or cell signaling components by Wee1 (e.g., a Wee1 inhibitor can decrease or eliminate Wee1-dependent inactivation of cyclin and Cdk activity). Wee1 inhibitors also include compounds that inhibit Wee1 expression (e.g., compounds that inhibit Wee1 transcription or translation).

Exemplary Wee1 inhibitors that may be used include AZD-1775 (i.e., adavosertib), and inhibitors such as those described in, e.g., U.S. Pat. Nos. 7,834,019; 7,935,708; 8,288,396; 8,436,004; 8,710,065; 8,716,297; 8,791,125; 8,796,289; 9,051,327; 9,181,239; 9,714,244; 9,718,821; and 9,850,247; U.S. Pub. Nos. U.S. 20100113445 and 20160222459; and Int'l Pub. Nos. WO2002090360, WO2015019037, WO2017013436, WO2017216559, WO2018011569, and WO2018011570.

Further Wee1 inhibitors that nay be used include a pyrazolopyrimidine derivative, a pyridopyrimidine, 4-(2-chlorophenyl)-9-hydroxypyrrolo[3,4-c]carbazole-1,3-(2H, 6H)-dione (CAS No. 622855-37-2), 6-butyl-4-(2-chlorophenyl)-9-hydroxypyrrolo[3,4-c]carbazole-1,3-(2H,6H)-dione (CAS No. 62285550-9), 4-(2-phenyl)-9-hydroxypyrrolo[3,4-c]carbazole-1,3-(2H,6H)-dione (CAS No. 1177150-89-8), and an anti-Wee1 small interfering RNA (siRNA) molecule.

### Exemplary DDRi—PARP Inhibitors

Another exemplary DDRi that may be used is an inhibitor of poly(ADP-ribose) polymerase (“PARP”). Inhibitors of the DNA repair protein PARP, referred to individually and collectively as “PARPi”, have been approved for use in a range of solid tumors, such as breast and ovarian cancer, particularly in patients having BRCA1/2 mutations. BRCA1 and 2 function in homologous recombination repair (HRR). When mutated, they induce genomic instability by shifting the DNA repair process from conservative and precise HRR to non-fidelitous methods such as DNA endjoining, which can produce mutations via deletions and insertions.

PARPi have been shown to exhibit synthetic lethality, as exhibited by potent single agent activity, in BRCA1/2 mutant cells. This essentially blocks repair of single-strand DNA breaks. Since HRR is not functional in these tumor cells, cell death results. Because most tumors do not carry BRCA1 or BRCA2 mutations, the potency of PARPi in such tumors is far less pronounced.

To date, the FDA has approved four PARPi drugs (olaparib, niraparib, rucaparib and talazoparib) as monotherapy agents, specifically in patients with germline and somatic mutations in the BRCA1 and BRCA2 genes. Along with veliparib, olaparib, niraparib and rucaparib were among the first generation of PARPi that entered clinical trials. Their IC50 values were found to be in the nanomolar range. In contrast, second generation PARPi like talazoparib have IC50 values in the picomolar range.

These PARPi all bind to the binding site of the cofactor, b nicotinamide adenine dinucleotide (b-NAD+), in the catalytic domain of PARP1 and PARP2. The PARP family of enzymes use NAD+ to covalently add Poly(ADP-ribose) (PAR) chains onto target proteins, a process termed “PARylation.” PARP1 (which is the best-studied member) and PARP2, are important components of the DNA damage response (DDR) pathway. PARP1 is involved in the repair of single-stranded DNA breaks, and possibly other DNA lesions (Woodhouse, et al.; Krishnakumar, et al.). Through its zinc finger domains, PARP1 binds to damaged DNA and then PARylates a series of DNA repair effector proteins, releasing nicotinamide as a by-product (Krishnakumar, et al.). Subsequently, PARP1 auto-PARylation leads to release of the protein from the DNA. The available PARPi, however, differ in their capability to trap PARP1 on DNA, which seems to correlate with cytotoxicity and drug efficacy. Specifically, drugs like talazoparib and olaparib are more effective in trapping PARP1 than are veliparib (Murai, et al., 2012; Murai, et al., 2014).

The efficacy of PARPi in ovarian cancer and breast cancer patients who have loss-of-function mutations in BRCA1 or BRCA2 genes is largely attributed to the genetic concept of synthetic lethality: that proteins of BRCA 1 and 2 normally maintain the integrity of the genome by mediating a DNA repair process, known as homologous recombination repair (HRR); and PARPi causes a persistent DNA lesion that, normally, would otherwise be repaired by HR. In the presence of PARPi, PARP1 is trapped on DNA which stalls progression of the replication fork. This stalling is cytotoxic unless timely repaired by the HR system. In cells lacking effective HR, they are unable to effectively repair these DNA lesions, and thus die.

Again, mutations in BRCA genes and others in the HRR system are not prevalent in many cancer types. So, to better harness the therapeutic benefits of PARPi in such cancers, one can induce “artificial” synthetic lethality by pairing a PARPi with either chemotherapy or radiation therapy. Preclinical studies have demonstrated that combining radiation therapy and PARPi can increase the sensitivity of BRCA1/2 mutant tumor cells to PARP inhibition and extend the sensitivity of non-mutant BRCA tumors to PARP inhibition. Additional studies have shown that ionizing radiation (TR) itself can mediate PARPi synthetic lethality in tumor cells.

Accordingly, the presently disclosed methods include administration of the radioconjugated CCR8 targeting agent agents that deliver ionizing radiation in combination with a PARPi.

In the various embodiments that include a PARPi, the PARPi may, for example, be any known agent performing that function, for example, one approved by the FDA such as olaparib (Lynparza®), niraparib (Zejula®), rucaparib (Rubraca®) and/or talazoparib (Talzenna®). The present inventors realized that the effect of the PARPi may be improved through increases in dsDNA breaks induced by ionizing radiation provided by the radioconjugated CCR8 targeting agent while these repair pathways are being blocked by the PARPi.

### F. CD47 Blockades

The additional agent(s) administered with the radioconjugated CCR8 targeting agent may include one or more CD47 blockades, such as any agent that interferes with, or reduces the activity and/or signaling between CD47 (e.g., on a target cell) and SIRPα (e.g., on a phagocytic cell) through interaction with either CD47 or SIRPα. Non-limiting examples of suitable CD47 blockades include CD47 and/or SIRPα reagents, including without limitation SIRPα polypeptides, anti-SIRPα antibodies, soluble CD47 polypeptides, and anti-CD47 antibodies or antibody fragments.

As used herein, the term “CD47 blockade” refers to any agent that reduces the binding of CD47 (e.g., on a target cell) to SIRPα (e.g., on a phagocytic cell) or otherwise downregulates the “don't eat me” signal of the CD47-. SIRPα pathway. Non-limiting examples of suitable anti-CD47 blockades include SIRPα reagents, including without limitation SIRPα polypeptides, anti-SIRPα antibodies, soluble CD47 polypeptides, and anti-CD47 antibodies or antibody fragments. According to certain aspects, a suitable anti-CD47 agent (e.g. an anti-CD47 antibody, a SIRPα reagent, etc.) specifically binds CD47 to reduce the binding of CD47 to SIRPα.

A CD47 blockade agent for use in the methods of the invention may, for example, up-regulate phagocytosis by at least 10% (e.g., at least 20%, at least 30%, at least 40%, at least 50%, at least 60%, at least 70%, at least 80%, at least 90%, at least 100%, at least 120%, at least 140%, at least 160%, at least 180%, or at least 200%) compared to phagocytosis in the absence of the agent. Similarly, an in vitro assay for levels of tyrosine phosphorylation of SIRPα may, for example, show a decrease in phosphorylation by at least 5% (e.g., at least 10%, at least 15%, at least 20%, at least 30%, at least 40%, at least 50%, at least 60%, at least 70%, at least 80%, at least 90%, or 100%) compared to phosphorylation observed in absence of the agent.

According to certain aspects, a SIRPα reagent may include the portion of SIRPα that is sufficient to bind CD47 at a recognizable affinity, which normally lies between the signal sequence and the transmembrane domain, or a fragment thereof that retains the binding activity. Accordingly, suitable CD47 blockades that may be employed include any of the SIRPα-IgG Fc fusion proteins and others disclosed in U.S. Pat. No. 9,969,789 including without limitation the SIRPα-IgG Fc fusion proteins TTI-621 and TTI-622 (Trillium Therapeutics, Inc.), both of which preferentially bind CD47 on tumor cells while also engaging activating Fc receptors. A SIRPα-IgG Fc fusion protein including the amino acid sequence SEQ ID NO:294, SEQ ID NO:295, or SEQ ID NO:296 may, for example, be used. Still other SIRPα Fe domain fusions proteins that may be used include ALX148 from Alx Oncology or any of those disclosed in Int'l Pub. No WO2017027422 or U.S. Pat. No. 10,696,730.

According to certain aspects, an anti-CD47 agent includes an antibody that specifically binds CD47 (i.e., an anti-CD47 antibody) and reduces the interaction between CD47 on one cell (e.g., an infected cell) and SIRPα on another cell (e.g., a phagocytic cell). Non-limiting examples of suitable antibodies include clones B6H12, 5F9, 8B6, and C3 (for example as described in International Pub. No. WO 2011/143624). Suitable anti-CD47 antibodies include fully human, humanized or chimeric versions of such antibodies.

Exemplary human or humanized antibodies useful for in vivo applications in humans due to their low antigenicity include at least monoclonal antibodies against CD47, such as Hu5F9-G4, a humanized monoclonal antibody available from Gilead as Magrolimab (Sikic, et al. (2019) Journal of Clinical Oncology 37:946); Lemzoparlimab and TJC4 from I-Mab Biopharma; AO-176 from Arch Oncology, Inc; AK 117 from Akesobio Australia Pty; IMC-002 from Innovent Biologics; ZL-1201 from Zia Lab; SHR-1603 from Jiangsu HengRui Medicine Co.; and SRF231 from Surface Oncology. Bispecific monoclonal antibodies are also available, such as IBI-322, targeting both CD47 and PD-L1 from Innovent Biologics.

AO-176, in addition to inducing tumor phagocytosis through blocking the CD47-SIRPα interaction, has been found to preferentially bind tumor cells versus normal cells (particularly RBCs where binding is negligible) and directly kills tumor versus normal cells.

Antibodies against SIRPα may also be used as CD47 blockades. Without limitation, anti-SIRPα antibodies (also referred to as SIRPα antibodies herein) that may be used in or embodied in any of the aspects of the invention include but are not limited to the following anti-SIRPα antibodies, antibodies that include one or both of the heavy chain and light chain variable regions of the following anti-SIRPα antibodies, antibodies that include one or both of the heavy chain and the light chain CDRs of any of the following anti-SIRPα antibodies, and antigen-binding fragments of any of said anti-SIRPα antibodies:
(1) ADU-1805 (Sairopa B. V.; Aduro) and any of the SIRPα antibodies disclosed in Intl. Pub. No. WO2018190719 or U.S. Pat. No. 10,851,164;
(2) AL008 (Alector LLC) and any of the SIRPα antibodies disclosed in Intl. Pub. No. WO2018107058, U.S. Pub. No. 20190275150, or U.S. Pub. No. 20210179728;
(3) AL008 (Apexigen, Inc.) and any of the SIRPα antibodies disclosed in Intl. Pub. No. WO2021174127 or U.S. App. No. 63/108,547;
(4) SIRP-1 and SIRP-2 (Arch Oncology, Inc.) and any of the SIRPα antibodies disclosed in Intl. Pub. No. WO2021222746, U.S. App. No. 63/107,200 or U.S. Pub. No. 20200297842;
(5) OSE-172 (a/k/a BI 765063; Boehringer Ingelheim) and any of the SIRPα antibodies disclosed in Intl. Pub. No. WO2017178653 or U.S. Pub. No. 20190127477;
(6) CC-95251 (Bristol Myers Squibb; Celgene) and any of the SIRPα antibodies disclosed in Intl. Pub. No. WO2020068752 or U.S. Pub. No. 20200102387;
(7) ES004 (Elpiscience Biopharma) and any of the SIRPα antibodies disclosed in Intl. Pub. No. WO2021032078 or U.S. Pub. No. 20210347908;
(8) FSI-189 (Gilead Sciences, Inc.; Forty Seven) and any of the SIRPα antibodies disclosed in Intl. Pub. No. WO2019023347, U.S. Pat. No. 10,961,318 or U.S. Pub. No. 20210171654;
(9) BYON4228 (Byondis B. V.; Synthon) and any of the SIRPα antibodies disclosed in Intl. Pub. No. WO2018210793, Intl. Pub. No. WO2018210795, or U.S. Pub. No. 20210070874;
(10) any of the SIRPα antibodies disclosed in Intl. Pub. No. WO2018057669, U.S. Pat. No. 11,242,404 or U.S. Pub. No. 20220002434 (Alexo Therapeutics Inc., now ALX Oncology Inc.);
(11) any of the SIRPα antibodies disclosed in Intl. Pub. No. WO2015138600, U.S. Pat. No. 10,781,256 or U.S. Pat. No. 10,081,680 (Leland Stanford Junior University);
(12) BR105 (Bioray Pharma); or
(13) BSI-050 (Biosion, Inc.).

The CD47 blockade may alternatively, or additionally, include agents that modulate the expression of CD47 and/or SIRPα, such as phosphorodiamidate morpholino oligomers (PMO) that block translation of CD47 such as MBT-001 (PMO, morpholino, Sequence: 5′-CGTCACAGGCAGGACCCACTGCCCA-3′) [SEQ ID NO:292]) or any of the PMO oligomer CD47 inhibitors disclosed in any of U.S. Pat. Nos. 8,557,788, 8,236,313, 10,370,439 and Int'l Pub. No. WO2008060785.

Small molecule inhibitors of the CD47-SIRPα axis may also be used, such as RRx-001 (1-bromoacetyl-3,3 dinitroazetidine) from EpicentRx and Azelnidipine (CAS number 123524-52-7), or pharmaceutically acceptable salts thereof. Such small molecule CD47 blockades may, for example, be administered at a dose of 5-100 mg/m², 5-50 mg/m², 5-25 mg/m², 10-25 mg/m², or 10-20 mg/m², or in any of the dose ranges or at any of the doses described herein. Administration of RRx-001 may, for example, be once or twice weekly and be by intravenous infusion. The duration of administration may, for example, be at least four weeks.

Various CD47 blockades that may be used are found in Table 1 of Zhang, et al., (2020), Frontiers in Immunology vol 11, article 18, and in Table 2 below.

TABLE 2

| Company | Approach | Agent/Program |
|---|---|---|
| Akesobio Australia | CD47 mAb | AK117 |
| Pty Ltd | | |
| Arch Oncology (Tioma | CD47 mAb | AO-176 |
| Therapeutics) | | |
| Elpiscience Biopharma | CD47 | ES004 |
| Inc. | | |
| EpicentRx | Small molecule | RRx-001 |
| | inhibitor of | (1-bromoacetyl- 3,3 |
| | dinitroazetidine | dinitroazetidine) |
| | hypoxia sensor to | |
| | downregulate | |
| | CD47/SIRPα | |
| ImmuneOncia | CD47 mAb human | IMC-002 |
| Therapeutics | | |
| Innovent Biologics | CD47 mAb | IBI-188 (CD47 mAb) |
| | CD47/PD-L1 | IBI-322 (Bispecific) |
| | bispecific mAb | |
| OSE | SIRPα mAb | BI 765063 (OSE-172) |
| Zai Lab | CD47 mAb | ZL-1201 |
| Alx Oncology | High-affinity | ALX148 |
| | SIRPα-Fc | |
| Gilead/Forty Seven | CD47 mAb | Magrolimab |
| | SIRPα mAb | FSI-189 |
| I-Mab Biopharma | CD47 mAb | TJC4 |
| Jiangsu HengRui | CD47 mAb | SHR-1603 |
| Medicine Co., Ltd. | | |
| Surface Oncology | CD47 mAb human | SRF231 |
| Morphiex | CD47 targeting | MBT-001 |
| | phosphorodiamidate | |
| | morpholino oligomers | |

Therapeutically effective doses of an anti-CD47 antibody or other protein CD47 blockade may, for example, be a dose that leads to sustained serum levels of the protein of about 40 μg/ml or more (e.g., about 50 ug/ml or more, about 60 ug/ml or more, about 75 ug/ml or more, about 100 ug/ml or more, about 125 ug/ml or more, or about 150 ug/ml or more). Therapeutically effective doses or administration of a CD47 blockade, such as an anti-CD47 antibody or SIRPα fusion protein or small molecule, include, for example, amounts of 0.05-10 mg/kg (agent weight/subject weight), such as at least 0.1 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg, 7.0 mg/kg, 7.5 mg/kg, 8.0 mg/kg, 8.5 mg/kg, 9.0 mg/kg; or not more than 10 mg/kg, 9.5 mg/kg, 9.0 mg/kg, 8.5 mg/kg, 8.0 mg/kg, 7.5 mg/kg, 7.0 mg/kg, 6.5 mg/kg, 6.0 mg/kg, 5.5 mg/kg, 5.0 mg/kg, 4.5 mg/kg, 4.0 mg/kg, 3.5 mg/kg, 3.0 mg/kg, 2.5 mg/kg, 2.0 mg/kg, 1.5 mg/kg, 1.0 mg/kg, or any combination of these upper and lower limits. Therapeutically effective doses of a small molecule CD47 blockade such as those disclosed herein also, for example, include 0.01 mg/kg to 1,000 mg/kg and any subrange or value of mg/kg therein such as 0.01 mg/kg to 500 mg/kg or 0.05 mg/kg to 500 mg/kg, or 0.5 mg/kg to 200 mg/kg, or 0.5 mg/kg to 150 mg/kg, or 1.0 mg/kg to 100 mg/kg, or 10 mg/kg to 50 mg/kg.

According to certain aspects, the anti-CD47 agent is a soluble CD47 polypeptide that specifically binds SIRPα and reduces the interaction between CD47 on one cell (e.g., an infected cell) and SIRPα on another cell (e.g., a phagocytic cell). A suitable soluble CD47 polypeptide can bind SIRPα without activating or stimulating signaling through SIRPα because activation of SIRPα would inhibit phagocytosis. Instead, suitable soluble CD47 polypeptides facilitate the preferential phagocytosis of infected cells over non-infected cells. Those cells that express higher levels of CD47 (e.g., infected cells) relative to normal, non-target cells (normal cells) will be preferentially phagocytosed. Thus, a suitable soluble CD47 polypeptide specifically binds SIRPα without activating/stimulating enough of a signaling response to inhibit phagocytosis. In some cases, a suitable soluble CD47 polypeptide can be a fusion protein (for example, as described in U.S. Pub. No. 20100239579).

### G. Adoptive Cell Therapy

The additional therapy or modality administered with the radioconjugated CCR8 targeting agent may include an adoptive cell therapy (ACT). ACT is the transfer of ex vivo grown and/or modified cells, most commonly immune-derived cells, into a host with the goal of transferring the immunologic functionality and characteristics of the transplanted cells.

Current ACTs may include genetically modifying immune cells with target antigens through the expression of chimeric antigen receptors (CARs). For example, CAR cell therapy can involve genetically modifying autologous or allogeneic immune cells to express chimeric antigen receptors (CARs) that target tumor cell antigens, and typically employ the single chain fraction variable region of a monoclonal antibody designed to recognize a cell surface antigen in a human leukocyte antigen-independent manner.

The ACT may also include recombinant T-cell receptor (TCR) therapy. TCRs on lymphocytes can recognize tumor-specific proteins typically found on the inside of cells. They do so by specifically recognizing processed peptides (derived from those proteins) that are complexed to major histocompatibility (MHC) antigens. In TCR CAR cell therapy, a TCR is selected for specific recognition of a tumor-expressed neoantigen and engineered for expression on a patient's T-cells. In some cases, the TCR or the CAR may be directed to the endogenous TCR locus. For example, the TRAC locus (T-cell receptor gene) may be targeted via gene editing (e.g., CRISPR/cas9 technology, TALEN, or ZFN), effectively replacing the endogenous TCR with the recombinant TCR gene.

In addition to autologous cells, allogeneic donor lymphocytes may also be used for generating CARs or TCRs. In this case, the endogenous TCR on the donor cells must be deleted to reduce the potential for graft-versus-host disease. Gene editing technologies are an effective way to introduce mutations to silence or ablate the endogenous TCR.

Finally, ACT methods can further include administering tumor-infiltrating lymphocytes (TTLs), which may be used to treat patients with advanced solid tumors such as melanoma and hematologic malignancies

These ACTs provide a method of promoting regression of a cancer in a subject, and may include (i) collecting immune cells (leukapheresis); (ii) expanding the immune cells (culturing); and (iii) administering to the subject the expanded immune cells.

The ACT may use autologous cells, e.g., isolated by leukapheresis, transduced and selected approximately 4 weeks immediately prior to administration, as is common in adoptive T-cell therapies, or allogeneic cells as typical for treatment of infections or graft-versus-host disease.

Moreover, the ACT may be xenogeneic. The ACT may also include transfer of allogeneic lymphocytes isolated, prepared, and stored (e.g., frozen) “off-the-shelf” from a healthy donor which may be used to treat patients with advanced solid tumors such as melanoma and hematologic malignancies.

The ACT may, for example, use cell types such as T-cells, natural killer (NK) cells, delta-gamma T-cells, regulatory T-cells, dendritic cells, and peripheral blood mononuclear cells. The ACT may use monocytes with the purpose of inducing differentiation to dendritic cells subsequent to contact with tumor antigens. Given that monocytes have a fixed mitotic index, permanent manipulation of the host may be diminished.

The ACT may, for example, include a population of cells (e.g., T cells, NK cells, or dendritic cells) expressing a CAR or TCR (referred herein simply as “CAR cell therapy”). The CAR cell therapy generally involves engineering a T cell, NK cell, or dendritic cell to target a tumor antigen of interest by way of engineering a desired antigen binding domain that specifically binds to an antigen on a tumor cell. In the context of the present invention, “tumor antigen” or “proliferative disorder antigen” or “antigen associated with a proliferative disorder,” refers to antigens that are common to specific proliferative disorders such as cancer. The antigens discussed herein are merely included by way of example and are not intended to be exclusive, and further examples will be readily apparent to those of skill in the art.

A number of tumor-specific and tumor-associated antigens have been catalogued and are maintained in databases, such as the Database of Collected Peptides for Neoantigen (biostatistics.online/dbPepNeo/), Tumor-Specific NeoAntigen database (biopharm.zju.edu.cn/tsnadb/), TANTIGEN 2.0: Tumor T-cell Antigen Database (projects.met-hilab.org/tadb/), and Cancer Antigenic Peptide Database (caped.icp.ucl.ac.be/). Tumor antigens listed in these databases, as well as additional antigens identified in patients, may for example be used as ACT targets in aspects of the present invention that include ACT.

In aspects of the invention involving CAR/TCR expressing cell ACT, exemplary antigen targets recognized by the CAR/TCR may include CD19, CD20, CD22, CD30, CD33, CD38, CD123, CD138, CS-1, B-cell maturation antigen (BCMA), MAGEA3, MAGEA3/A6, KRAS, CLL1, MUC-1, HER2, EpCam, GD2, GPA7, PSCA, EGFR, EGFRvIII, ROR1, mesothelin, CD33/IL3Ra, c-Met, CD37, PSMA, Glycolipid F77, GD-2, gp100, NY-ESO-1 TCR, FRalpha, GUCY2C, CD24, CD44, CD133, CD166, CA-125, HE4, Oval, estrogen receptor, progesterone receptor, uPA, PAI-1, MICA, MICB, ULBP1, ULBP2, ULBP3, ULBP4, ULBP5 or ULBP6, or any combination thereof (e.g., both CD33 and CD123).

The engineered cell of the CAR cell therapy may, for example, include an antigen binding domain capable of targeting two or more different antigens (i.e., bispecific or bivalent, trispecific or trivalent, tetraspecific, etc.). For example, the CAR cell therapy may include a first antigen binding domain that binds to a first antigen and a second antigen binding domain that binds to a second antigen (e.g., tandem CAR). Alternatively, each cell in the population of cells, or the overall population of cells, may include more than one distinct CAR T cell or NK cell (e.g., construct), wherein each CAR T cell or NK cell construct may recognize a different antigen. For example, the population of CAR T cells may target three antigens such as, for example, HER2, IL13Rα2, and EphA2.

The population of cells, whether autologous or allogeneic, may, for example, be engineered using gene editing technology such as by CRISPR/cas9 (clustered regularly interspaced short palindromic repeats/CRISPR associated protein 9), Zinc Finger Nucleases (ZFN), or transcription activator-like effector nuclease (TALEN). These technologies, recognized and practiced in the art of genetic engineering, enable selective editing, disruption, or insertion of targeted sequences to modify the genome of the cell of interest. Accordingly, isolated autologous or allogeneic cells for adoptive transfer practiced in the current invention may be edited to delete or replace a known gene or sequence. For example, the T cell receptor (TCR) in an allogeneic T cell population may be deleted or replaced prior to or after CAR-T transduction as a means to eliminate graft-versus-host disease in recipient patients.

CAR cell therapy has shown unprecedented initial response rates in advanced B-cell malignancies; however, relapse after CAR cell infusion, and limited therapeutic success in solid tumors is a major hurdle in successful CAR regimens. This latter limitation is mainly attributable to the hostile microenvironment of a solid tumor. Anatomical barriers such as the tumor stroma, and immunosuppressive cytokines and immune cells which are harmful to the infiltration of infused CAR modified cells into tumor sites, both limit the success of CAR cell therapy. Accordingly, methods of the present invention that deplete or abolish CCR8 expressing Tregs from the tumor microenvironment may lead to enhanced effector T cell and/or NK cell function and enhance the efficacy of an ACT such as a CAR cell therapy.

In one aspect of the present invention, autologous cells (e.g., T-cell or NK-cells or dendritic cells) may be collected from a subject. These cells may be obtained from a number of sources, including peripheral blood mononuclear cells, bone marrow, lymph node tissue, cord blood, thymus tissue, tissue from a site of infection, ascites, pleural effusion, spleen tissue, and tumors. Alternatively, or additionally, allogeneic or xenogeneic cells may be used, typically isolated from healthy donors. When the T-cells, NK cells, dendritic cells, or pluripotent stem cells are allogeneic or xenogeneic cells, any number of cell lines available in the art may be used.

The cells may be obtained from a unit of blood collected from a subject using any number of techniques known to the skilled artisan, such as Ficoll™ separation. Cells from the circulating blood of an individual may be obtained by apheresis. The apheresis product typically contains lymphocytes, including T-cells, monocytes, granulocytes, B-cells, other nucleated white blood cells, red blood cells, and platelets. In some cases, the collection of blood samples or apheresis product from a subject may be at any time period prior to when the expanded cells as described herein might be needed. As such, the source of the cells to be engineered and/or expanded can be collected at any time point necessary, and desired cells, such as T-cells, NK-cells, dendritic cells, or TILs, can be isolated and frozen for later use in ACT.

The blood sample or apheresis may be from a generally healthy subject, or a generally healthy subject who is at risk of developing a disease, but who has not yet developed a disease, and the cells of interest may be isolated and frozen for later use. The cells may be expanded, frozen, and used at a later time. In certain cases, the cell samples may be collected from a subject shortly after diagnosis of a particular disease as described herein but prior to any treatments.

Enrichment of a cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD1 1b, CD16, HLA-DR, and CD8. According to certain aspects of the present invention, it may be desirable to enrich for or positively select for a cell population. For example, positive enrichment for a regulatory T-cell may use positive selection for CD4+, CD25+, CD62Lhi, GITR+, and FoxP3+.

The collected cells may, for example, be engineered to express the CAR by any of the methods known in the art. Moreover, the engineered cells may be expanded by any of the methods known in the art.

The population of cells expressing the CAR or TCR may, for example, be administered to the subject by dose fractionation, wherein a first percentage of a total dose is administered on a first day of treatment, a second percentage of the total dose is administered on a subsequent day of treatment, and optionally, a third percentage of the total dose is administered on a yet subsequent day of treatment.

An exemplary total dose includes 10³ to 10¹¹ cells/kg body weight of the subject, such as 10³ to 10¹⁰ cells/kg body weight, or 10³ to 10⁹ cells/kg body weight of the subject, or 10³ to 10⁸ cells/kg body weight of the subject, or 10³ to 10⁷ cells/kg body weight of the subject, or 10³ to 10⁶ cells/kg body weight of the subject, or 10³ to 10⁵ cells/kg body weight of the subject. Moreover, an exemplary total dose includes 10⁴ to 10¹¹ cells/kg body weight of the subject, such as 10⁵ to 10¹¹ cells/kg body weight, or 10⁶ to 10¹¹ cells/kg body weight of the subject, or 10⁷ to 10¹¹ cells/kg body weight of the subject.

An exemplary total dose may be administered based on a patient body surface area rather than the body weight. As such, the total dose may include 10³ to 10¹³ cells per m².

An exemplary dose may be based on a flat or fixed dosing schedule rather than on body weight or body surface area. Flat-fixed dosing may avoid potential dose calculation mistakes. Additionally, genotyping and phenotyping strategies, and therapeutic drug monitoring, may be used to calculate the proper dose. That is, dosing may be based on a patient's immune repertoire of immunosuppressive cells (e.g., T-regs, MDSC), and/or disease burden. As such, the total dose may include 10³ to 10¹³ total cells.

Cells useful in the ACT may, for example, be obtained from a subject directly following a treatment. In this regard, it has been observed that following certain cancer treatments, in particular treatments with drugs that damage the immune system, shortly after treatment during the period when subjects would normally be recovering from the treatment, the quality of certain cells (e.g., T-cells) obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T-cells, NK-cells, dendritic cells, or other cells of the hematopoietic lineage, during this recovery phase.

The radioconjugated CCR8 targeting agent may, for example, be administered after administration of the effective dose of the ACT or CAR cell therapy. The effective amount of the radioconjugated CCR8 targeting agent may be an amount sufficient to induce depletion or ablation of CCR8 expressing Tregs in the subject.

The radioconjugated CCR8 targeting agent may, for example, be administered to the subject before administration of the effective dose of the ACT or CAR cell therapy. That is, the method may include an apheresis step to collect a population of cells that may engineered to express a CAR, and expanded, followed by administration of a first dose of an effective amount of a radioconjugated CCR8 targeting agent. The population of cells expressing the CAR may then be administered to the subject.

The radioconjugated CCR8 targeting agent may, for example, be administered to the subject both before and after administration of the effective dose of the ACT or CAR cell therapy, or the effective dose of the ACT or CAR cell therapy may be administered both before and after the radioconjugated CCR8 targeting agent. A second dose of either therapy (radioconjugated CCR8 targeting agent or ACT or CAR cell therapy) may be the same or different from a first dose, in either effective amount and/or target (e.g., different epitope of radioconjugated CCR8 antibody, different antigen target for the ACT or CAR cell therapy, different cell types of the CAR, different cytoplasmic co-stimulatory signaling domains of the CAR, etc.).

Administration of a second dose of either the radioconjugated CCR8 targeting agent or a population of cells expressing the CAR (or TCR) may be useful in subjects who have not shown a complete response (CR) or who has relapsed or is identified as having relapsed, such as shown by tumor size and/or increase in the population of CCR8-positive Tregs in the tumor microenvironment.

Accordingly, the present invention provides methods for the treatment of a proliferative disease, such as a solid cancer, which include administration of a radioconjugated CCR8 targeting agent and an adoptive cell therapy. The adoptive cell therapy may, for example, include apheresis of autologous cells which may be gene edited prior to reinfusion (adoptive cell therapy such as CAR T-cell therapy), such as after depletion of Tregs by treatment with the radioconjugated CCR8 targeting agent, or simultaneous with administration of radioconjugated CCR8 targeting agent.

The radioconjugated CCR8 targeting agent may, for example, be provided as a single dose 3 to 9 days, such as 6 to 8 days, prior to the adoptive cell therapy to affect depletion or ablation of CCR8 positive Treg cells in the tumor microenvironment.

Thus, the present invention provides a method for treating a subject afflicted with cancer, such as a solid tumor, including (i) administering to the subject an amount of a radioconjugated CCR8 targeting agent effective to deplete the subject's CCR8 positive Tregs, and (ii) either before, simultaneous or overlapping with, or after (such as after a suitable time period), performing adoptive cell therapy on the subject to treat the subject's cancer. Preferably, the subject is human.

Although the invention provides certain aspects that involve cell therapy and/or the administration of cells therefor to a subject, also provided are aspects of the invention that do not involve cell therapy or the administration of cells to the subject, such as do not involve the administration of genetically edited cells to the subject, and/or do not involve the administration of CAR-T or recombinant TCR cells to the subject.

### EXAMPLES

### Example 1: Production of Radiolabeled CCR8 Targeting Agent

The CCR8 targeting agent, such as a monoclonal antibody against CCR8 or an antigen-binding fragment thereof, may be labeled with a metallic radionuclide (radiometal) such as ⁶⁷Ga, ⁶⁸Ga, ^{99m}Tc, ¹¹¹In, ^{114m}In, ¹⁷⁷Lu, ⁶⁴Cu, ⁴⁴Sc, ⁴⁷Sc, ⁸⁶Y, ⁹⁰Y, ⁸⁹Zr, ²¹²Bi, ²¹³Bi, ²¹²Pb, ²²⁵Ac, ¹⁸⁶Re or ¹⁸⁸Re. Radionuclides that may be used for diagnostic purposes include but are not limited to ⁶⁷Ga, ^{99m}Tc, ¹¹¹In, and ¹⁷⁷Lu, which are useful in single photon emission computed tomography (SPECT), and ⁶⁸Ga, ⁶⁴Cu, ⁴⁴Sc, ⁸⁶Y, and ⁸⁹Zr, which are useful in positron emission tomography (PET). Radionuclides that may be used for therapeutic purposes include but are not limited to ⁴⁷Sc, ^{114m}In, ¹⁷⁷Lu, ⁹⁰Y, ²¹²Bi, ²¹³Bi, ²¹²Pb, ²²⁵Ac, ¹⁸⁶Re, and ¹⁸⁸Re. Int'l Pub. No. U.S.

The CCR8 targeting agent, such as anti-CCR8 monoclonal antibody, and optionally other targeting agents may, for example, be labeled with Iodine-131 (¹³¹I) or other Iodine isotopes according to the radio-iodination procedures detailed in International Pub. No. WO 2017155937 and U.S. Pat. No. 10,420,851 or with Actinium-225 (²²⁵Ac) or Lutetium-177 (¹⁷⁷Lu), each of which can be chelated by DOTA, according to procedures described in U.S. Pat. No. 9,603,954.

Preparing the DOTA-conjugated antibody using p-SCN-Bn-DOTA: antibody conjugates may be prepared by reacting a concentrated solution of monoclonal anti-CCR8 antibody with p-SCN-Bn-DOTA in bicarbonate or in phosphate buffers at pH between about 8 and about 9 and by incubation at either about 37° C. or at room temperature. The conjugates may be purified from excess of the bifunctional chelator by repeated filtration or centrifugation and by gravity size exclusion chromatography (SEC). During the purification process, the bicarbonate or phosphate buffer is changed to N-2-Hydroxyethylpiperazine-N-2-ethanesulfonic acid (HEPES; Free Acid) or acetate medium. Conjugates may be characterized by size exclusion high performance liquid chromatography (SE-HPLC).

Preparing the DOTA-conjugated antibody using PODS-DOTA: DOTA may be conjugated to a monoclonal antibody, such as an IgG, or an antigen-binding portion thereof, using PODS-DOTA in the presence of TCEP, a mild reducing agent that cleaves the inter-chain disulfide bonds within an immunoglobin according to the methods set forth in U.S. Pat. No. 11,000,604. The structure of PODS-DOTA is

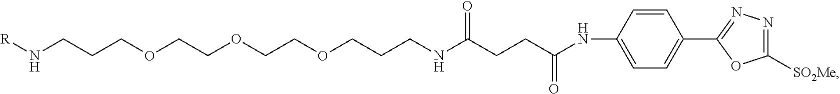

wherein R is a covalently bound DOTA moiety.

In more detail, to a suspension of 200 μg of antibody in PBS pH 7.4 (1 mg/mL) 1.33 μL of a fresh TCEP solution (10 mM in water, 10 eq.) is added and the appropriate volume of a solution of PODS-DOTA (1 mM in DMSO). The reaction mixture is then stirred on a thermomixer (25° C. or 37° C.) for 30 min, 2 h, or 24 h. The conjugate is then purified on a size exclusion column (Sephadex G-25 M, PD-10 column, GE Healthcare; dead volume=2.5 mL, eluted with 2 mL of PBS, pH 7.4) and concentrated using centrifugal filtration units with a 50,000 Da molecular weight cut off (AMICON™ Ultra 4 Centrifugal Filtration Units, Millipore Corp. Billerica, Mass.)

Radiolabeling the DOTA-conjugated antibody: The CCR8 targeting agent, such as antibody, may be conjugated to a chelator-bearing linker, such as any of the linkers described in the above indicated patent documents or as exemplified above. An exemplary linker includes at least dodecane tetraacetic acid (DOTA) or a derivative thereof, wherein a goal of the conjugation reaction is to achieve a DOTA-antibody ratio of 3:1 to 5:1. Chelation with the radionuclide (e.g., ¹⁷⁷Lu or ²²⁵Ac) may then be performed and efficiency and purity of the resulting radioconjugated anti-CCR8 antibody may be determined by HPLC and iTLC.

An exemplary labeling reaction for ²²⁵Ac is as follows: A reaction including 15 μl 0.15M NH₄OAc buffer, pH=6.5 and 2 L (10 g) DOTA-anti-CCR8 (5 mg/ml) may be mixed in an Eppendorf reaction tube, and 4 L ²²⁵Ac (10 μCi) in 0.05 M HCl subsequently added. The contents of the tube may be mixed with a pipette tip and the reaction mixture incubated at 37° C. for 90 minutes with shaking at 100 rpm. At the end of the incubation period, 3 μL of a 1 mM DTPA solution may be added to the reaction mixture and incubated at room temperature for 20 minutes to bind the unreacted ²²⁵Ac into the ²²⁵Ac-DTPA complex. Instant thin layer chromatography with 10 cm silica gel strip and 10 mM EDTA/normal saline mobile phase may be used to determine the radiochemical purity of ²²⁵Ac-DOTA-anti-CCR8 through separating ²²⁵Ac-labeled anti-CCR8 (²²⁵Ac-DOTA-anti-CCR8) from free ²²⁵Ac (²²⁵Ac-DTPA). In this system, the radiolabeled antibody stays at the point of application and ²²⁵Ac-DTPA moves with the solvent front. The strips may be cut in halves and counted in the gamma counter equipped with the multichannel analyzer using channels 72-110 for ²²⁵Ac to exclude its daughters.

Purification: An exemplary radioconjugated CCR8 targeting agent, such as 225Ac-DOTA-anti-CCR8, may be purified either on PD10 columns pre-blocked with 1% HSA or on Vivaspin centrifugal concentrators with a 50 kDa MW cut-off with 2×1.5 mL washes, 3 minutes per spin. HPLC analyses of the ²²⁵Ac-DOTA-anti-CCR8 after purification may be conducted using a Waters HPLC system equipped with flow-through Waters UV and Bioscan Radiation detectors, using a TSK3000SW XL column eluted with PBS at pH=7.4 and a flow rate of 1 ml/min.

Stability determination: An exemplary radioconjugated CCR8 targeting agent, such as ²²⁵Ac-DOTA-anti-CCR8, may be used for stability determination, wherein the ²²⁵Ac-DOTA-anti-CCR8 may be tested either in the original volume or diluted (2-10 fold) with the working buffer (0.15 M NH₄OAc) and incubated at room temperature (rt) for 48 hours or at 4° C. for 96 hours and tested by ITLC. Stability is determined by comparison of the intact radiolabeled anti-CCR8 before and after incubation. Other antibodies labeled with ²²⁵Ac have been found to be stable at 4° C. for up to 96 hrs.

Immunoreactivity (IR) determination: An exemplary radioconjugated CCR8 targeting agent, such as ²²⁵Ac-DOTA-anti-CCR8, may be used in immunoreactivity experiments. CCR8 positive cells and control CCR8 negative cells may be used in the amounts of 1.0-7.5 million cells per sample to investigate the amount of binding (percent radioactivity binding to cells after several washes; or using an immunoreactive fraction (IRF) bead assay may be performed according to methods disclosed in as described by Sharma, 2019). Prior assays for other antibodies radiolabeled with ¹¹¹In or ²²⁵Ac demonstrated about 50-60% immunoreactivity.

### Example 2: Methods for Testing the Radioconjugated CCR8 Targeting Agent as a Single Modality: An Example of a Syngeneic Solid Tumor Model Using CT26 Murine Colon Carcinoma Cell Line

### Biodistribution of Radioconjugated Anti-CCR8 mAb:

BALB/c mice will be injected subcutaneously in the right flank with 2×10⁶ CT26 cells. When tumors grow to approximately 100 mm³, mice will be treated intravenously with anti-CCR8 mAb (clone SA214G2) conjugated with ¹¹¹In (Indium-111), a gamma-emitting radioisotope that does not cause DNA damage or cancer cell death. Blood, tumor, and major organs will be harvested 4, 24, 48, 96, and 168 hours post ¹¹¹In-anti-CCR8 administration for dosimetry calculations in which the absorbed dose of radiation is quantified for each tissue and time point. ¹¹¹In-anti-CCR8 should home to tumor tissue but not to healthy tissue because only tumor-infiltrating Tregs express CCR8.

Dose Escalation Study with ²²⁵Ac Anti-CCR8 mAb:

²²⁵Ac causes DNA damage and cancer cell death. BALB/c mice will be injected subcutaneously in the right flank with 2×10⁶ CT26 cells. When tumors grow to approximately 100 mm³, mice will be treated intravenously with various doses of ²²⁵Ac-conjugated anti-CCR8 antibody (0, 100, 200, 400, 500 nCi ²²⁵Ac; 500 ng total amount of IgG; clone SA214G2). This dose escalation study will determine the maximum tolerated dose (MTD) and minimum effective dose (MED). MTD will be defined as the highest dose that permits all treated mice to maintain weight above 85% of the baseline weight, and MED will be defined as the minimum dose that leads to quantifiable shrinkage of the tumors. Total body weights and tumor volumes will be measured twice a week, and a Kaplan-Meier survival curve will be generated. In a separate experiment, the ablation of Tregs in the tumor will be confirmed by immunohistochemistry. After confirmation of single agent efficacy, combination approaches will be tested next.

### Example 3: Antibody Dose—Radioconjugated Versus Unconjugated

Conjugating an antibody with ²²⁵Ac would substantially decrease the amount of total antibody necessary to achieve tumor response. Based on previous experience comparing the efficacy of ²²⁵Ac conjugated- and unconjugated monoclonal antibodies (Dawicki, 2019), the amount of anti-CCR8 antibody required to elicit tumor response may be decreased approximately 30-fold if conjugated with ²²⁵Ac. Furthermore, due to the potency of the alpha-emitter, a single administration of the radioconjugated agent should be sufficient to observe tumor reduction. However, because biological responses to antitumor therapy are difficult to predict, we will also test hypofractionated regimens, where the total radiation dose is divided into two or three administrations to determine which schedule is optimal.

### Example 4—Exemplary PARPi Administration and Dosing Regimes

### (A) Olaparib (Lynparza®)—Normal and Reduced Dosing Regimens

Olaparib is sold by AstraZeneca under the brand name Lynparza®. Lynparza® is sold in tablet form at 100 mg and 150 mg. The dosage is 300 mg taken orally twice daily for a daily total of 600 mg. Dosing continues until disease progression or unacceptable toxicity. This dosing regimen is referred to herein as the “normal” human dosing regimen for Lynparza®, regardless of the disorder treated. Any dosing regimen having a shorter duration (e.g., 21 days) or involving the administration of less Lynparza® (e.g., 300 mg/day) is referred to herein as a “reduced” human dosing regimen. Examples of reduced human dosing regimens include the following: (i) 550 mg/day; (ii) 500 mg/day; (iii) 450 mg/day; (iv) 400 mg/day; (v) 350 mg/day; (vi) 300 mg/day; (vii) 250 mg/day; (viii) 200 mg/day; (ix) 150 mg/day; (x) 100 mg/day; or (xi) 50 mg/day.

### (B) Niraparib (Zejula®)—Normal and Reduced Dosing Regimens

Niraparib is sold by Tesaro under the brand name Zejula®. Zejula® is sold in capsule form at 100 mg. The dosage is 300 mg taken orally once daily. Dosing continues until disease progression or unacceptable adverse reaction. This dosing regimen is referred to herein as the “normal” human dosing regimen for Zejula®, regardless of the disorder treated. Any dosing regimen having a shorter duration (e.g., 21 days) or involving the administration of less Zejula® (e.g., 150 mg/day) is referred to herein as a “reduced” human dosing regimen. Examples of reduced human dosing regimens include the following: (i) 250 mg/day; (ii) 200 mg/day; (iii) 150 mg/day; (iv) 100 mg/day; or (v) 50 mg/day.

### (C) Rucaparib (Rubraca®)—Normal and Reduced Dosing Regimens

Rucaparib is sold by Clovis Oncology, Inc. under the brand name Rubraca™ Rubraca™ is sold in tablet form at 200 mg and 300 mg. The dosage is 600 mg taken orally twice daily for a daily total of 1,200 mg. Dosing continues until disease progression or unacceptable toxicity. This dosing regimen is referred to herein as the “normal” human dosing regimen for Rubraca™, regardless of the disorder treated. Any dosing regimen having a shorter duration (e.g., 21 days) or involving the administration of less Rubraca™ (e.g., 600 mg/day) is referred to herein as a “reduced” human dosing regimen. Examples of reduced human dosing regimens include the following: (i) 1,150 mg/day; (ii) 1,100 mg/day; (iii) 1,050 mg/day; (iv) 1,000 mg/day; (v) 950 mg/day; (vi) 900 mg/day; (vii) 850 mg/day; (viii) 800 mg/day; (ix) 750 mg/day; (x) 700 mg/day; (xi) 650 mg/day; (xii) 600 mg/day; (xiii) 550 mg/day; (xiv) 500 mg/day; (xv) 450 mg/day; (xvi) 400 mg/day; (xvii) 350 mg/day; (xviii) 300 mg/day; (xix) 250 mg/day; (xx) 200 mg/day; (xxi) 150 mg/day; or (xxii) 100 mg/day.

### (D) Talazoparib (Talzenna™)—Normal and Reduced Dosing Regimens

Talazoparib is sold by Pfizer Labs under the brand name Talzenna™. Talzenna™ is sold in capsule form at 1 mg. The dosage is 1 mg taken orally. Dosing continues until disease progression or unacceptable toxicity. This dosing regimen is referred to herein as the “normal” human dosing regimen for Talzenna™, regardless of the disorder treated. Any dosing regimen having a shorter duration (e.g., 21 days) or involving the administration of less Talzenna™ (e.g., 0.5 mg/day) is referred to herein as a “reduced” human dosing regimen. Examples of reduced human dosing regimens include the following: (i) 0.9 mg/day; (ii) 0.8 mg/day; (iii) 0.7 mg/day; (iv) 0.6 mg/day; (v) 0.5 mg/day; (vi) 0.4 mg/day; (vii) 0.3 mg/day; (viii) 0.2 mg/day; or (ix) 0.1 mg/day.

### Example 5: Dosing Regimens for CCR8 Targeting Agent and PARPi

A human patient may be treated according to the following regimen. One of olaparib, niraparib, rucaparib or talazoparib (PARPi) is orally administered according to one of the dosing regimens listed in Example 2, accompanied by intravenous administration of a radioconjugated CCR8 targeting agent as detailed herein in either single or fractional administration. For example, the dosing regimens include, by way of example: (a) the PARPi and the radioconjugated CCR8 targeting agent administered concurrently, wherein (i) each is administered beginning on the same day, (ii) the radioconjugated CCR8 targeting agent is administered in a single dose or fractionated doses not less than one week apart, and (iii) the PARPi is administered daily or twice daily (as appropriate), and for a duration equal to or exceeding that of the radioconjugated CCR8 targeting agent administration; or (b) the PARPi and radioconjugated CCR8 targeting agent are administered concurrently, wherein (i) the PARPi administration precedes radioconjugated CCR8 targeting agent administration by at least one week, (ii) the radioconjugated CCR8 targeting agent is administered in a single dose or fractionated doses not less than one week apart, and (iii) the PARPi is administered daily or twice daily (as appropriate), and for a duration equal to or exceeding that of the radioconjugated CCR8 targeting agent administration.

### Example 6: Dosing Regimens for CCR8 Targeting Agent and a CD47 Blockade

The CD47 blocking agent may be a monoclonal antibody or fusion protein that prevents CD47 binding to SIRPα. Such CD47 blockades include at least magrolimab, lemzoparlimab, AO-176, TTI-621, TTI-622, ALX148 or any combination thereof. The CD47 blockade may alternatively, or additionally, include agents that modulate the expression of CD47 and/or SIRPα, such as phosphorodiamidate morpholino oligomers (PMO) that block translation of CD47, for example, MBT-001, or small molecules that downregulate CD47/SIRPα activity such as RRx-001. Therapeutically effective doses of anti-CD47 antibodies include at least 0.05-10 mg/kg. Thus, methods of the present disclosure may include administering one or more of the anti-CD47 antibodies or other agents, accompanied by intravenous administration of a radioconjugated CCR8 targeting agent as detailed herein in either single or fractional dose administration. For example, the dosing regimens include, by way of example: (a) the anti-CD47 antibody or agent and the radioconjugated CCR8 targeting agent administered concurrently, wherein (i) each is administered beginning on the same day, (ii) the radioconjugated CCR8 targeting agent is administered in a single dose or fractionated doses not less than one week apart, and (iii) the anti-CD47 antibody or agent is administered daily or twice daily (as appropriate), and for a duration equal to or exceeding that of the radioconjugated CCR8 targeting agent administration; or (b) the anti-CD47 antibody or agent and radioconjugated CCR8 targeting agent are administered concurrently, wherein (i) the anti-CD47 antibody or agent administration precedes radioconjugated CCR8 targeting agent administration by at least one week, (ii) the radioconjugated CCR8 targeting agent is administered in a single dose or fractionated doses not less than one week apart, and (iii) the anti-CD47 antibody or agent is administered daily or twice daily (as appropriate), and for a duration equal to or exceeding that of the radioconjugated CCR8 targeting agent administration.

### Example 7: Dosing Regimens for CCR8 Targeting Agent and an ICI

The immune checkpoint inhibitor (ICI) may, for example, be a monoclonal antibody against any of PD-1, PD-L1, PD-L2, CTLA-4, TIM3, LAG3 or VISTA. Therapeutically effective doses of these antibodies include at least 0.05-10 mg/kg. Thus, methods of the present disclosure include administering one or more ICI, accompanied by intravenous administration of a radioconjugated CCR8 targeting agent as detailed herein in either single or fractional administration. For example, the dosing regimens include, by way of example: (a) the ICI and the radioconjugated CCR8 targeting agent administered concurrently, wherein (i) each is administered beginning on the same day, (ii) the radioconjugated CCR8 targeting agent is administered in a single dose or fractionated doses not less than one week apart, and (iii) the ICI is administered daily or twice daily (as appropriate), and for a duration equal to or exceeding that of the radioconjugated CCR8 targeting agent administration; or (b) the ICI and radioconjugated CCR8 targeting agent are administered concurrently, wherein (i) the anti-CD47 antibody administration precedes radioconjugated CCR8 targeting agent administration by at least one week, (ii) the radioconjugated CCR8 targeting agent is administered in a single dose or fractionated doses not less than one week apart, and (iii) the ICI is administered daily or twice daily (as appropriate), and for a duration equal to or exceeding that of the radioconjugated CCR8 targeting agent administration.

### Example 8: Methods for Testing Therapeutic Combination Approaches

Combining Radioconjugated Anti-CCR8 mAb with an ICI (PD-1 Inhibitor):

BALB/c mice will be injected subcutaneously in the right flank with 2×10⁶ CT26 cells. When tumors grow to approximately 100 mm³, mice will be treated intravenously with various doses of radioconjugated anti-CCR8 antibody (0 nCi, MED, and MTD ²²⁵Ac as defined by the dose escalation study; 500 ng total amount of IgG; clone SA214G2), in the presence or absence of an antibody that blocks the function of PD-1 (e.g., clone RMP1-14). Weights and tumor volumes will be measured twice a week, and a Kaplan-Meier survival curve will be generated. In a separate experiment, tumor infiltration and activation of immune cells (e.g., monocytes, macrophages, T cells, NK cells, neutrophils) can be quantified using flow cytometry to determine if addition of PD-1 blockade activates an antitumor immune response to a greater extent compared to anti-CCR8 treatment alone.

Combining Radioconjugated Anti-CCR8 mAb with CD47 Blockade:

BALB/c mice will be injected subcutaneously in the right flank with 2×10⁶ CT26 cells. When tumors grow to approximately 100 mm³, mice will be treated intravenously with various doses of radioconjugated anti-CCR8 antibody (0 nCi, MED, and MTD ²²⁵Ac; 500 ng total amount of IgG; clone SA214G2), in the presence or absence of an antibody that blocks the function of CD47 (e.g., clone MIAP301). Weights and tumor volumes will be measured twice a week, and a Kaplan-Meier survival curve will be generated. In a separate experiment, tumor infiltration and activation of immune cells (e.g., monocytes, macrophages, T cells, NK cells, neutrophils) can be quantified using flow cytometry to determine if addition of CD47 blockade activates an antitumor immune response to a greater extent compared to treatment with the radioconjugated CCR8 targeting agent alone.

Combining Radioconjugated Anti-CCR8 mAb with an Antibody Targeting MICA:

BALB/c mice will be injected subcutaneously in the right flank with 2×10⁶ CT26-MICA cells. (These cells express human MICA on the cell surface.) When tumors grow to approximately 100 mm³, mice will be treated intravenously with various doses of radioconjugated anti-CCR8 antibody (0 nCi, MED, and MTD ²²⁵Ac; 500 ng total amount of IgG; clone SA214G2), in the presence or absence of an antibody that binds to and stabilizes MICA on the cell surface (e.g., clone 7C6). Weights and tumor volumes will be measured twice a week, and a Kaplan-Meier survival curve will be generated.

Combining Radioconjugated Anti-CCR8 mAb with CAR T Cell Therapy:

BALB/c mice will be injected in the tail vein with 5×10⁵ CT26.GUCY2C cells. (These cells express GUCY2C, a cancer antigen expressed on intestinal malignancies; Magee, 2016.) After receiving non-myeloablative total body irradiation, mice will then be administered murine CAR T cells directed against GUCY2C (Magee, 2016), in the presence or absence of radioconjugated anti-CCR8 antibody (0 nCi, MED, and MTD ²²⁵Ac; 500 ng total amount of IgG; clone SA214G2). Weights and tumor volumes will be measured twice a week, and a Kaplan-Meier survival curve will be generated.

Without limitation, the following aspects of the invention are also provided by this disclosure:

Aspect 1. A method for treating a solid cancer in a subject, the method including: administering to the subject a therapeutically effective amount of a radiolabeled CCR8 targeting agent.

Aspect 2. The method according to any preceding aspect, wherein the solid cancer is a breast cancer, gastric cancer, bladder cancer, cervical cancer, endometrial cancer, skin cancer, stomach cancer, testicular cancer, esophageal cancer, bronchioloalveolar cancer, prostate cancer, colorectal cancer, ovarian cancer, cervical epidermoid cancer, pancreatic cancer, lung cancer, renal cancer, head and neck cancer, or any combination thereof.

Aspect 3. The method according to any preceding aspect, wherein the solid cancer is colorectal cancer, gastric cancer, ovarian cancer, non-small cell lung carcinoma, head and neck squamous cell cancer, pancreatic cancer, renal cancer, or any combination thereof.

Aspect 4. The method according to any preceding aspect, wherein the solid cancer is a tumor including tumor infiltrating CCR8-positive Tregs.

Aspect 5. The method according to any preceding aspect, wherein the radiolabeled CCR8 targeting agent includes a radiolabel selected from ¹³¹I, ¹²⁵I, ¹²⁴I, ¹²³I, ⁹⁰Y, ¹⁷⁷Lu, ¹⁶Re, ¹⁸⁸Re, ⁸⁹Sr, ¹⁵³Sm, ³²P, ²²⁵Ac, ²¹³Bi, ²¹³Po, ²¹¹At, ²¹²Bi, ²¹³Bi, ²²³Ra, ²²⁷Th, ¹⁴⁹Tb, ¹³⁷Cs, ²¹²Pb or ¹⁰³Pd, or any combination thereof.

Aspect 6. The method according to any preceding aspect, wherein the radiolabeled CCR8 targeting agent includes a radiolabel selected from ¹³¹I, ⁹⁰Y, ¹⁷⁷Lu, ²²⁵Ac, ²¹³Bi, ²¹¹At, ²¹³Bi, ²²⁷Th, ²¹²Pb, or any combination thereof.

Aspect 7. The method according to any preceding aspect, wherein the radiolabeled CCR8 targeting agent includes an antibody against CCR8, such as against human CCR8.

Aspect 8. The method according to any preceding aspect, wherein the radiolabeled CCR8 targeting agent is a monoclonal antibody or an antigen-binding fragment of a monoclonal antibody,

Aspect 9. The method according to any preceding aspect, wherein the radiolabeled CCR8 targeting agent includes a monoclonal antibody selected from FPA175 of Five Prime Therapeutics, HBM1022 of Harbour Biomed, SFR114 of Surface Oncology, BMS-986340 of Bristol-Meyers Squib, JTX-1811 of Jounce Therapeutics, SA214G2 of BioLegend, CBL712 of Creative Biolabs, or chimeric or humanized versions thereof, or CCR8-binding antibodies including the heavy chain CDRs and/or the light chain CDRs of any of said antibodies, or CCR8-binding antibodies including the heavy chain variable region and/or the light chain variable region of any of said antibodies, or CCR8-binding antibody fragments of any of the aforementioned antibodies, or any combination thereof

Aspect 10. The method according to any preceding aspect, wherein the effective amount of the radiolabeled CCR8 targeting agent is a maximum tolerated dose.

Aspect 11. The method according to any preceding aspect, wherein the radiolabeled CCR8 targeting agent is ²²⁵Ac-, ¹⁷⁷Lu-, or ¹³¹I-labeled.

Aspect 12. The method according to any preceding aspect, wherein the therapeutically effective amount of the radiolabeled CCR8 targeting agent includes a single dose that delivers less than 2Gy, or less than 8 Gy, such as doses of 2 Gy to 8 Gy, to the subject.

Aspect 13. The method according to any preceding aspect, wherein the radiolabeled CCR8 targeting agent is ²²⁵Ac-labeled, and the effective amount of the ²²⁵Ac-labeled CCR8 targeting agent includes a dose of 0.1 to 50 μCi/kg body weight of the subject, or 0.2 to 20 μCi/kg body weight of the subject, or 0.5 to 10 μCi/kg subject body weight.

Aspect 14. The method according to any preceding aspect, wherein the radiolabeled CCR8 targeting agent is a full-length antibody against CCR8 that is ²²⁵Ac-labeled, and the effective of the ²²⁵Ac-labeled CCR8 targeting agent includes less than 5 μCi/kg body weight of the subject, such as 0.1 to 5 μCi/kg body weight of the subject.

Aspect 15. The method according to any preceding aspect, wherein the radiolabeled CCR8 targeting agent is an antibody fragment, such as a Fab, Fab₂, scFv, a minibody, a sdAb, or a nanobody against CCR8 that is ²²⁵Ac-labeled, and the effective of the ²²⁵Ac-labeled CCR8 targeting agent includes greater than 5 μCi/kg body weight of the subject, such as 5 to 20 μCi/kg body weight of the subject.

Aspect 16. The method according to any one of aspects 1 to 12, wherein the radiolabeled CCR8 targeting agent is ²²⁵Ac-labeled, and the effective amount of the ²²⁵Ac-labeled CCR8 targeting agent includes 2 μCi to 2mCi, or 2 μCi to 250 μCi, or 75 μCi to 400 μCi.

Aspect 17. The method according to any one of aspects 1 to 12, wherein the radiolabeled CCR8 targeting agent is ¹⁷⁷Lu-labeled and the effective amount of the CCR8 targeting agent includes a dose of less than 1000 μCi/kg body weight of the subject, such as a dose of 1 to 900 μCi/kg body weight of the subject, or 5 to 250 μCi/kg body weight of the subject or 50 to 450 μCi/kg body weight.

Aspect 18. The method according to any one of aspects 1 to 12, wherein the radiolabeled CCR8 targeting agent is ¹⁷⁷Lu-labeled, and the effective amount of the ¹⁷⁷Lu-labeled CCR8 targeting agent includes a dose of 10 mCi to at or below 30 mCi, or from at least 100 μCi to at or below 3 mCi, or from 3 mCi to at or below 30 mCi.

Aspect 19. The method according to any one of aspects 1 to 12, wherein the radiolabeled CCR8 targeting agent is ¹³¹I-labeled, and the effective amount of the ¹³¹I-labeled CCR8 targeting agent includes a dose of less than 1200 mCi, such as a dose of 25 to 1200 mCi, or 100 to 400 mCi, or 300 to 600 mCi, or 500 to 1000 mCi.

Aspect 20. The method according to any one of aspects 1 to 12, wherein the radiolabeled CCR8 targeting agent is ¹³¹I-labeled, and the effective amount of the ¹³¹I-labeled CCR8 targeting agent includes a dose of less than 200 mCi, such as a dose of 1 to 200 mCi, or 25 to 175 mCi, or 50 to 150 mCi.

Aspect 21. The method according to any preceding aspect, wherein the effective amount of the radiolabeled CCR8 targeting agent includes a protein dose of less than 3 mg/kg body weight of the subject, such as from 0.001 mg/kg patient weight to 3.0 mg/kg patient weight, or from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight.

Aspect 22. The method according to any preceding aspect, wherein the radiolabeled CCR8 targeting agent is administered according to a dosing schedule selected from the group consisting of once every 7, 10, 12, 14, 20, 24, 28, 36, and 42 days throughout a treatment period, wherein the treatment period includes at least two doses.

Aspect 23. The method according to any one of aspects 1 to 6, wherein the radiolabeled CCR8 targeting agent is a peptide or small molecule.

Aspect 24. The method according to any preceding aspect, further including administering to the subject a therapeutically effective amount of an immune checkpoint therapy, a chemotherapeutic agent, a DNA damage response inhibitor (DDRi), a CD47 blockade, a MICA blockade, an adoptive cell therapy, or any combination thereof.

Aspect 25. The method according to aspect 24, wherein the immune checkpoint therapy includes an antibody (such as a full-length antibody or an antigen-binding antibody fragment) against CTLA-4, PD-1, TIM-3, VISTA, BTLA, LAG-3, TIGIT, CD28, OX40, GITR, CD137, CD40, CD40L, CD27, HVEM, PD-L1, PD-L2, PD-L3, PD-L4, CD80, CD86, CD137-L, GITR-L, CD226, B7-H3, B7-H4, BTLA, TIGIT, GALS, KIR, 2B4, CD160, CGEN-15049, or any combination thereof.

Aspect 26. The method according to aspect 24, wherein the immune checkpoint therapy includes an antibody against PD-1, PD-L1, PD-L2, CTLA-4, CD137, or any combination thereof.

Aspect 27. The method according to aspect 24, wherein the DDRi includes a poly(ADP-ribose) polymerase inhibitor (PARPi), an ataxia telangiectasia mutated inhibitor (ATMi), an ataxia talangiectasia mutated and Rad-3 related inhibitor (ATRi), or a Wee1 inhibitor.

Aspect 28. The method according to aspect 27, wherein the PARPi includes one or more of olaparib, niraparib, rucaparib and talazoparib.

Aspect 29. The method according to aspect 27, wherein the ATMi includes one or more of KU-55933, KU-59403, wortmannin, CP466722, or KU-60019.

Aspect 30. The method according to aspect 27, wherein the ATRi includes one or more of Schisandrin B, NU6027, NVP-BEA235, VE-821, VE-822, AZ20, or AZD6738.

Aspect 31. The method according to aspect 27, wherein the Wee1 inhibitor includes AZD-1775 (i.e., adavosertib).

Aspect 32. The method according to aspect 24, wherein the CD47 blockade includes a monoclonal antibody or an antigen-binding fragment thereof that prevents CD47 binding to SIRPα and/or an agent that modulates CD47 expression and/or a small molecule that diminishes CD47 checkpoint activity.

Aspect 33. The method according to aspect 32, wherein the CD47 blockade includes magrolimab, lemzoparlimab, AO-176, TTI-621, TTI-622, ALX148, RRx-001, an agent modulates CD47 expression includes phosphorodiamidate morpholino oligomers (PMO) that block translation of CD47 (e.g., MBT-001), or any combination thereof.

Aspect 34. The method according to aspect 32, wherein the therapeutically effective amount of the CD47 blockade includes 0.05 to 5 mg/Kg patient weight.

Aspect 35. The method according to aspect 24, wherein the MICA blockade includes clone IPH43 or HYB3-24302 from Creative Biolabs, or anti-MICA antibodies against the alpha-3 domain.

Aspect 36. The method according to aspect 24, wherein the adoptive cell therapy includes administering to the subject an effective amount of a population of cells expressing a chimeric antigen receptor or a T-cell receptor (CAR/TCR), such as a genetically engineered or recombinant TCR.

Aspect 37. The method according to aspect 36, wherein the population of cells expressing the CAR/TCR are autologous cells or allogeneic cells.

Aspect 38. The method according to 36 or 37, wherein the population of cells expressing the CAR/TCR target (via recognition by the CAR or TCR), CD19, CD20, CD22, CD30, CD33, CD38, CD123, CD138, CS-1, B-cell maturation antigen (BCMA), MAGEA3, MAGEA3/A6, KRAS, CLL1, MUC-1, HER2, EpCam, GD2, GPA7, PSCA, EGFR, EGFRvIII, ROR1, mesothelin, CD33/TL3Ra, c-Met, CD37, PSMA, Glycolipid F77, GD-2, gp100, NY-ESO-1 TCR, FRalpha, CD24, CD44, CD133, CD166, CA-125, HE4, Oval, estrogen receptor, progesterone receptor, uPA, PAI-1, MICA, MICB, ULBP1, ULBP2, ULBP3, ULBP4, ULBP5 or ULBP6, or any combination thereof.

Aspect 39. The method according to any one of aspects 36 to 38, wherein the population of cells expressing the CAR/TCR includes gene-edited CAR T-cells, and wherein the gene-edited CAR T-cells fail to properly express at least one checkpoint receptor, at least one T-cell receptor, or both of the at least one checkpoint receptor and the at least one T-cell receptor.

Aspect 40. The method according to aspect 24, wherein the radiolabeled CCR8 targeting agent is administered before the immune checkpoint therapy and/or the DDRi and/or the CD47 blockade and/or MICA blockade and/or adoptive cell therapy; or wherein the immune checkpoint therapy and/or the DDRi and/or CD47 blockade and/or MICA blockade and/or adoptive cell therapy is administered before the CCR8 targeting agent.

Aspect 41. The method according to aspect 24, wherein the radiolabeled CCR8 targeting agent is administered before and/or after administration of the immune checkpoint therapy and/or the DDRi and/or the CD47 blockade and/or MICA blockade and/or adoptive cell therapy.

Aspect 42. The method according to aspect 24, wherein the radiolabeled CCR8 targeting agent is administered simultaneously with, or temporally overlapping with, administration of the immune checkpoint therapy and/or the DDRi and/or the CD47 blockade and/or MICA blockade and/or adoptive cell therapy.

Aspect 43. The method according to any preceding aspect, wherein the radiolabeled CCR8 targeting agent is a multi-specific antibody, wherein the multi-specific antibody includes: a first target recognition component which specifically binds to an epitope of CCR8, and a second target recognition component which specifically binds to a different epitope of CCR8 than the first target recognition component, or an epitope of a different antigen.

Aspect 44. A method for treating a proliferative disease or disorder, the method including: diagnosing the subject with CCR8-positive Treg cells; and if the subject has CCR8-positive Treg cells, administering to the subject a therapeutically effective amount of a radiolabeled CCR8 targeting agent according to any of the methods of aspects 1 to 38.

Aspect 45. The method according to aspect 44, wherein the diagnosing includes obtaining a sample of tissue from the subject; mounting the sample on a substrate; and detecting the presence or absence of CCR8 antigen using a diagnostic antibody, wherein the diagnostic antibody includes an antibody against CCR8 labeled with a radiolabel such as ³H, ¹⁴C, ³²P, ³⁵S, and ¹²⁵⁷I; fluorescent or chemiluminescent compounds, such as fluorescein isothiocyanate, rhodamine, or luciferin; or an enzyme, such as alkaline phosphatase, β-galactosidase, or horseradish peroxidase.

Aspect 46. The method according to aspect 44, wherein the diagnosing includes administering a radiolabeled CCR8 targeting agent to the subject, wherein the CCR8 targeting agent includes a radiolabel such as including ¹⁸F, ¹¹C, ⁶⁸Ga, ⁶⁴Cu, ⁸⁹Zr, ¹²⁴, ^{99m}Tc, or ¹¹¹In; and, after a time sufficient to allow the administered radiolabeled CCR8 targeting agent to distribute and bind CCR8 that may expressed at tissue sites (such as within tumors), imaging the tissues with a non-invasive imaging technique to detect the presence, absence and/or localization of CCR8-positive cells.

Aspect 47. The method according to aspect 46, wherein the non-invasive imaging technique includes positron emission tomography (PET imaging) for ¹⁸F, ¹¹C, ⁶⁸Ga, ⁶⁴Cu, ⁸⁹Zr, or ¹²⁴I labeled CCR8 targeting agents or single photon emission computed tomography (SPECT imaging) for ^{99m}Tc or ¹¹¹In labeled CCR8 targeting agents.

Any and all publications, patents, patent applications and other documents cited in this application are hereby incorporated by reference in their entireties for all purposes to the same extent as if each individual publication, patent, patent application or other document were individually indicated to be incorporated by reference for all purposes.

It should be understood that wherever in this disclosure an aspect or embodiment of the invention or an element or step thereof is described in terms of “including,” “include(s),” “comprising,” or “comprise(s),” corresponding aspects, embodiments, elements or steps thereof expressed, instead, in terms of “consisting essentially of” or “consisting of” are also intended to be disclosed and provided by this disclosure.

While various specific embodiments have been illustrated and described herein, it will be appreciated that various changes can be made without departing from the spirit and scope of the invention(s). Moreover, features described in connection with one embodiment of the invention may be used in conjunction with other embodiments, even if not explicitly exemplified in combination within.

### REFERENCES

Bagley S J, Desai A S, Linette G P, June C H, O'Rourke D M. CAR T cell therapy for glioblastoma: recent clinical advances and future challenges. Neuro Oncol. 2018; 20(11):1429-1438.
Barsheshet Y, Wildbaum G, Levy E, et al. CCR8+FOXp3+ Treg cells as master drivers of immune regulation. Proc Natl Acad Sci. 2017; 114(23):6086-6091.
Bell M M, Gutsche N T, King A P, et al. Glypican-3-Targeted Alpha Particle Therapy for Hepatocellular Carcinoma. Molecules. 2020; 26(1):4.
Cao X, Cai S F, Fehniger T A, et al. Granzyme B and Perforin Are Important for Regulatory T Cell-Mediated Suppression of Tumor Clearance. Immunity. 2007; 27(4):635-646.
Couper K N, Lanthier P A, Perona-Wright G, et al. Anti-CD25 Antibody-Mediated Depletion of Effector T Cell Populations Enhances Susceptibility of Mice to Acute but Not Chronic Toxoplasma gondii Infection. J Immunol. 2009; 182(7):3985-3994.
Dar T B, Henson R M, Shiao S L. Targeting Innate Immunity to Enhance the Efficacy of Radiation Therapy. Front Immunol. 2019; 9(JAN):1-11.
Dawicki W, et al. Daratumumab-225 Actinium conjugate demonstrates greatly enhanced antitumor activity against experimental multiple myeloma tumors. Oncoimmunology. 2019; 8(8):e1607673.
Dépis F, Hu C, Weaver J, et al. Abstract 4532: Preclinical evaluation of JTX-1811, an anti-CCR8 antibody with enhanced ADCC activity, for preferential depletion of tumor-infiltrating regulatory T cells. In: Cancer Research. Vol 80. American Association for Cancer Research (AACR); 2020:4532-4532.
De Simone M, Arrigoni A, Rossetti G, et al. Transcriptional Landscape of Human Tissue Lymphocytes Unveils Uniqueness of Tumor-Infiltrating T Regulatory Cells. Immunity. 2016; 45(5):1135-1147.
Drago J Z, Modi S, Chandarlapaty S. Unlocking the potential of antibody-drug conjugates for cancer therapy. Nat Rev Clin Oncol. Published online Feb. 8, 2021.
Epperly R, Gottschalk S, Velasquez M P. A Bump in the Road: How the Hostile AML Microenvironment Affects CAR T Cell Therapy. Front Oncol. 2020; 10:262.
Eyquem, et al. Targeting a CAR to the TRAC locus with CRISPR/Cas9 enhances tumour rejection. Nature. 2017, 543:113-117.
Fridman W H, Pagès F, Sau\grave{t}s-Fridman C, Galon J. The immune contexture in human tumours: Impact on clinical outcome. Nat Rev Cancer. 2012; 12(4):298-306.
Garg R, Allen K J H, Dawicki W, Geoghegan E M, Ludwig D L, Dadachova E. ²²⁵Ac-labeled CD33-targeting antibody reverses resistance to Bcl-2 inhibitor venetoclax in acute myeloid leukemia models. Cancer Medicine. 2021; 10:1128-1140.
Han G, et al. Tim-3: an activation marker and activation limiter of innate immune cells. Front Immunol. 2013; 4:449.
Haberkorn U, Giesel F, Morgenstern A, Kratochwil C. The future of radioligand therapy: α, β, or Both? J Nucl Med. 2017; 58(7):1017-1018.
Hellmann M D, Ciuleanu T-E, Pluzanski A, et al. Nivolumab plus Ipilimumab in Lung Cancer with a High Tumor Mutational Burden. N Engl J Med. 2018; 378(22):2093-2104.
Hoelzinger D B, Smith S E, Mirza N, Dominguez A L, Manrique S Z, Lustgarten J. Blockade of CCL1 Inhibits T Regulatory Cell Suppressive Function Enhancing Tumor Immunity without Affecting T Effector Responses. J Immunol. 2010; 184(12):6833-6842.
Kato, et al. Regulation of the expression of MHC class I-related chain A, B (MICA, MICB) via chromatin remodeling and its impact on the susceptibility of leukemic cells to the cytotoxicity of NKG2D-expressing cells. Leukemia. 2007; 21:2103-08.
Kawashima A, Kanazawa T, Yoshida T, et al. MP18-02 identification of ccr8 as a specific marker of tumor tissue-infiltrating regulatory t cells and its possibility as a therapeutic target in renal cell carcinoma. J Urol. 2020; 203:e234.
Lake A, Warren M, Das S, et al. 726 SRF 114 is a fully human, CCR8 selective IgG1 antibody that induces destruction of tumor Tregs through ADCC. J Immunother Cancer. 2020; 8(Suppl 3):A769-A769.
Lamichhane P, Amin N, Agarwal M, Lamichhane N. Checkpoint Inhibition: Will Combination with Radiotherapy and Nanoparticle-Mediated Delivery Improve Efficacy?Medicines. 2018; 5, 114.
Lämmermann T, Kastenmüller W. Concepts of GPCR-controlled navigation in the immune system. Immunol Rev. 2019; 289(1):205-231.
Lan R, Jhatakia A, Campbell J, et al. Abstract 6694: Highly selective anti-CCR8 antibody-mediated depletion of regulatory T cells leads to potent antitumor activity alone and in combination with anti-PD-1 in preclinical models. In: Cancer Research. Vol 80. American Association for Cancer Research (AACR); 2020:6694-6694.
Liu H, Wang S, Xin J, Wang J, Yao C, Zhang Z. Role of NKG2D and its ligands in cancer immunotherapy. Am J Cancer Res. 2019; 9(10):2064-2078.
Lu S, Hu S, Gan X, et al. 711 HBM1022, a novel anti-CCR8 antibody depletes tumor-infiltrating regulatory T cells via enhanced ADCC activity, mediates potent anti-tumor activity with Keytruda. J Immunother Cancer. 2020; 8(Suppl 3):A753-A753.
Magee M S, Kraft C L, Abraham T S, et al. GUCY2C-directed CAR-T cells oppose colorectal cancer metastases without autoimmunity. Oncoimmunology. 2016; 5(10):e1227897.
Magnuson A M, Kiner E, Ergun A, et al. Identification and validation of a tumor-infiltrating Treg transcriptional signature conserved across species and tumor types. Proc Natl Acad Sci USA. 2018; 115(45):E10672-E10681.
Maleki Vareki S, Garrigós C, Duran I. Biomarkers of response to PD-1/PD-L1 inhibition. Crit Rev Oncol Hematol. 2017; 116:116-124.
Marabelle A, Kohrt H, Sagiv-Barfi I, et al. Depleting tumor-specific Tregs at a single site eradicates disseminated tumors. J Clin Invest. 2013; 123(6):2447-2463.
Mardiana S, Solomon B J, Darcy P K, Beavis P A. Supercharging adoptive T cell therapy to overcome solid tumor-induced immunosuppression. Sci Transl Med. 2019; 11(495):2293.
Melaiu O, Lucarini V, Cifaldi L, Fruci D. Influence of the Tumor Microenvironment on NK Cell Function in Solid Tumors. Front Immunol. 2020; 10:3038.
Nelson B, Andersson J, Wuest F. Targeted Alpha Therapy: Progress in Radionuclide Production, Radiochemistry, and Applications. Pharmaceutics. 2020; 13(1):49.
Neti PVS V, Howell R W. Log normal distribution of cellular uptake of radioactivity: implications for biologic responses to radiopharmaceuticals. J Nucl Med. 2006; 47(6):1049-1058.
Nishikawa H, Sakaguchi S. Regulatory T cells in tumor immunity. Int J Cancer. 2010; 127(4):759-767.
Ogbomo H, Cinatl J, Mody C H, Forsyth P A. Immunotherapy in gliomas: Limitations and potential of natural killer (NK) cell therapy. Trends Mol Med. 2011; 17(8):433-441.
Ohue Y, Nishikawa H. Regulatory T (Treg) cells in cancer: Can Treg cells be a new therapeutic target? Cancer Sci. 2019; 110(7):2080-2089.
Onizuka S, Tawara I, Shimizu J, Sakaguchi S, Fujita T, Nakayama E. Tumor rejection by in vivo administration of anti-CD25 (interleukin-2 receptor a) monoclonal antibody. Cancer Res. 1999; 59(13):3128-3133.
Papazahariadou, et al. Involvement of NK cells against tumors and parasites. Int. J. Biol. Markers. 2007; 22:144-53.
Paschen, et al. Differential Clinical Significance of Individual NKG2D Ligands in Melanoma: Soluble ULBP2 as an Indicator of Poor Prognosis Superior to S100B. Clin. Cancer Res. 2009; 15:5208-15.
Peggs K S, Quezada S A, Chambers C A, Korman A J, Allison J P. Blockade of CTLA-4 on both effector and regulatory T cell compartments contributes to the antitumor activity of anti-CTLA-4 antibodies. J Exp Med. 2009; 206(8):1717-1725.
Plitas G, Konopacki C, Wu K, et al. Regulatory T Cells Exhibit Distinct Features in Human Breast Cancer. Immunity. 2016; 45(5):1122-1134.
Rankin A, Naik E. 861 Development of FPA157, an anti-CCR8 depleting antibody engineered to preferentially eliminate tumor-infiltrating T regulatory cells. J Immunother Cancer. 2020; 8(Suppl 3):A914-A914.
Ren, et. al. Multiplex Genome Editing to Generate Universal CART Cells Resistant to PD1 Inhibition. Clin. Cancer Res. 2017, 23:2255-2266.
Saleh R, Elkord E. FoxP3+ T regulatory cells in cancer: Prognostic biomarkers and therapeutic targets. Cancer Lett. 2020; 490:174-185.
Santoni, et al. Natural Killer (NK) Cells from Killers to Regulators: Distinct Features Between Peripheral Blood and Decidual NK Cells. Am. J. Reprod Immunol. 2007; 58:280-88.
Sharabi A B, Lim M, DeWeese T L, Drake C G. Radiation and checkpoint blockade immunotherapy: radiosensitisation and potential mechanisms of synergy. Lancet Oncol. 2015; 16(13):e498-509.
Shimizu J, Yamazaki S, Sakaguchi S. Induction of tumor immunity by removing CD25+CD4+ T cells: a common basis between tumor immunity and autoimmunity, J. Immunol 1999; 163(10):5211-8.
Simpson T R, Li F, Montalvo-Ortiz W, et al. Fc-dependent depletion of tumor-infiltrating regulatory t cells co-defines the efficacy of anti-CTLA-4 therapy against melanoma. J Exp Med. 2013; 210(9):1695-1710.
Smyth M J, Teng M W L, Swann J, Kyparissoudis K, Godfrey D I, Hayakawa Y. CD4+CD25+ T Regulatory Cells Suppress NK Cell-Mediated Immunotherapy of Cancer. J Immunol. 2006; 176(3):1582-1587.
Takimoto C H, Chao M P, Gibbs C, et al. The Macrophage “Do not eat me” signal, CD47, is a clinically validated cancer immunotherapy target. Ann Oncol. 2019; 30(3):486-489.
Tanaka A, Sakaguchi S. Targeting Treg cells in cancer immunotherapy. Eur J Immunol. 2019; 49(8):eji.201847659.
Twyman-Saint Victor C et al. Radiation and dual checkpoint blockade activate non-redundant immune mechanisms in cancer. Nature. 2015; 520:373-377.
Van Damme H, Dombrecht B, Kiss M, et al. Therapeutic depletion of CCR8+ tumor-infiltrating regulatory T cells elicits antitumor immunity and synergizes with anti-PD-1 therapy. J Immunother Cancer. 2021; 9(2):e001749.
Vignali D A A, Collison L W, Workman C J. How regulatory T cells work. Nat Rev Immunol. 2008; 8(7):523-532.
Villarreal D O, L'Huillier A, Armington S, et al. Targeting CCR8 induces protective antitumor immunity and enhances vaccine-induced responses in colon cancer. Cancer Res. 2018; 78(18):5340-5348.
Wang Z, Chen W, Zhang X, Cai Z, Huang W. A long way to the battlefront: CAR T cell therapy against solid cancers. J Cancer. 2019; 10(14):3112-3123.
Wang Y, Liu Z-G, Yuan H, et al. The Reciprocity between Radiotherapy and Cancer Immunotherapy. Clin Cancer Res. 2019; 25(6):1709-1717.
Xing S, Ferrari de Andrade L. NKG2D and MICA/B shedding: a ‘tag game’ between NK cells and malignant cells. Clin Transl Immunol. 2020; 9(12).
Zheng C, Zheng L, Yoo J K, et al. Landscape of Infiltrating T Cells in Liver Cancer Revealed by Single-Cell Sequencing. Cell. 2017; 169(7):1342-1356.e16.

1. A method for treating a solid cancer in a subject, the method comprising:
administering to the subject a therapeutically effective amount of a radiolabeled CCR8 targeting agent, wherein the radiolabeled CCR8 targeting agent comprises a radiolabel selected from ²²⁵Ac, ¹⁷⁷Lu, ¹³¹I, ⁹⁰Y, ²¹³Bi, ²¹¹At, ²¹³Bi, ²²⁷Th, ²¹²Pb, or any combination thereof.

2. The method of claim 1, wherein the solid cancer is a breast cancer, gastric cancer, bladder cancer, cervical cancer, endometrial cancer, skin cancer, stomach cancer, testicular cancer, esophageal cancer, bronchioloalveolar cancer, prostate cancer, colorectal cancer, ovarian cancer, cervical epidermoid cancer, pancreatic cancer, lung cancer, renal cancer, head and neck cancer, or any combination thereof.

3. The method of claim 1, wherein the therapeutically effective amount of the radiolabeled CCR8 targeting agent is an amount effective to deplete or ablate CCR8-positive Treg cells in the solid cancer, wherein the depletion or ablation is not mediated by ADCC.

4. The method of claim 3, wherein the therapeutically effective amount of the radiolabeled CCR8 targeting agent is an amount at least 10-fold lower than an unconjugated CCR8 targeting agent, or an amount at least 20-fold lower than the unconjugated CCR8 targeting agent, or an amount at least 30-fold lower than the unconjugated CCR8 targeting agent.

5. The method of claim 1, wherein the therapeutically effective amount of the radiolabeled CCR8 targeting agent is an amount effective to increase the amount and/or activity of immune cells that produce antitumor immunity.

6. The method of claim 1, wherein the radioisotope radiolabeled CCR8 targeting agent is ²²⁵Ac-labeled, and the therapeutically effective amount of the ²²⁵Ac-labeled CCR8 targeting agent comprises:
a protein dose of less than 3 mg/kg body weight of the subject, such as from 0.001 mg/kg patient weight to 3.0 mg/kg patient weight, or from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight; and
a radiation dose of 0.1 to 50 μCi/kg body weight of the subject, or 0.1 to 5 μCi/kg body weight of the subject, or 5 to 20 μCi/kg subject body weight.

7. The method of claim 1, wherein the radioisotope radiolabeled CCR8 targeting agent is ²²⁵Ac-labeled, and the therapeutically effective amount of the ²²′Ac-labeled CCR8 targeting agent comprises:
a protein dose of less than 3 mg/kg body weight of the subject, such as from 0.001 mg/kg patient weight to 3.0 mg/kg patient weight, or from 0.005 mg/kg patient weight to 2.0 mg/kg patient weight, or from 0.01 mg/kg patient weight to 1 mg/kg patient weight, or from 0.1 mg/kg patient weight to 0.6 mg/kg patient weight, or 0.3 mg/kg patient weight, or 0.4 mg/kg patient weight, or 0.5 mg/kg patient weight, or 0.6 mg/kg patient weight; and
a radiation dose of 2 μCi to 2mCi, or 2 μCi to 250 μCi, or 75 μCi to 400 μCi.

8. The method of claim 1, wherein the therapeutically effective amount of the radiolabeled CCR8 targeting agent is administered as a single dose.

9. The method of claim 1, wherein the radiolabeled CCR8 targeting agent is administered according to a dosing schedule selected from the group consisting of once every 7, 10, 12, 14, 20, 24, 28, 36, and 42 days throughout a treatment period, wherein the treatment period includes at least two doses.

10. The method of claim 1, further comprising:
administering to the subject a therapeutically effective amount of an immune checkpoint therapy, a MICA blockade, a CD47 blockade, or any combination thereof.

11. The method of claim 10, wherein the immune checkpoint therapy comprises an antibody against PD-1, PD-L1, PD-L2, CTLA-4, TIM3, LAG3, VISTA, or any combination thereof.

12. The method of claim 10, wherein the CD47 blockade comprises magrolimab, lemzoparlimab, AO-176, TTI-621, TTI-622, ALX148, RRx-001, an agent that modulates CD47 expression, MBT-001, or any combination thereof.

13. The method of claim 10, wherein the MICA blockade comprises a monoclonal antibody against MICA that stabilizes surface expression of MICA on tumor cells in the subject.

14. The method of claim 1, further comprising:
administering to the subject a therapeutically effective amount of a DNA damage response inhibitor (DDRi), a chemotherapeutic agent, or any combination thereof.

15. The method of claim 14, wherein the DDRi comprises a poly(ADP-ribose) polymerase inhibitor (PARPi), an ataxia telangiectasia mutated inhibitor (ATMi), an ataxia talangiectasia mutated and Rad-3 related inhibitor (ATRi), or a Wee1 inhibitor.

16. The method of claim 14, wherein the chemotherapeutic agent is a radiosensitizer.

17. The method of claim 1, further comprising:
performing adoptive cell therapy on the subject to treat the solid cancer.

18. The method of claim 17, wherein the adoptive cell therapy comprises administering gene-edited CAR-T cells to the subject, wherein the gene-edited CAR-T cells fail to properly express at least one checkpoint receptor and/or at least one T cell receptor.

19. The method of claim 17, wherein the adoptive cell therapy comprises administering to the subject an effective amount of a population of cells expressing a chimeric antigen receptor or an engineered T-cell receptor (CAR/TCR).

20. The method of claim 19, wherein the population of cells expressing the CAR/TCR target CD19, CD20, CD22, CD30, CD33, CD38, CD123, CD138, CS-1, B-cell maturation antigen (BCMA), MAGEA3, MAGEA3/A6, KRAS, CLL1, MUC-1, HER2, EpCam, GD2, GPA7, PSCA, EGFR, EGFRvIII, ROR1, mesothelin, CD33/IL3Ra, c-Met, CD37, PSMA, Glycolipid F77, GD-2, gp100, NY-ESO-1 TCR, FRalpha, CD24, CD44, CD133, CD166, CA-125, HE4, Oval, estrogen receptor, progesterone receptor, uPA, PAI-1, MICA, MICB, ULBP1, ULBP2, ULBP3, ULBP4, ULBP5 or ULBP6, or any combination thereof.

21. The method of claim 17, wherein the adoptive cell therapy is performed simultaneous to administration of the radiolabeled CCR8 targeting agent, or is performed 4, 5, 6, 7, or 8 days after administration of the radiolabeled CCR8 targeting agent.

22. The method of claim 1, further comprising:
administering a radiolabeled CD33 targeting agent to the subject.

23. The method of claim 22, wherein the radiolabeled CD33 targeting agent is a radiolabeled anti-CD33 antibody or a radiolabeled CD33-binding antibody fragment.

24-39. (canceled)


SEQUENCE LISTING (319 sequences)

SEQ ID NO: 1 - protein, 121 aa, Artificial Sequence
  Synthetic sequence
VQLVESGGGLVQPGRSLRLSCAASGFTFDDYAMHWVRQAPGKGLEWVSGISWNSGSIGYADSVKGRFTISRDNSKNSLYLQMNSLRAEDTALYYCARGRESYRVSLRFDYWGQGTLVTVSS

SEQ ID NO: 2 - skipped

SEQ ID NO: 3 - protein, 108 aa, Artificial Sequence
  Synthetic sequence
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSAWVFGGGTKLT

SEQ ID NO: 4 - skipped

SEQ ID NO: 5 - protein, 5 aa, Artificial Sequence
  Synthetic sequence
DYAMH

SEQ ID NO: 6 - protein, 17 aa, Artificial Sequence
  Synthetic sequence
GISWNSGSIGYADSVKG

SEQ ID NO: 7 - protein, 13 aa, Artificial Sequence
  Synthetic sequence
GRESYRVSLRFDY

SEQ ID NO: 8 - protein, 13 aa, Artificial Sequence
  Synthetic sequence
SGSSSNIGNNYVS

SEQ ID NO: 9 - protein, 7 aa, Artificial Sequence
  Synthetic sequence
DNNKRPS

SEQ ID NO: 10 - protein, 11 aa, Artificial Sequence
  Synthetic sequence
GTWDSSLSAWV

SEQ ID NO: 11 - protein, 118 aa, Artificial Sequence
  Anti-CCR8-2 Heavy Chain
EVQLLESGGGLVQPGGSLRLSCAAGGFTFSAYTMNWVRQAPGKGLEWVSAISASGGRTYYADSVKGRFTISRDNSKNTLYLQMNSLRAEDTAVYYCARRFARGWFDPWGQGTLVTVSS

SEQ ID NO: 12 - skipped

SEQ ID NO: 13 - protein, 108 aa, Artificial Sequence; identical to SEQ ID NO: 3
  Anti-CCR8-2 Light Chain

SEQ ID NO: 14 - skipped

SEQ ID NO: 15 - protein, 5 aa, Artificial Sequence
  Synthetic sequence
AYTMN

SEQ ID NO: 16 - protein, 17 aa, Artificial Sequence
  Synthetic sequence
AISASGGRTYYADSVKG

SEQ ID NO: 17 - protein, 9 aa, Artificial Sequence
  Synthetic sequence
RFARGWFDP

SEQ ID NO: 18 - protein, 13 aa, Artificial Sequence
  Synthetic sequence
SGSSSNIGNNYVS

SEQ ID NO: 19 - protein, 7 aa, Artificial Sequence
  Synthetic sequence
DNNKRPS

SEQ ID NO: 20 - protein, 11 aa, Artificial Sequence
  Synthetic sequence
GTWDSSLSAWV

SEQ ID NO: 21 - protein, 122 aa, Artificial Sequence
  Anti-CCR8-2-4 Heavy Chain, Anti-CCR8-2-5 Heavy Chain
EVQLVESGGGLVQPGRSLRLSCAASGFTFDDYAMHWVRQAPGKGLEWVSGISWNSGSIGYADSVKGRFTISRDNSKNSLYLQMNSLRAEDTALYYCARGRKSYRVSLRFDYWGQGTLVTVSS

SEQ ID NO: 22 - skipped

SEQ ID NO: 23 - protein, 108 aa, Artificial Sequence; identical to SEQ ID NO: 3
  Anti-CCR8-2-4 Light Chain, Anti-CCR8-2-5 Light Chain

SEQ ID NO: 24 - skipped

SEQ ID NO: 25 - protein, 5 aa, Artificial Sequence
  Synthetic sequence
DYAMH

SEQ ID NO: 26 - protein, 17 aa, Artificial Sequence
  Synthetic sequence
GISWNSGSIGYADSVKG

SEQ ID NO: 27 - protein, 13 aa, Artificial Sequence
  Synthetic sequence
GRKSYRVSLRFDY

SEQ ID NO: 28 - protein, 13 aa, Artificial Sequence
  Synthetic sequence
SGSSSNIGNNYVS

SEQ ID NO: 29 - protein, 7 aa, Artificial Sequence
  Synthetic sequence
DNNKRPS

SEQ ID NO: 30 - protein, 11 aa, Artificial Sequence
  Synthetic sequence
GTWDSSLSAWV

SEQ ID NO: 31 - protein, 122 aa, Artificial Sequence
  Anti-CCR8-2-7 Heavy Chain
EVQLVESGGGLVQPGRSLRLSCAASGFTFDDYAMHWVRQAPGKGLEWVSGISWNSGSIGYADSVKGRFTISRDNSKNSLYLQMNSLRAEDTALYYCARGRDSYRKSLRFDYWGQGTLVTVSS

SEQ ID NO: 32 - skipped

SEQ ID NO: 33 - protein, 110 aa, Artificial Sequence
  Anti-CCR8-2-7 Light Chain
QSVLTQPPSVSGAPGQRVTISCTGSGSNIGNNYVSWYQQLPGTAPKMLIYDNTRRPSGIPDRFSGSKSDTSATLGITGLQTGDEADYYCGAWDSSLRMWVFGGGTKLTVL

SEQ ID NO: 34 - skipped

SEQ ID NO: 35 - protein, 5 aa, Artificial Sequence
  Synthetic sequence
DYAMH

SEQ ID NO: 36 - protein, 17 aa, Artificial Sequence
  Synthetic sequence
GISWNSGSIGYADSVKG

SEQ ID NO: 37 - protein, 13 aa, Artificial Sequence
  Synthetic sequence
GRDSYRKSLRFDY

SEQ ID NO: 38 - protein, 13 aa, Artificial Sequence
  Synthetic sequence
TGSGSNIGNNYVS

SEQ ID NO: 39 - protein, 7 aa, Artificial Sequence
  Synthetic sequence
DNTRRPS

SEQ ID NO: 40 - protein, 11 aa, Artificial Sequence
  Synthetic sequence
GAWDSSLRMWV

SEQ ID NO: 41 - protein, 119 aa, Artificial Sequence
  Anti-CCR8-1 Heavy Chain
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARAVRNRFRFDYWGQGTLVTVSS

SEQ ID NO: 42 - skipped

SEQ ID NO: 43 - protein, 111 aa, Artificial Sequence
  Anti-CCR8-1 Light Chain
QSALTQPASVSGSPGQSITISCTGTSSDVGSYNLVSWYQQHPGKAPKLMIYEVSKRPSGVSNRFSGSKSGNTASLTISGLQAEDEADYYCSSYAGSSTFVVFGGGTKLTVL

SEQ ID NO: 44 - skipped

SEQ ID NO: 45 - protein, 5 aa, Artificial Sequence
  Synthetic sequence
SYYMH

SEQ ID NO: 46 - protein, 17 aa, Artificial Sequence
  Synthetic sequence
IINPSGGSTSYAQKFQG

SEQ ID NO: 47 - protein, 10 aa, Artificial Sequence
  Synthetic sequence
AVRNRFRFDY

SEQ ID NO: 48 - protein, 14 aa, Artificial Sequence
  Synthetic sequence
TGTSSDVGSYNLVS

SEQ ID NO: 49 - protein, 7 aa, Artificial Sequence
  Synthetic sequence
EVSKRPS

SEQ ID NO: 50 - protein, 11 aa, Artificial Sequence
  Synthetic sequence
SSYAGSSTFVV

SEQ ID NO: 51 - protein, 118 aa, Artificial Sequence
  Anti-CCR8-2-1 Heavy Chain
EVQLLESGGGLVQPGGSLRLSCAARGFIFSGYTMLWVRQAPGKGLEWVSAITASGGRTYYADSVKGRFTISRDNSKNTLYLQMNSLRAEDTAVYYCARRFARGWFDPWGQGTLVTVSS

SEQ ID NO: 52 - skipped

SEQ ID NO: 53 - protein, 108 aa, Artificial Sequence; identical to SEQ ID NO: 3
  Anti-CCR8-2-1 Light Chain

SEQ ID NO: 54 - skipped

SEQ ID NO: 55 - protein, 5 aa, Artificial Sequence
  Synthetic sequence
GYTML

SEQ ID NO: 56 - protein, 17 aa, Artificial Sequence
  Synthetic sequence
AITASGGRTYYADSVKG

SEQ ID NO: 57 - protein, 9 aa, Artificial Sequence
  Synthetic sequence
RFARGWFDP

SEQ ID NO: 58 - protein, 13 aa, Artificial Sequence
  Synthetic sequence
SGSSSNIGNNYVS

SEQ ID NO: 59 - protein, 7 aa, Artificial Sequence
  Synthetic sequence
DNNKRPS

SEQ ID NO: 60 - protein, 11 aa, Artificial Sequence
  Synthetic sequence
GTWDSSLSAWV

SEQ ID NO: 61 - protein, 122 aa, Artificial Sequence
  Anti-CCR8-2-9 Heavy Chain
EVQLVESGGGLVQPGRSLRLSCAASGFTFDDYAMHWVRQAPGKGLEWVSGISWNSGSIGYADSVKGRFTISRDNSKNSLYLQMNSLRAEDTALYYCARGRKSYRDSLRFDYWGQGTLVTVSS

SEQ ID NO: 62 - skipped

SEQ ID NO: 63 - protein, 110 aa, Artificial Sequence; identical to SEQ ID NO: 33
  Anti-CCR8-2-9 Light Chain

SEQ ID NO: 64 - skipped

SEQ ID NO: 65 - protein, 5 aa, Artificial Sequence
  Synthetic sequence
DYAMH

SEQ ID NO: 66 - protein, 17 aa, Artificial Sequence
  Synthetic sequence
GISWNSGSIGYADSVKG

SEQ ID NO: 67 - protein, 13 aa, Artificial Sequence
  Synthetic sequence
GRKSYRDSLRFDY

SEQ ID NO: 68 - protein, 13 aa, Artificial Sequence
  Synthetic sequence
TGSGSNIGNNYVS

SEQ ID NO: 69 - protein, 7 aa, Artificial Sequence
  Synthetic sequence
DNTRRPS

SEQ ID NO: 70 - protein, 11 aa, Artificial Sequence
  Synthetic sequence
GAWDSSLRMWV

SEQ ID NO: 71 - protein, 122 aa, Artificial Sequence
  Anti-CCR8-2-8 Heavy Chain
EVQLVESGGGLVQPGRSLRLSCAASGFTFDDYAMHWVRQAPGKGLEWVSGISWNSGSIGYADSVKGRFTISRDNSKNSLYLQMNSLRAEDTALYYCARGRRSYRDSLRFDYWGQGTLVTVSS

SEQ ID NO: 72 - skipped

SEQ ID NO: 73 - protein, 110 aa, Artificial Sequence; identical to SEQ ID NO: 33
  Anti-CCR8-2-8 Light Chain

SEQ ID NO: 74 - skipped

SEQ ID NO: 75 - protein, 5 aa, Artificial Sequence
  Synthetic sequence
DYAMH

SEQ ID NO: 76 - protein, 17 aa, Artificial Sequence
  Synthetic sequence
GISWNSGSIGYADSVKG

SEQ ID NO: 77 - protein, 13 aa, Artificial Sequence
  Synthetic sequence
GRRSYRDSLRFDY

SEQ ID NO: 78 - protein, 13 aa, Artificial Sequence
  Synthetic sequence
TGSGSNIGNNYVS

SEQ ID NO: 79 - protein, 7 aa, Artificial Sequence
  Synthetic sequence
DNTRRPS

SEQ ID NO: 80 - protein, 11 aa, Artificial Sequence
  Synthetic sequence
GAWDSSLRMWV

SEQ ID NO: 81 - protein, 122 aa, Artificial Sequence; identical to SEQ ID NO: 61
  Anti-CCR8-2-10 Heavy Chain

SEQ ID NO: 82 - skipped

SEQ ID NO: 83 - protein, 110 aa, Artificial Sequence
  Anti-CCR8-2-10 Light Chain
QSVLTQPPSVSAAPGQKVTISCSGTSSNIGKNFVSWYQQLPGTAPKLLIYDDNKRPSGIPDRFSGSKSATSATLGITGLQTGDGADYYCGTWDSSLSAWVFGGGTKLTVL

SEQ ID NO: 84 - skipped

SEQ ID NO: 85 - protein, 5 aa, Artificial Sequence
  Synthetic sequence
DYAMH

SEQ ID NO: 86 - protein, 17 aa, Artificial Sequence
  Synthetic sequence
GISWNSGSIGYADSVKG

SEQ ID NO: 87 - protein, 13 aa, Artificial Sequence
  Synthetic sequence
GRKSYRDSLRFDY

SEQ ID NO: 88 - protein, 13 aa, Artificial Sequence
  Synthetic sequence
SGTSSNIGKNFVS

SEQ ID NO: 89 - protein, 7 aa, Artificial Sequence
  Synthetic sequence
DDNKRPS

SEQ ID NO: 90 - protein, 11 aa, Artificial Sequence
  Synthetic sequence
GTWDSSLSAWV

SEQ ID NO: 91 - protein, 122 aa, Artificial Sequence; identical to SEQ ID NO: 61
  Anti-CCR8-2-3 Heavy Chain

SEQ ID NO: 92 - skipped

SEQ ID NO: 93 - protein, 108 aa, Artificial Sequence; identical to SEQ ID NO: 3
  Anti-CCR8-2-3 Light Chain

SEQ ID NO: 94 - skipped

SEQ ID NO: 95 - protein, 5 aa, Artificial Sequence
  Synthetic sequence
DYAMH

SEQ ID NO: 96 - protein, 17 aa, Artificial Sequence
  Synthetic sequence
GISWNSGSIGYADSVKG

SEQ ID NO: 97 - protein, 13 aa, Artificial Sequence
  Synthetic sequence
GRKSYRDSLRFDY

SEQ ID NO: 98 - protein, 13 aa, Artificial Sequence
  Synthetic sequence
SGSSSNIGNNYVS

SEQ ID NO: 99 - protein, 7 aa, Artificial Sequence
  Synthetic sequence
DNNKRPS

SEQ ID NO: 100 - protein, 11 aa, Artificial Sequence
  Synthetic sequence
GTWDSSLSAWV

SEQ ID NO: 101 - protein, 116 aa, Artificial Sequence
  Anti-CCR8-1-1 Heavy Chain
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGVGNGFRFDYWGQGTLVT

SEQ ID NO: 102 - skipped

SEQ ID NO: 103 - protein, 111 aa, Artificial Sequence
  Anti-CCR8-1-1 Light Chain
QSALTQPPSVSGSPGQSITISCTGTSSDVGTYNLVSWYQQHPGNAPKLMIYEVTKRPSGVSNRFSGSKSGNTATLTISGLQAEDEADYHCSSYAGSITHVVFGGGTKLTVL

SEQ ID NO: 104 - skipped

SEQ ID NO: 105 - protein, 5 aa, Artificial Sequence
  Synthetic sequence
SYYMH

SEQ ID NO: 106 - protein, 17 aa, Artificial Sequence
  Synthetic sequence
IINPSGGSTSYAQKFQG

SEQ ID NO: 107 - protein, 10 aa, Artificial Sequence
  Synthetic sequence
GVGNGFRFDY

SEQ ID NO: 108 - protein, 14 aa, Artificial Sequence
  Synthetic sequence
TGTSSDVGTYNLVS

SEQ ID NO: 109 - protein, 7 aa, Artificial Sequence
  Synthetic sequence
EVTKRPS

SEQ ID NO: 110 - protein, 11 aa, Artificial Sequence
  Synthetic sequence
SSYAGSITHVV

SEQ ID NO: 111 - protein, 116 aa, Artificial Sequence; identical to SEQ ID NO: 101
  Anti-CCR8-1-2 Heavy Chain

SEQ ID NO: 112 - skipped

SEQ ID NO: 113 - protein, 112 aa, Artificial Sequence
  Anti-CCR8-1-2 Light Chain
QSALTQPASVSGSPGQSITISCTGTSGDVGSYSLVSWYQHHPSRAPKLIIYEVNKRPSGVSDRFSGSKSGNTASLTITGLQAEDEAHYFCSSYTGNINLPVVFGGGTKLTVL

SEQ ID NO: 114 - skipped

SEQ ID NO: 115 - protein, 5 aa, Artificial Sequence
  Synthetic sequence
SYYMH

SEQ ID NO: 116 - protein, 17 aa, Artificial Sequence
  Synthetic sequence
IINPSGGSTSYAQKFQG

SEQ ID NO: 117 - protein, 10 aa, Artificial Sequence
  Synthetic sequence
GVGNGFRFDY

SEQ ID NO: 118 - protein, 14 aa, Artificial Sequence
  Synthetic sequence
TGTSGDVGSYSLVS

SEQ ID NO: 119 - protein, 7 aa, Artificial Sequence
  Synthetic sequence
EVNKRPS

SEQ ID NO: 120 - protein, 12 aa, Artificial Sequence
  Synthetic sequence
SSYTGNINLPVV

SEQ ID NO: 121 - protein, 116 aa, Artificial Sequence; identical to SEQ ID NO: 101
  Anti-CCR8-1-3 Heavy Chain

SEQ ID NO: 122 - skipped

SEQ ID NO: 123 - protein, 111 aa, Artificial Sequence
  Anti-CCR8-1-3 Light Chain
QSALTQPPSVSGSPGQSITISCSGTSSDVGIYNLVSWYQQHPGKAPKLIIYEVIKRPSGISNRFSGFKSGNTASLTISGLQAEDEADYYCSSYAGPVTYVVFGGGTKLTVL

SEQ ID NO: 124 - skipped

SEQ ID NO: 125 - protein, 5 aa, Artificial Sequence
  Synthetic sequence
SYYMH

SEQ ID NO: 126 - protein, 17 aa, Artificial Sequence
  Synthetic sequence
IINPSGGSTSYAQKFQG

SEQ ID NO: 127 - protein, 10 aa, Artificial Sequence
  Synthetic sequence
GVGNGFRFDY

SEQ ID NO: 128 - protein, 14 aa, Artificial Sequence
  Synthetic sequence
SGTSSDVGIYNLVS

SEQ ID NO: 129 - protein, 7 aa, Artificial Sequence
  Synthetic sequence
EVIKRPS

SEQ ID NO: 130 - protein, 11 aa, Artificial Sequence
  Synthetic sequence
SSYAGPVTYVV

SEQ ID NO: 131 - protein, 116 aa, Artificial Sequence; identical to SEQ ID NO: 101
  Anti-CCR8-1-4 Heavy Chain

SEQ ID NO: 132 - skipped

SEQ ID NO: 133 - protein, 111 aa, Artificial Sequence
  Anti-CCR8-1-4 Light Chain
QSALTQPASVSGSPGQSITISCSGTSSNIGKYNLVSWYQQHPGEAPTLLIYEATKRPSGVSNRFSGSKSGNTASLTISGLQAEDEADYICSSYAGSRVFVVFGGGTKLTVL

SEQ ID NO: 134 - skipped

SEQ ID NO: 135 - protein, 5 aa, Artificial Sequence
  Synthetic sequence
SYYMH

SEQ ID NO: 136 - protein, 17 aa, Artificial Sequence
  Synthetic sequence
IINPSGGSTSYAQKFQG

SEQ ID NO: 137 - protein, 10 aa, Artificial Sequence
  Synthetic sequence
GVGNGFRFDY

SEQ ID NO: 138 - protein, 14 aa, Artificial Sequence
  Synthetic sequence
SGTSSNIGKYNLVS

SEQ ID NO: 139 - protein, 7 aa, Artificial Sequence
  Synthetic sequence
EATKRPS

SEQ ID NO: 140 - protein, 11 aa, Artificial Sequence
  Synthetic sequence
SSYAGSRVFVV

SEQ ID NO: 141 - protein, 116 aa, Artificial Sequence; identical to SEQ ID NO: 101
  Anti-CCR8-1-5 Heavy Chain

SEQ ID NO: 142 - protein, 6 aa, Artificial Sequence
  Synthetic sequence
HHHHHH

SEQ ID NO: 143 - protein, 111 aa, Artificial Sequence
  Anti-CCR8-1-5 Light Chain
QSALTQPPSVSGSPGQSITISCSGTSSDVGSYNLVSWYQQEPGKAPKLIIYEVNKRPSGVSNRFSGSKSGNTASLTISGLQAEDEADYHCSSYAGSSTYVVFGGGTKLTVL

SEQ ID NO: 144 - skipped

SEQ ID NO: 145 - protein, 5 aa, Artificial Sequence
  Synthetic sequence
SYYMH

SEQ ID NO: 146 - protein, 17 aa, Artificial Sequence
  Synthetic sequence
IINPSGGSTSYAQKFQG

SEQ ID NO: 147 - protein, 10 aa, Artificial Sequence
  Synthetic sequence
GVGNGFRFDY

SEQ ID NO: 148 - protein, 14 aa, Artificial Sequence
  Synthetic sequence
SGTSSDVGSYNLVS

SEQ ID NO: 149 - protein, 7 aa, Artificial Sequence
  Synthetic sequence
EVNKRPS

SEQ ID NO: 150 - protein, 11 aa, Artificial Sequence
  Synthetic sequence
SSYAGSSTYVV

SEQ ID NO: 151 - protein, 119 aa, Artificial Sequence
  Anti-CCR8-2-6 Heavy Chain
EVQLVESGGGLVQPGRSLRLSCAASGFTFDDYAMHWVRQAPGKGLEWVSGISWNSGSIGYADSVKGRFTISRDNSKNSLYLQMNSLRAEDTALYYCARGRVSYRESLRFDYWGQGTLVT

SEQ ID NO: 152 - skipped

SEQ ID NO: 153 - protein, 108 aa, Artificial Sequence; identical to SEQ ID NO: 3
  Anti-CCR8-2-6 Light Chain

SEQ ID NO: 154 - skipped

SEQ ID NO: 155 - protein, 5 aa, Artificial Sequence
  Synthetic sequence
DYAMH

SEQ ID NO: 156 - protein, 17 aa, Artificial Sequence
  Synthetic sequence
GISWNSGSIGYADSVKG

SEQ ID NO: 157 - protein, 13 aa, Artificial Sequence
  Synthetic sequence
GRVSYRESLRFDY

SEQ ID NO: 158 - protein, 13 aa, Artificial Sequence
  Synthetic sequence
SGSSSNIGNNYVS

SEQ ID NO: 159 - protein, 7 aa, Artificial Sequence
  Synthetic sequence
DNNKRPS

SEQ ID NO: 160 - protein, 11 aa, Artificial Sequence
  Synthetic sequence
GTWDSSLSAWV

SEQ ID NO: 161 - protein, 115 aa, Artificial Sequence
  Anti-CCR8-2-2 Heavy Chain
EVQLLESGGGLVQPGGSLRLSCAAGGFTFSAYTMNWVRQAPGKGLEWVSAISASGGRTYYADSVKGRFTISRDNSKNTLYLQMNSLRAEDTAVYYCARRFARGWFDPWGQGTLVT

SEQ ID NO: 162 - skipped

SEQ ID NO: 163 - protein, 110 aa, Artificial Sequence
  Anti-CCR8-2-2 Light Chain
QSVLTQPPSVSAAPGQRVTISCSGSTSNIGNHYVSWYQQLPRAVPKLVIYDNDKRPSGISDRFSGSRSGTSATLDISGLQAGDEADYYCATWDYSLTAVVFGGGTKLTVL

SEQ ID NO: 164 - skipped

SEQ ID NO: 165 - protein, 5 aa, Artificial Sequence
  Synthetic sequence
AYTMN

SEQ ID NO: 166 - protein, 17 aa, Artificial Sequence
  Synthetic sequence
AISASGGRTYYADSVKG

SEQ ID NO: 167 - protein, 9 aa, Artificial Sequence
  Synthetic sequence
RFARGWFDP

SEQ ID NO: 168 - protein, 13 aa, Artificial Sequence
  Synthetic sequence
SGSTSNIGNHYVS

SEQ ID NO: 169 - protein, 7 aa, Artificial Sequence
  Synthetic sequence
DNDKRPS

SEQ ID NO: 170 - protein, 11 aa, Artificial Sequence
  Synthetic sequence
ATWDYSLTAVV

SEQ ID NO: 171 - protein, 355 aa, Homo sapiens
  Human CCR8 Sequence
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 172 - protein, 35 aa, Homo sapiens
  N-terminal extracellular domain of human CCR8
MDYTLDLSVTTVTDYYYPDIFSSPCDAELIQTNGK

SEQ ID NO: 173 - protein, 16 aa, Artificial Sequence
  light chain CDR1
RSSKSLLHSNGNTYLY

SEQ ID NO: 174 - protein, 7 aa, Artificial Sequence
  light chain CDR2
RMSNLAS

SEQ ID NO: 175 - protein, 9 aa, Artificial Sequence
  light chain CDR3
MQHLEYPLT

SEQ ID NO: 176 - protein, 5 aa, Artificial Sequence
  heavy chain CDR1
TYALY

SEQ ID NO: 177 - protein, 19 aa, Artificial Sequence
  heavy chain CDR2
RIRSKSNNYATYYADSVKD

SEQ ID NO: 178 - protein, 14 aa, Artificial Sequence
  heavy chain CDR3
ARFYYSDYGYAMDY

SEQ ID NO: 179 - protein, 5 aa, Mus musculus
SHWLH

SEQ ID NO: 180 - protein, 17 aa, Mus musculus
VLDPSDFYSNYNQNFKG

SEQ ID NO: 181 - protein, 11 aa, mus musculus
GLLSGDYAMDY

SEQ ID NO: 182 - protein, 16 aa, mus musculus
RSSQSIVHSNGNTYLE

SEQ ID NO: 183 - protein, 7 aa, mus musculus
KVSNRFS

SEQ ID NO: 184 - protein, 9 aa, mus musculus
FQGSYVPWT

SEQ ID NO: 185 - protein, 120 aa, mus musculus
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGAGTSVTVSS

SEQ ID NO: 186 - protein, 112 aa, mus musculus
DVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIK

SEQ ID NO: 187 - protein, 463 aa, mus musculus
MGWSCIIVLLVSTATGVHSQVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 188 - protein, 238 aa, mus musculus
MKLPVRLLVLMFWIPASSSDVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 189 - protein, 444 aa, mus musculus
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 190 - protein, 219 aa, mus musculus
DVLMTQIPLSLPVSLGDQASISCRSSQSIVHSNGNTYLEWYLQKPGQSPKSLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 191 - protein, 10 aa, mus musculus
QVQLQQPGAE

SEQ ID NO: 192 - protein, 10 aa, mus musculus
DVLMTQIPLS

SEQ ID NO: 193 - protein, 5 aa, Artificial Sequence
  Description of Artificial Sequence: Synthetic peptide
DYAMS

SEQ ID NO: 194 - protein, 17 aa, Artificial Sequence
  Description of Artificial Sequence: Synthetic peptide
TISDGGTYTYYPDSVKG

SEQ ID NO: 195 - protein, 10 aa, Artificial Sequence
  Description of Artificial Sequence: Synthetic peptide
EWGDYDGFDY

SEQ ID NO: 196 - protein, 11 aa, Artificial Sequence
  Description of Artificial Sequence: Synthetic peptide
RASQEISGYLS

SEQ ID NO: 197 - protein, 7 aa, Artificial Sequence
  Description of Artificial Sequence: Synthetic peptide
AASTLDS

SEQ ID NO: 198 - protein, 9 aa, Artificial Sequence
  Description of Artificial Sequence: Synthetic peptide
LQYDSYPYT

SEQ ID NO: 199 - protein, 119 aa, Artificial Sequence
  Description of Artificial Sequence: Synthetic polypeptide
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSS

SEQ ID NO: 200 - protein, 107 aa, Artificial Sequence
  Description of Artificial Sequence: Synthetic polypeptide
DIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIK

SEQ ID NO: 201 - protein, 471 aa, Artificial Sequence
  Description of Artificial Sequence: Synthetic polypeptide
MDMRVPAQLLGLLLLWLRGARCQVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 236 aa, Artificial Sequence
  Description of Artificial Sequence: Synthetic polypeptide
MDMRVPAQLLGLLLLWLRGARCDIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 5 aa, Artificial Sequence
  Description of Artificial Sequence: Synthetic peptide
SHWLH

SEQ ID NO: 204 - protein, 17 aa, Artificial Sequence
  Description of Artificial Sequence: Synthetic peptide
VLDPSDFYSNYNQNFKG

SEQ ID NO: 205 - protein, 11 aa, Artificial Sequence
  Description of Artificial Sequence: Synthetic peptide
GLLSGDYAMDY

SEQ ID NO: 206 - protein, 16 aa, Artificial Sequence
  Description of Artificial Sequence: Synthetic peptide
RSSQSIVHSNGNTYLE

SEQ ID NO: 207 - protein, 7 aa, Artificial Sequence
  Description of Artificial Sequence: Synthetic peptide
KVSNRFS

SEQ ID NO: 208 - protein, 9 aa, Artificial Sequence
  Description of Artificial Sequence: Synthetic peptide
FQGSYVPWT

SEQ ID NO: 209 - protein, 120 aa, Artificial Sequence
  Description of Artificial Sequence: Synthetic polypeptide
QVQLQQPGAELVRPGTSVKLSCKASGYTFTSHWLHWVKQRPGQGLEWIGVLDPSDFYSNYNQNFKGKATLTVDTSSSTAYMQLSSLTSEDSAVYYCARGLLSGDYAMDYWGQGTSVTVSS

SEQ ID NO: 210 - protein, 112 aa, Artificial Sequence; identical to SEQ ID NO: 186
  Description of Artificial Sequence: Synthetic polypeptide

SEQ ID NO: 211 - protein, 463 aa, Artificial Sequence; identical to SEQ ID NO: 187
  Description of Artificial Sequence: Synthetic polypeptide

SEQ ID NO: 212 - protein, 238 aa, Artificial Sequence; identical to SEQ ID NO: 188
  Description of Artificial Sequence: Synthetic polypeptide

SEQ ID NO: 213 - protein, 7 aa, Artificial Sequence
  Description of Artificial Sequence: Synthetic peptide
TFGLSVG

SEQ ID NO: 214 - protein, 16 aa, Artificial Sequence
  Description of Artificial Sequence: Synthetic peptide
HIWWDDDKYYNPALKS

SEQ ID NO: 215 - protein, 10 aa, Artificial Sequence
  Description of Artificial Sequence: Synthetic peptide
IGADALPFDY

SEQ ID NO: 216 - protein, 16 aa, Artificial Sequence
  Description of Artificial Sequence: Synthetic peptide
RSSKSLLHSNGNTYLY

SEQ ID NO: 217 - protein, 7 aa, Artificial Sequence
  Description of Artificial Sequence: Synthetic peptide
RMSNLAS

SEQ ID NO: 218 - protein, 9 aa, Artificial Sequence
  Description of Artificial Sequence: Synthetic peptide
MQHLEYPFT

SEQ ID NO: 219 - protein, 120 aa, Artificial Sequence
  Description of Artificial Sequence: Synthetic polypeptide
QVTLKESGPGILRPSQTLSLTCSFSGFSLSTFGLSVGWIRQPSGKGLEWLAHIWWDDDKYYNPALKSRLTISKDTSKNQVFLKIANVDTADTATYYCARIGADALPFDYWGQGTTLTVSS

SEQ ID NO: 220 - protein, 112 aa, Artificial Sequence
  Description of Artificial Sequence: Synthetic polypeptide
DIVLTQTAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 221 - protein, 475 aa, Artificial Sequence
  Description of Artificial Sequence: Synthetic polypeptide
MGRLTSSFLLLIVPAYVLSQVTLKESGPGILRPSQTLSLTCSFSGFSLSTFGLSVGWIRQPSGKGLEWLAHIWWDDDKYYNPALKSRLTISKDTSKNQVFLKIANVDTADTATYYCARIGADALPFDYWGQGTTLTVSSAKTTPPSVYPLAPGCGDTTGSSVTSGCLVKGYFPEPVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYTLPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 222 - protein, 239 aa, Artificial Sequence
  Description of Artificial Sequence: Synthetic polypeptide
MRCLAEFLGLLVLWIPGAIGDIVLTQTAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 223 - protein, 5 aa, Artificial Sequence
  Description of Artificial Sequence: Synthetic peptide
DHIIH

SEQ ID NO: 224 - protein, 17 aa, Artificial Sequence
  Description of Artificial Sequence: Synthetic peptide
YIYPRDGYIKYNEKFKG

SEQ ID NO: 225 - protein, 8 aa, Artificial Sequence
  Description of Artificial Sequence: Synthetic peptide
GYYYAMDY

SEQ ID NO: 226 - protein, 16 aa, Artificial Sequence
  Description of Artificial Sequence: Synthetic peptide
RSSQSIVHSIGNTYLE

SEQ ID NO: 227 - protein, 9 aa, Artificial Sequence
  Description of Artificial Sequence: Synthetic peptide
FQGSHVPFT

SEQ ID NO: 228 - protein, 117 aa, Artificial Sequence
  Description of Artificial Sequence: Synthetic polypeptide
QVQLQQSDAELVKPGASVKISCKVSGYTFTDHIIHWMKQRPEQGLEWIGYIYPRDGYIKYNEKFKGKATLTADKSSSTAYMQVNSLTSEDSAVYFCARGYYYAMDYWGQGTSVTVSS

SEQ ID NO: 229 - protein, 112 aa, Artificial Sequence
  Description of Artificial Sequence: Synthetic polypeptide
DVLMTQTPLSLPVSLGDQASISCRSSQSIVHSIGNTYLEWYLQKPGQSPKLLIYKVSNRFSGVPERFSGSGSGTDFTLKISRVEAEDLGVYYCFQGSHVPFTFGSGTKLEIK

SEQ ID NO: 230 - protein, 460 aa, Artificial Sequence
  Description of Artificial Sequence: Synthetic polypeptide
MEWSWVSLFFLSVTTGVHSQVQLQQSDAELVKPGASVKISCKVSGYTFTDHIIHWMKQRPEQGLEWIGYIYPRDGYIKYNEKFKGKATLTADKSSSTAYMQVNSLTSEDSAVYFCARGYYYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 231 - protein, 238 aa, Artificial Sequence
  Description of Artificial Sequence: Synthetic polypeptide
MKLPVRLLVLMFWIPASRSDVLMTQTPLSLPVSLGDQASISCRSSQSIVHSIGNTYLEWYLQKPGQSPKLLIYKVSNRFSGVPERFSGSGSGTDFTLKISRVEAEDLGVYYCFQGSHVPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 232 - protein, 5 aa, Artificial Sequence
  Description of Artificial Sequence: Synthetic peptide
SYWMH

SEQ ID NO: 233 - protein, 17 aa, Artificial Sequence
  Description of Artificial Sequence: Synthetic peptide
MIDPSDVYTNYNPKFKG

SEQ ID NO: 234 - protein, 6 aa, Artificial Sequence
  Description of Artificial Sequence: Synthetic peptide
NYSGDY

SEQ ID NO: 235 - protein, 115 aa, Artificial Sequence
  Description of Artificial Sequence: Synthetic polypeptide
QVQLLQPGAELVRPGTSVKLSCKTSGYTFSSYWMHWVKQRPGQGLEWIGMIDPSDVYTNYNPKFKGKATLTVDTSSSTAYMQLSSLTSEDSAVYYCARNYSGDYWGQGTTLTVSS

SEQ ID NO: 236 - protein, 112 aa, Artificial Sequence
  Description of Artificial Sequence: Synthetic polypeptide
DVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIK

SEQ ID NO: 237 - protein, 458 aa, Artificial Sequence
  Description of Artificial Sequence: Synthetic polypeptide
MGWSCIIVLLVSTATCVHSQVQLLQPGAELVRPGTSVKLSCKTSGYTFSSYWMHWVKQRPGQGLEWIGMIDPSDVYTNYNPKFKGKATLTVDTSSSTAYMQLSSLTSEDSAVYYCARNYSGDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 238 - protein, 238 aa, Artificial Sequence
  Description of Artificial Sequence: Synthetic polypeptide
MKLPVRLLVLMFWIPASSSDVLMTQIPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 239 - protein, 5 aa, Artificial Sequence
  Description of Artificial Sequence: Synthetic peptide
TYGMS

SEQ ID NO: 240 - protein, 17 aa, Artificial Sequence
  Description of Artificial Sequence: Synthetic peptide
WINTYSGVPTYADDFKG

SEQ ID NO: 241 - protein, 12 aa, Artificial Sequence
  Description of Artificial Sequence: Synthetic peptide
GRDGYQVAWFAY

SEQ ID NO: 242 - protein, 11 aa, Artificial Sequence
  Description of Artificial Sequence: Synthetic peptide
ITSTDIDDDMN

SEQ ID NO: 243 - protein, 7 aa, Artificial Sequence
  Description of Artificial Sequence: Synthetic peptide
EGNTLRP

SEQ ID NO: 244 - protein, 9 aa, Artificial Sequence
  Description of Artificial Sequence: Synthetic peptide
LQSDNLPYT

SEQ ID NO: 245 - protein, 121 aa, Artificial Sequence
  Description of Artificial Sequence: Synthetic polypeptide
QIQLVQSGPELKKPGEAVKISCKSSGYTFTTYGMSWVKQAPGRALKWMGWINTYSGVPTYADDFKGRFAFSLESSASTAYLQINNLKNEDTATYFCARGRDGYQVAWFAYWGQGTLVTVSA

SEQ ID NO: 246 - protein, 107 aa, Artificial Sequence
  Description of Artificial Sequence: Synthetic polypeptide
ETTVTQSPASLSMAIGDKVTIRCITSTDIDDDMNWFQQKPGEPPKLLISEGNTLRPGVPSRFSGSGYGTDFIFTIENMLSEDVADYYCLQSDNLPYTFGGGTKLEIK

SEQ ID NO: 247 - protein, 464 aa, Artificial Sequence
  Description of Artificial Sequence: Synthetic polypeptide
MGWLWNLLFLMAAAQSAQAQIQLVQSGPELKKPGEAVKISCKSSGYTFTTYGMSWVKQAPGRALKWMGWINTYSGVPTYADDFKGRFAFSLESSASTAYLQINNLKNEDTATYFCARGRDGYQVAWFAYWGQGTLVTVSAAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 248 - protein, 234 aa, Artificial Sequence
  Description of Artificial Sequence: Synthetic polypeptide
MFSLALLLSLLLLCVSDSRAETTVTQSPASLSMAIGDKVTIRCITSTDIDDDMNWFQQKPGEPPKLLISEGNTLRPGVPSRFSGSGYGTDFIFTIENMLSEDVADYYCLQSDNLPYTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 249 - protein, 5 aa, Artificial Sequence
  Description of Artificial Sequence: Synthetic peptide
NYWMH

SEQ ID NO: 250 - protein, 17 aa, Artificial Sequence
  Description of Artificial Sequence: Synthetic peptide
MIDPSDSYTNYNPKFKG

SEQ ID NO: 251 - protein, 115 aa, Artificial Sequence
  Description of Artificial Sequence: Synthetic polypeptide
QVQLQQPGAELVRPGTSVKLSCKASGYTFTNYWMHWVKQRPGQGLEWIGMIDPSDSYTNYNPKFKGKATLTVDTSSSTAYMQLSSLTSEDSAVYYCARNYSGDYWGQGTTLTVSS

SEQ ID NO: 252 - protein, 112 aa, Artificial Sequence
  Description of Artificial Sequence: Synthetic polypeptide
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIK

SEQ ID NO: 253 - protein, 458 aa, Artificial Sequence
  Description of Artificial Sequence: Synthetic polypeptide
MGWSCIIVLLVSTATGVHSQVQLQQPGAELVRPGTSVKLSCKASGYTFTNYWMHWVKQRPGQGLEWIGMIDPSDSYTNYNPKFKGKATLTVDTSSSTAYMQLSSLTSEDSAVYYCARNYSGDYWGQGTTLTVSSAKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 254 - protein, 238 aa, Artificial Sequence
  Description of Artificial Sequence: Synthetic polypeptide
MKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSYVPWTFGGGTKLEIKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC

SEQ ID NO: 255 - protein, 449 aa, Artificial Sequence
  Antibody heavy chain
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 256 - protein, 214 aa, Artificial Sequence
  Antibody light chain
DIQMTQSPSSLSASVGDRVTITCRASQEISGYLSWYQQKPGKAPKRLIYAASTLDSGVPSRFSGSGSGTEFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - protein, 119 aa, Artificial Sequence
  Description of Artificial Sequence: Synthetic polypeptide
EVQLVESGGGLVQPGGSLRLSCAASGFTFSDYAMSWVRQAPGKGLEWVATISDGGTYTYYPDNVKGRFTISRDNAKNSLYLQMNSLRAEDTAVYYCAREWGDYDGFDYWGQGTLVTVSS

SEQ ID NO: 258 - protein, 119 aa, Artificial Sequence
  Description of Artificial Sequence: Synthetic polypeptide
QVQLVESGGGLVKPGGSLRLSCAASGFTFSDYAMSWIRQAPGKGLEWVSTISDGGTYTYYPDNVKGRFTISRDNAKNSLYLQMNSLRAEDTAVYYCAREWGDYDGFDYWGQGTLVTVSS

SEQ ID NO: 259 - protein, 119 aa, Artificial Sequence; identical to SEQ ID NO: 199
  Description of Artificial Sequence: Synthetic polypeptide

SEQ ID NO: 260 - protein, 119 aa, Artificial Sequence
  Description of Artificial Sequence: Synthetic polypeptide
EVQLVESGGGLVKPGGSLRLSCAASGFTFSDYAMSWVRQAPGKGLEWVSTISDGGTYTYYPDNVKGRFTISRDNAKNSLYLQMNSLRAEDTAVYYCAREWGDYDGFDYWGQGTLVTVSS

SEQ ID NO: 261 - protein, 119 aa, Artificial Sequence
  Description of Artificial Sequence: Synthetic polypeptide
EVQLLESGGGLVQPGGSLRLSCAASGFTFSDYAMSWVRQAPGKGLEWVSTISDGGTYTYYPDNVKGRFTISRDNSKNTLYLQMNSLRAEDTAVYYCAREWGDYDGFDYWGQGTLVTVSS

SEQ ID NO: 262 - protein, 119 aa, Artificial Sequence
  Description of Artificial Sequence: Synthetic polypeptide
QVQLVESGGGVVQPGRSLRLSCAASGFTFSDYAMSWVRQAPGKGLEWVATISDGGTYTYYPDNVKGRFTISRDNSKNTLYLQMSSLRAEDTAVYYCAREWGDYDGFDYWGQGTLVTVSS

SEQ ID NO: 263 - protein, 119 aa, Artificial Sequence
  Description of Artificial Sequence: Synthetic polypeptide
EVQLVESGGGLVKPGGSLRLSCAASGFTFSDYAMSWVRQAPGKGLEWVATISDGGTYTYYPDNVKGRFTISRDNAKNSLYLQMNSLRAEDTAVYYCAREWGDYDGFDYWGQGTLVTVSS

SEQ ID NO: 264 - protein, 107 aa, Artificial Sequence
  Description of Artificial Sequence: Synthetic polypeptide
DIQLTQSPSFLSASVGDRVTITCRASQEISGYLSWYQQKPGKAPKLLIYAASTLDSGVPSRFSGSGSGTEFTLTISSLQPEDFATYYCLQYDSYPYTFGQGTKLEIK

SEQ ID NO: 265 - protein, 107 aa, Artificial Sequence
  Description of Artificial Sequence: Synthetic polypeptide
DIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIK

SEQ ID NO: 266 - protein, 107 aa, Artificial Sequence; identical to SEQ ID NO: 200
  Description of Artificial Sequence: Synthetic polypeptide

SEQ ID NO: 267 - protein, 107 aa, Artificial Sequence
  Description of Artificial Sequence: Synthetic polypeptide
DIQMTQSPSSLSASVGDRVTITCRASQEISGYLSWYQQKPGKAPKLLIYAASTLDSGVPSRFSGSGSGTDFTFTISSLQPEDIATYYCLQYDSYPYTFGQGTKLEIK

SEQ ID NO: 268 - protein, 107 aa, Artificial Sequence
  Description of Artificial Sequence: Synthetic polypeptide
DIQMTQSPSSLSASVGDRVTITCRASQEISGYLSWYQQKPGKAPKLLIYAASTLDSGVPSRFSGSGSGTDFTLTISSLQPEDFATYYCLQYDSYPYTFGQGTKLEIK

SEQ ID NO: 269 - protein, 107 aa, Artificial Sequence
  Description of Artificial Sequence: Synthetic polypeptide
DIQMTQSPSSLSASVGDRVTITCRASQEISGYLSWLQQKPGGAIKRLIYAASTLDSGVPSRFSGSGSGSDYTLTISSLQPEDFATYYCLQYDSYPYTFGQGTKLEIK

SEQ ID NO: 270 - protein, 468 aa, Artificial Sequence
  Description of Artificial Sequence: Synthetic polypeptide
MNFGLSLMFLVLVLKGVQCEVQLVESGGGLVKPGGSLKLSCAASGFTFSDYAMSWVRQTPEKRLEWVATISDGGTYTYYPDNVKGRFTISRDNAKNNLYLQMSHLKSEDTAMYYCAREWGDYDGFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 271 - protein, 236 aa, Artificial Sequence
  Description of Artificial Sequence: Synthetic polypeptide
MDMRVPAHVFGFLLLWFPGTRCDIQMTQSPSSLSASLGERVSLTCRASQEISGYLSWLQQKPDGTIKRLIYAASTLDSGVPKRFSGSRSGSDYSLTIGSLESEDLADYYCLQYDSYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 272 - protein, 471 aa, Artificial Sequence; identical to SEQ ID NO: 201
  Description of Artificial Sequence: Synthetic polypeptide

SEQ ID NO: 273 - protein, 467 aa, Artificial Sequence
  Description of Artificial Sequence: Synthetic polypeptide
MDMRVPAQLLGLLLLWLRGARCQVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 274 - protein, 236 aa, Artificial Sequence
  Description of Artificial Sequence: Synthetic polypeptide
MDMRVPAQLLGLLLLWLRGARCDIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 275 - protein, 236 aa, Artificial Sequence; identical to SEQ ID NO: 202
  Description of Artificial Sequence: Synthetic polypeptide

SEQ ID NO: 276 - protein, 449 aa, Artificial Sequence
  Antibody heavy chain no leader sequence
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 277 - protein, 445 aa, Artificial Sequence
  Antibody heavy chain no leader sequence
QVQLVESGGGLVKPGGSLRLSCAASGFTFSDYAMSWIRQAPGKGLEWVSTISDGGTYTYYPDSVKGRFTISRDNAKNSLYLQMNSLRAEDTAVYYCAREWGDYDGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 278 - protein, 214 aa, Artificial Sequence
  Antibody light chain no leader sequence
DIQMTQSPSSLSASVGDRVTITCRASQEISGYLSWFQQKPGKAPKSLIYAASTLDSGVPSRFSGSGSGTDFTLTISSLQPEDFATYYCLQYDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 279 - protein, 214 aa, Artificial Sequence; identical to SEQ ID NO: 256
  Antibody light chain no leader sequence

SEQ ID NO: 280 - protein, 450 aa, Artificial Sequence
  Antibody heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 281 - protein, 214 aa, Artificial Sequence
  Antibody light chain
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 448 aa, Artificial Sequence
  Antibody heavy chain
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 283 - protein, 214 aa, Artificial Sequence
  Antibody light chain
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 284 - protein, 447 aa, Artificial Sequence
  Antibody heavy chain
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 285 - protein, 220 aa, Artificial Sequence
  Antibody light chain
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 286 - protein, 445 aa, Artificial Sequence
  Antibody heavy chain
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 287 - protein, 217 aa, Artificial Sequence
  Antibody light chain
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 288 - protein, 449 aa, Artificial Sequence
  Antibody heavy chain
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 289 - protein, 220 aa, Artificial Sequence
  Antibody light chain
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 290 - protein, 446 aa, Artificial Sequence
  Antibody heavy chain
EVQLLESGGGLVQPGGSLRLSCAASGFTFSSYAMSWVRQAPGKGLEWVSAINSQGKSTYYADSVKGRFTISRDNSKNTLYLQMNSLRAEDTAVYYCARWGDEGFDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 291 - protein, 214 aa, Artificial Sequence
  Antibody light chain
DIQMTQSPSSLSASVGDRVTITCRASQGISNWLAWYQQKPGKAPKLLIYGASSLQSGVPSRFSGSGSGTDFTLTISSLQPEDFATYYCQQYSSFPTTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 292 - DNA, 25 bp, Artificial Sequence
  Morpholino
cgtcacaggcaggacccactgccca

SEQ ID NO: 293 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 294 - protein, 345 aa, Artificial Sequence
  Fusion protein
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 295 - protein, 345 aa, Artificial Sequence; identical to SEQ ID NO: 294
  Fusion protein

SEQ ID NO: 296 - protein, 347 aa, Artificial Sequence
  Fusion protein
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 297 - protein, 119 aa, Artificial Sequence
  Heavy chain variable region
EVQLVESGGGVVQPGRSLRLSCSSSGFTFSDYYMYWVRQAPGKGLEWVAYMSNVGAITDYPDTVKGRFTISRDNSKNTLFLQMDSLRPEDTGVYFCARGTRDGSWFAYWGQGTPVTVSS

SEQ ID NO: 298 - protein, 119 aa, Artificial Sequence
  Heavy chain variable region
EVQLVESGGGVVQPGRSLRLSCSTSGFTFSDYYMYWVRQAPGKGLEWVAYMSNVGAITDYPDTVKGRFTISRDNSKNTLFLQMDSLRPEDTGVYFCARGTRDGSWFAYWGQGTPVTVSS

SEQ ID NO: 299 - protein, 119 aa, Artificial Sequence
  Heavy chain variable region
EVQLVESGGGVVQPGRSLRLSCSSSGFTFSDYYMYWVRQAPGKGLEWVAYMSNVGAITDYPDTVKGRFTISRDNAKNTLFLQMDSLRPEDTGVYFCARGTRDGSWFAYWGQGTPVTVSS

SEQ ID NO: 300 - protein, 119 aa, Artificial Sequence
  Heavy chain variable region
EVQLVESGGGVVQPGRSLRLSCSSSGFTFSDYYMYWVRQAPGKGLEWVAYMSNVGAITDYPDTVKGRFTISRDNAKSTLFLQMDSLRPEDTGVYFCARGTRDGSWFAYWGQGTPVTVSS

SEQ ID NO: 301 - protein, 119 aa, Artificial Sequence
  Heavy chain variable region
EVQLVESGGGVVQPGRSLRLSCSSSGFTFSDYYMYWVRQAPGKGLEWVAYMSNVGAITDYPDTVKGRFTISRDNAKSTLYLQMDSLRPEDTGVYFCARGTRDGSWFAYWGQGTPVTVSS

SEQ ID NO: 302 - protein, 112 aa, Artificial Sequence
  Light chain variable region
DIQMTQSPSSLSASVGDRVTITCRSSQRIVHSNGNTYLEWYQQTPGKAPKLLIYKVSNRFSGVPSRFSGSGSGTDYTFTISSLQPEDIATYYCFQGSHVPFTFGQGTKLQIT

SEQ ID NO: 303 - protein, 112 aa, Artificial Sequence
  Light chain variable region
DIQMTQSPSSLSASVGDRVTITCRSSQRIVHSNGNTYLEWYQQTPGKAPKLLIYKVSNRFSGVPSRFSGSGSGTDFTFTISSLQPEDIATYYCFQGSHVPFTFGQGTKLQIT

SEQ ID NO: 304 - protein, 10 aa, Artificial Sequence
  Heavy chain N-terminal region
EVQLVESGGG

SEQ ID NO: 305 - protein, 5 aa, Artificial Sequence
  Heavy chain CDR1
DYYMY

SEQ ID NO: 306 - protein, 17 aa, Artificial Sequence
  Heavy chain CDR2
YMSNVGAITDYPDTVKG

SEQ ID NO: 307 - protein, 10 aa, Artificial Sequence
  Heavy chain CDR3
GTRDGSWFAY

SEQ ID NO: 308 - protein, 10 aa, Artificial Sequence
  Light chain N-terminal region
DIQMTQSPSS

SEQ ID NO: 309 - protein, 16 aa, Artificial Sequence
  Light chain CDR1
RSSQRIVHSNGNTYLE

SEQ ID NO: 310 - protein, 7 aa, Artificial Sequence
  Light chain CDR2
KVSNRFS

SEQ ID NO: 311 - protein, 9 aa, Artificial Sequence
  Light chain CDR3
FQGSHVPFT

SEQ ID NO: 312 - protein, 672 aa, Artificial Sequence
  Mutated and Conjugated Protein
  MISC_FEATURE (459)..(672): Light Chain
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTTSIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 313 - protein, 458 aa, Artificial Sequence
  Synthetic polypeptide
EVQLQQSGPDLVKPGASVKISCKASGYSFTGYYMHWVKQSPGKGLEWIGRINPNNGVTLYNQKFKDKATLTVDKSSTTAYMELRSLTSEDSAVYYCARSTMITNYVMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDSGGPSEKSEEINEKDLRKKSELQGTALGNLKQIYYYNSKAITSSEKSADQFLTNTLLFKGFFTGHPWYNDLLVDLGSTAATSEYEGSSVDLYGAYYGYQCAGGTPNKTACMYGGVTLHDNNRLTEEKKVPINLWIDGKQTTVPIDKVKTSKKEVTVQELDLQARHYLHGKFGLYNSDSFGGKVQRGLIVFHSSEGSTVSYDLFDAQGQYPDTLLRIYRDNTTISSTSLSISLYLYTT

SEQ ID NO: 314 - protein, 214 aa, Artificial Sequence
  Synthetic polypeptide
SIVMTQTPTSLLVSAGDRVTITCKASQSVSNDVAWYQQKPGQSPKLLISYTSSRYAGVPDRFSGSGYGTDFTLTISSVQAEDAAVYFCQQDYNSPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNES

SEQ ID NO: 315 - protein, 7 aa, Artificial Sequence
  MUC1 epitope
PDTRPAP

SEQ ID NO: 316 - protein, 9 aa, Artificial Sequence
  MUC1 epitope
STAPPVHNV

SEQ ID NO: 317 - protein, 5 aa, Artificial Sequence
  MUC1 epitope
PDTRP

SEQ ID NO: 318 - protein, 6 aa, Artificial Sequence
  MUC1 epitope
DTRPAP

SEQ ID NO: 319 - protein, 7 aa, Artificial Sequence
  MUC1 epitope
DTNRPAP